SEQUENCE LISTING (523 sequences)

SEQ ID NO: 1 - DNA, 7889 bp, synthetic construct
  misc_feature 1..7889: note = Cypor human factor 9 gene ride
  source 1..7889: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtaaagatctgatatcatcgatcgcgatgcattaattaagcggccgctgtacataggaggttcgaaccctgctgaagggagaggttccaatactacaaaatgtagcgggatattgtcatcacctttggggacatgtcatcatggtccccagacagagttacaaaactcatcccctacacagcactatgtctctggtactgtttgttctacagatgtcaacaacagaggcccagccatctcctattgcttggcttgtcagtctttctagcctccccattattaatttcaaatggggcaggtgttaggagggcaaaaatccacatattaagtgcaaagcctttcaggagatttcctgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggaagcggcgccaccaatttcagcctgctgaaacaggccggcgacgtggaagagaaccctggccctgctagccagcgcgtgaacatgattatggccgagagccctggcctgatcaccatctgcctgctgggctacctgctgagcgccgagtgtaccgtgttcctggaccacgagaacgccaacaagatcctgaacagacccaagagatacaacagcggcaagctggaagagttcgtgcagggcaacctggaacgcgagtgcatggaagagaagtgcagcttcgaagaggccagagaggtgttcgagaacaccgagagaaccaccgagttctggaagcagtacgtggacggcgaccagtgcgagagcaacccttgtctgaacggcggcagctgcaaggacgacatcaacagctacgagtgctggtgccccttcggcttcgagggcaagaactgcgagctggacgtgacctgcaacatcaagaacggcagatgcgagcagttctgcaagaacagcgccgacaacaaggtcgtgtgctcctgcaccgagggctacagactggccgagaaccagaagtcctgcgagcccgctgtgcctttcccatgcggaagagtgtccgtgtcccagaccagcaagctgaccagagccgagacagtgttccccgacgtggactacgtgaacagcaccgaggccgagacaatcctggacaacatcacccagagcacccagtccttcaacgacttcaccagagtcgtgggcggcgaggatgctaagcctggccagttcccgtggcaggtggtgctgaacggaaaggtggacgccttctgcggcggctccatcgtgaacgagaagtggatcgtgacagccgcccactgcgtggaaaccggcgtgaagatcacagtggtggccggcgagcacaacatcgaggaaaccgagcacacagagcagaaaagaaacgtgatcaggatcatcccccaccacaactacaacgccgccatcaacaagtacaaccacgatatcgccctgctggaactggacgagcccctggtgctgaatagctacgtgacccccatctgtatcgccgacaaagagtacaccaacatctttctgaagttcggcagcggctacgtgtccggctggggcagagtgtttcacaagggcagatccgctctggtgctgcagtacctgagagtgcctctggtggacagagccacctgtctgagaagcaccaagttcaccatctacaacaacatgttctgcgctggcttccacgagggcggcagagactcttgtcagggcgattctggcggccctcacgtgacagaggtggaaggcaccagctttctgaccggcatcatcagctggggcgaggaatgcgccatgaaggggaagtacggcatctacaccaaggtgtccagatacgtgaactggatcaaagaaaagaccaagctgacataagctagcttagcctaaacacatcacaaccacaaccttctcaggtaactaaccggtccacgactgatgagtccgtgaggacgaaacgagtaagctcgtctcgtgggggtcctgacctacgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggaccttaagccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatagacataaggctatttcaaaattatttcagttttagaaagaattgaaagttttgtagcattctgagaagacagctttcatttgtaatcataggtaatatgtaggtcctcagaaatggtgagacccctgactttgacacttggggactctgagggaccagtgatgaagagggcacaacttatatcacacatgcacgagttggggtgagagggtgtcacaacatctatcagtgtgtcatctgcccaccaagtaacagatgtcagctaagactaggtcatgtgtaggctgtctacaccagtgaaaatcgcaaaaagaatctaagaaattccacatttctagaaaataggtttggaaaccgtattccattttacaaaggacacttacatttctctttttgttttccaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctactctgtggttcagaactctatcttccaaaggcgcgcttcaccctagcagcctctttggctcagaggaatccctgcctttcctcccttcatctcagcagagaatgtagttccacatgggactagtgtacacgcgtgatatcagatctgttacgtagataagtagcatggcgggttaatcattaactacaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaagcgcgcagctgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgcttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggcagctgcgcgctcgaacttcatgcctgccgaccttccccaggtcacgatccggacggcgggtgagttcacattttarcagccggacgtgcaractccgctggtggtctaacgtcggttaggtcccttgaatcacgggacatatgttggtgttggaggt

SEQ ID NO: 2 - DNA, 19 bp, synthetic construct
  misc_feature 1..19: note = Cypor shRNA target sequence
  source 1..19: mol_type = other DNA
ggtagcgcctcagtggtgt

SEQ ID NO: 3 - DNA, 19 bp, synthetic construct
  misc_feature 1..19: note = Cypor shRNA target sequence
  source 1..19: mol_type = other DNA
ggcaaggagctgtacctga

SEQ ID NO: 4 - DNA, 19 bp, synthetic construct
  misc_feature 1..19: note = Cypor shRNA target sequence
  source 1..19: mol_type = other DNA
gcaaggagctgtacctgaa

SEQ ID NO: 5 - DNA, 19 bp, synthetic construct
  misc_feature 1..19: note = Cypor shRNA target sequence
  source 1..19: mol_type = other DNA
aaggagctgtacctgagca

SEQ ID NO: 6 - DNA, 19 bp, synthetic construct
  misc_feature 1..19: note = Cypor shRNA target sequence
  source 1..19: mol_type = other DNA
aggagctgtacctgagcta

SEQ ID NO: 7 - DNA, 19 bp, synthetic construct
  misc_feature 1..19: note = Cypor shRNA target sequence
  source 1..19: mol_type = other DNA
ggaacatcatcgtgttcta

SEQ ID NO: 8 - DNA, 19 bp, synthetic construct
  misc_feature 1..19: note = Cypor shRNA target sequence
  source 1..19: mol_type = other DNA
ctccaaaatcaggtatgaa

SEQ ID NO: 9 - DNA, 19 bp, synthetic construct
  misc_feature 1..19: note = Cypor shRNA target sequence
  source 1..19: mol_type = other DNA
gcacccttcataggcttca

SEQ ID NO: 10 - DNA, 19 bp, synthetic construct
  misc_feature 1..19: note = Cypor shRNA target sequence
  source 1..19: mol_type = other DNA
gtgatttccagtgagtgta

SEQ ID NO: 11 - DNA, 19 bp, synthetic construct
  misc_feature 1..19: note = Cypor shRNA target sequence
  source 1..19: mol_type = other DNA
gtggttttctgcatggcca

SEQ ID NO: 12 - DNA, 23 bp, synthetic construct
  misc_feature 1..23: note = Cypor shRNA target sequence
  source 1..23: mol_type = other DNA
gaatgggtgcttcttgttggact

SEQ ID NO: 13 - DNA, 24 bp, synthetic construct
  misc_feature 1..24: note = Cypor shRNA target sequence
  source 1..24: mol_type = other DNA
ggccggctgaagagctacgagaac

SEQ ID NO: 14 - DNA, 22 bp, synthetic construct
  misc_feature 1..22: note = Cypor shRNA target sequence
  source 1..22: mol_type = other DNA
gcacctgtggaagttgatcgaa

SEQ ID NO: 15 - DNA, 23 bp, synthetic construct
  misc_feature 1..23: note = Cypor shRNA target sequence
  source 1..23: mol_type = other DNA
cctaacctactggttcctcttct

SEQ ID NO: 16 - DNA, 24 bp, synthetic construct
  misc_feature 1..24: note = Cypor shRNA target sequence
  source 1..24: mol_type = other DNA
cggtggccgaagaagtatctcttt

SEQ ID NO: 17 - DNA, 21 bp, synthetic construct
  misc_feature 1..21: note = Cypor shRNA target sequence
  source 1..21: mol_type = other DNA
gccagcgtttcatgatcaaca

SEQ ID NO: 18 - DNA, 21 bp, synthetic construct
  misc_feature 1..21: note = Cypor shRNA target sequence
  source 1..21: mol_type = other DNA
gaacaagacctacgagcactt

SEQ ID NO: 19 - DNA, 21 bp, synthetic construct
  misc_feature 1..21: note = Cypor shRNA target sequence
  source 1..21: mol_type = other DNA
ggaacttggaggaggacttca

SEQ ID NO: 20 - DNA, 21 bp, synthetic construct
  misc_feature 1..21: note = Cypor shRNA target sequence
  source 1..21: mol_type = other DNA
ggatgaggagtccaacaagaa

SEQ ID NO: 21 - DNA, 21 bp, synthetic construct
  misc_feature 1..21: note = Cypor shRNA target sequence
  source 1..21: mol_type = other DNA
ggcggtggactacatcaagaa

SEQ ID NO: 22 - DNA, 21 bp, synthetic construct
  misc_feature 1..21: note = Cypor shRNA target sequence
  source 1..21: mol_type = other DNA
gcggtggactacatcaagaaa

SEQ ID NO: 23 - DNA, 21 bp, synthetic construct
  misc_feature 1..21: note = Cypor shRNA target sequence
  source 1..21: mol_type = other DNA
ggtggactacatcaagaaact

SEQ ID NO: 24 - DNA, 21 bp, synthetic construct
  misc_feature 1..21: note = Cypor shRNA target sequence
  source 1..21: mol_type = other DNA
gcccttggaataaagttctgt

SEQ ID NO: 25 - DNA, 20 bp, synthetic construct
  misc_feature 1..20: note = Cypor shRNA target sequence
  source 1..20: mol_type = other DNA
gccgaagaagtatctctttc

SEQ ID NO: 26 - DNA, 19 bp, synthetic construct
  misc_feature 1..19: note = Cypor shRNA target sequence
  source 1..19: mol_type = other DNA
ggttcctcttcagaaagaa

SEQ ID NO: 27 - DNA, 19 bp, synthetic construct
  misc_feature 1..19: note = Cypor shRNA target sequence
  source 1..19: mol_type = other DNA
ggtggactacatcaagaaa

SEQ ID NO: 28 - DNA, 19 bp, synthetic construct
  misc_feature 1..19: note = Cypor shRNA target sequence
  source 1..19: mol_type = other DNA
gctgccagcgtttcatgat

SEQ ID NO: 29 - DNA, 19 bp, synthetic construct
  misc_feature 1..19: note = Cypor shRNA target sequence
  source 1..19: mol_type = other DNA
gccagcgtttcatgatcaa

SEQ ID NO: 30 - DNA, 19 bp, synthetic construct
  misc_feature 1..19: note = Cypor shRNA target sequence
  source 1..19: mol_type = other DNA
ggccgaagaagtatctctt

SEQ ID NO: 31 - DNA, 20 bp, synthetic construct
  misc_feature 1..20: note = Cypor shRNA target sequence
  source 1..20: mol_type = other DNA
ggccgtgtgtgaacactttg

SEQ ID NO: 32 - DNA, 19 bp, synthetic construct
  misc_feature 1..19: note = Cypor shRNA target sequence
  source 1..19: mol_type = other DNA
ggaggaacatcatcgtgtt

SEQ ID NO: 33 - DNA, 20 bp, synthetic construct
  misc_feature 1..20: note = Cypor shRNA target sequence
  source 1..20: mol_type = other DNA
ggtcaagttcgcggtgtttg

SEQ ID NO: 34 - DNA, 23 bp, synthetic construct
  misc_feature 1..23: note = Cypor CRISPR target sequences
  source 1..23: mol_type = other DNA
tacagcacgttggtacgcggcgg

SEQ ID NO: 35 - DNA, 23 bp, synthetic construct
  misc_feature 1..23: note = Cypor CRISPR target sequences
  source 1..23: mol_type = other DNA
tggccgcatcaacaagggcgtgg

SEQ ID NO: 36 - DNA, 23 bp, synthetic construct
  misc_feature 1..23: note = Cypor CRISPR target sequences
  source 1..23: mol_type = other DNA
tcataggcttcatccaggagcgg

SEQ ID NO: 37 - DNA, 23 bp, synthetic construct
  misc_feature 1..23: note = Cypor CRISPR target sequences
  source 1..23: mol_type = other DNA
ctgtggaagttgatcgaaggcgg

SEQ ID NO: 38 - DNA, 23 bp, synthetic construct
  misc_feature 1..23: note = Cypor CRISPR target sequences
  source 1..23: mol_type = other DNA
tgcggccaaggtgtacatggggg

SEQ ID NO: 39 - DNA, 23 bp, synthetic construct
  misc_feature 1..23: note = Cypor CRISPR target sequences
  source 1..23: mol_type = other DNA
acaggtagtcctcatccgagcgg

SEQ ID NO: 40 - DNA, 23 bp, synthetic construct
  misc_feature 1..23: note = Cypor CRISPR target sequences
  source 1..23: mol_type = other DNA
tgggcaagtacgtggacaagcgg

SEQ ID NO: 41 - DNA, 23 bp, synthetic construct
  misc_feature 1..23: note = Cypor CRISPR target sequences
  source 1..23: mol_type = other DNA
tagtagcgggcctgcaggcgcgg

SEQ ID NO: 42 - DNA, 23 bp, synthetic construct
  misc_feature 1..23: note = Cypor CRISPR target sequences
  source 1..23: mol_type = other DNA
gtcccacaaggtgagacgggcgg

SEQ ID NO: 43 - DNA, 23 bp, synthetic construct
  misc_feature 1..23: note = Cypor CRISPR target sequences
  source 1..23: mol_type = other DNA
acggattcttggcatcaaagggg

SEQ ID NO: 44 - DNA, 23 bp, synthetic construct
  misc_feature 1..23: note = Cypor CRISPR target sequences
  source 1..23: mol_type = other DNA
ggtccacaccgacatagatgcgg

SEQ ID NO: 45 - DNA, 23 bp, synthetic construct
  misc_feature 1..23: note = Cypor CRISPR target sequences
  source 1..23: mol_type = other DNA
tgaggcgatggagtagtagcggg

SEQ ID NO: 46 - DNA, 23 bp, synthetic construct
  misc_feature 1..23: note = Cypor CRISPR target sequences
  source 1..23: mol_type = other DNA
tgttctacggctcccagacgggg

SEQ ID NO: 47 - DNA, 23 bp, synthetic construct
  misc_feature 1..23: note = Cypor CRISPR target sequences
  source 1..23: mol_type = other DNA
gatgcggccaaggtgtacatggg

SEQ ID NO: 48 - DNA, 23 bp, synthetic construct
  misc_feature 1..23: note = Cypor CRISPR target sequences
  source 1..23: mol_type = other DNA
cctgtcatcatggtgggccccgg

SEQ ID NO: 49 - DNA, 23 bp, synthetic construct
  misc_feature 1..23: note = Cypor CRISPR target sequences
  source 1..23: mol_type = other DNA
tgcctcgcatcccgtagcggtgg

SEQ ID NO: 50 - DNA, 23 bp, synthetic construct
  misc_feature 1..23: note = Cypor CRISPR target sequences
  source 1..23: mol_type = other DNA
gcctcgcatcccgtagcggtggg

SEQ ID NO: 51 - DNA, 23 bp, synthetic construct
  misc_feature 1..23: note = Cypor CRISPR target sequences
  source 1..23: mol_type = other DNA
cgaacatgggcaccagcgcacgg

SEQ ID NO: 52 - DNA, 23 bp, synthetic construct
  misc_feature 1..23: note = Cypor CRISPR target sequences
  source 1..23: mol_type = other DNA
ccctggacgtgtggagctagggg

SEQ ID NO: 53 - DNA, 23 bp, synthetic construct
  misc_feature 1..23: note = Cypor CRISPR target sequences
  source 1..23: mol_type = other DNA
ggttctcgtagctcttcagccgg

SEQ ID NO: 54 - DNA, 23 bp, synthetic construct
  misc_feature 1..23: note = Cypor CRISPR target sequences
  source 1..23: mol_type = other DNA
agggccgtgcggtaggacgtagg

SEQ ID NO: 55 - DNA, 23 bp, synthetic construct
  misc_feature 1..23: note = Cypor CRISPR target sequences
  source 1..23: mol_type = other DNA
cgtgttctacggctcccagacgg

SEQ ID NO: 56 - DNA, 23 bp, synthetic construct
  misc_feature 1..23: note = Cypor CRISPR target sequences
  source 1..23: mol_type = other DNA
tcgtacagcacgttggtacgcgg

SEQ ID NO: 57 - DNA, 23 bp, synthetic construct
  misc_feature 1..23: note = Cypor CRISPR target sequences
  source 1..23: mol_type = other DNA
gtgcggtaggacgtagggcacgg

SEQ ID NO: 58 - DNA, 23 bp, synthetic construct
  misc_feature 1..23: note = Cypor CRISPR target sequences
  source 1..23: mol_type = other DNA
ctacgagcacttcaatgccatgg

SEQ ID NO: 59 - DNA, 23 bp, synthetic construct
  misc_feature 1..23: note = Cypor CRISPR target sequences
  source 1..23: mol_type = other DNA
tgcggtaggacgtagggcacggg

SEQ ID NO: 60 - DNA, 23 bp, synthetic construct
  misc_feature 1..23: note = Cypor CRISPR target sequences
  source 1..23: mol_type = other DNA
acatgcctcgcatcccgtagcgg

SEQ ID NO: 61 - DNA, 23 bp, synthetic construct
  misc_feature 1..23: note = Cypor CRISPR target sequences
  source 1..23: mol_type = other DNA
agcagtcccacaaggtgagacgg

SEQ ID NO: 62 - DNA, 23 bp, synthetic construct
  misc_feature 1..23: note = Cypor CRISPR target sequences
  source 1..23: mol_type = other DNA
atgaggcgatggagtagtagcgg

SEQ ID NO: 63 - DNA, 23 bp, synthetic construct
  misc_feature 1..23: note = Cypor CRISPR target sequences
  source 1..23: mol_type = other DNA
caagtacgtggacaagcggctgg

SEQ ID NO: 64 - DNA, 23 bp, synthetic construct
  misc_feature 1..23: note = Cypor CRISPR target sequences
  source 1..23: mol_type = other DNA
atgcggccaaggtgtacatgggg

SEQ ID NO: 65 - DNA, 23 bp, synthetic construct
  misc_feature 1..23: note = Cypor CRISPR target sequences
  source 1..23: mol_type = other DNA
atcatggtgggccccggcaccgg

SEQ ID NO: 66 - DNA, 23 bp, synthetic construct
  misc_feature 1..23: note = Cypor CRISPR target sequences
  source 1..23: mol_type = other DNA
gccagagatcgacaacgccctgg

SEQ ID NO: 67 - DNA, 23 bp, synthetic construct
  misc_feature 1..23: note = Cypor CRISPR target sequences
  source 1..23: mol_type = other DNA
tgggacttgcgcacgaacatggg

SEQ ID NO: 68 - DNA, 23 bp, synthetic construct
  misc_feature 1..23: note = Cypor CRISPR target sequences
  source 1..23: mol_type = other DNA
tccacacgtccagggagtagcgg

SEQ ID NO: 69 - DNA, 23 bp, synthetic construct
  misc_feature 1..23: note = Cypor CRISPR target sequences
  source 1..23: mol_type = other DNA
caccgacatagatgcggccaagg

SEQ ID NO: 70 - DNA, 23 bp, synthetic construct
  misc_feature 1..23: note = Cypor CRISPR target sequences
  source 1..23: mol_type = other DNA
ggggagacgctgctgtactacgg

SEQ ID NO: 71 - DNA, 23 bp, synthetic construct
  misc_feature 1..23: note = Cypor CRISPR target sequences
  source 1..23: mol_type = other DNA
agagatcgacaacgccctggtgg

SEQ ID NO: 72 - DNA, 23 bp, synthetic construct
  misc_feature 1..23: note = Cypor CRISPR target sequences
  source 1..23: mol_type = other DNA
ggtacagctcctaagagacacgg

SEQ ID NO: 73 - DNA, 23 bp, synthetic construct
  misc_feature 1..23: note = Cypor CRISPR target sequences
  source 1..23: mol_type = other DNA
acagcacgttggtacgcggcggg

SEQ ID NO: 74 - DNA, 23 bp, synthetic construct
  misc_feature 1..23: note = Cypor CRISPR target sequences
  source 1..23: mol_type = other DNA
cgtactggcgaatgctggaaagg

SEQ ID NO: 75 - DNA, 23 bp, synthetic construct
  misc_feature 1..23: note = Cypor CRISPR target sequences
  source 1..23: mol_type = other DNA
gtgccctacgtcctaccgcacgg

SEQ ID NO: 76 - DNA, 23 bp, synthetic construct
  misc_feature 1..23: note = Cypor CRISPR target sequences
  source 1..23: mol_type = other DNA
tccaaggacgcccaccgctacgg

SEQ ID NO: 77 - DNA, 23 bp, synthetic construct
  misc_feature 1..23: note = Cypor CRISPR target sequences
  source 1..23: mol_type = other DNA
tcccacaaggtgagacgggcggg

SEQ ID NO: 78 - DNA, 23 bp, synthetic construct
  misc_feature 1..23: note = Cypor CRISPR target sequences
  source 1..23: mol_type = other DNA
taggacgtagggcacgggaatgg

SEQ ID NO: 79 - DNA, 23 bp, synthetic construct
  misc_feature 1..23: note = Cypor CRISPR target sequences
  source 1..23: mol_type = other DNA
caccccaaagtgttcacacacgg

SEQ ID NO: 80 - DNA, 23 bp, synthetic construct
  misc_feature 1..23: note = Cypor CRISPR target sequences
  source 1..23: mol_type = other DNA
gcagtcccacaaggtgagacggg

SEQ ID NO: 81 - DNA, 23 bp, synthetic construct
  misc_feature 1..23: note = Cypor CRISPR target sequences
  source 1..23: mol_type = other DNA
ggacttctacgactggctgcagg

SEQ ID NO: 82 - DNA, 23 bp, synthetic construct
  misc_feature 1..23: note = Cypor CRISPR target sequences
  source 1..23: mol_type = other DNA
tcgtgggtctcctaacctactgg

SEQ ID NO: 83 - DNA, 23 bp, synthetic construct
  misc_feature 1..23: note = Cypor CRISPR target sequences
  source 1..23: mol_type = other DNA
ctctgctctcgtcaaccagctgg

SEQ ID NO: 84 - DNA, 23 bp, synthetic construct
  misc_feature 1..23: note = Cypor CRISPR target sequences
  source 1..23: mol_type = other DNA
tctctctggggtcaagttcgcgg

SEQ ID NO: 85 - DNA, 23 bp, synthetic construct
  misc_feature 1..23: note = Cypor CRISPR target sequences
  source 1..23: mol_type = other DNA
cggatgaggactacctgtaccgg

SEQ ID NO: 86 - DNA, 23 bp, synthetic construct
  misc_feature 1..23: note = Cypor CRISPR target sequences
  source 1..23: mol_type = other DNA
cgccttcgatcaacttccacagg

SEQ ID NO: 87 - DNA, 23 bp, synthetic construct
  misc_feature 1..23: note = Cypor CRISPR target sequences
  source 1..23: mol_type = other DNA
gtaggtgagggccgtgcggtagg

SEQ ID NO: 88 - DNA, 23 bp, synthetic construct
  misc_feature 1..23: note = Cypor CRISPR target sequences
  source 1..23: mol_type = other DNA
ctgggacttgcgcacgaacatgg

SEQ ID NO: 89 - DNA, 23 bp, synthetic construct
  misc_feature 1..23: note = Cypor CRISPR target sequences
  source 1..23: mol_type = other DNA
atgtccctgaacaacctggatgg

SEQ ID NO: 90 - DNA, 23 bp, synthetic construct
  misc_feature 1..23: note = Cypor CRISPR target sequences
  source 1..23: mol_type = other DNA
ccaaggacgcccaccgctacggg

SEQ ID NO: 91 - DNA, 23 bp, synthetic construct
  misc_feature 1..23: note = Cypor CRISPR target sequences
  source 1..23: mol_type = other DNA
caccttggccgcatctatgtcgg

SEQ ID NO: 92 - DNA, 23 bp, synthetic construct
  misc_feature 1..23: note = Cypor CRISPR target sequences
  source 1..23: mol_type = other DNA
gcctatgaagggtgccaccccgg

SEQ ID NO: 93 - DNA, 23 bp, synthetic construct
  misc_feature 1..23: note = Cypor CRISPR target sequences
  source 1..23: mol_type = other DNA
tccctccaggtatgaatctgggg

SEQ ID NO: 94 - DNA, 23 bp, synthetic construct
  misc_feature 1..23: note = Cypor CRISPR target sequences
  source 1..23: mol_type = other DNA
tggccgcatctatgtcggtgtgg

SEQ ID NO: 95 - DNA, 23 bp, synthetic construct
  misc_feature 1..23: note = Cypor CRISPR target sequences
  source 1..23: mol_type = other DNA
gcagtacgcctcggagccctcgg

SEQ ID NO: 96 - DNA, 23 bp, synthetic construct
  misc_feature 1..23: note = Cypor CRISPR target sequences
  source 1..23: mol_type = other DNA
cattcgccagtacgagcttgtgg

SEQ ID NO: 97 - DNA, 23 bp, synthetic construct
  misc_feature 1..23: note = Cypor CRISPR target sequences
  source 1..23: mol_type = other DNA
aggaacatcatcgtgttctacgg

SEQ ID NO: 98 - DNA, 23 bp, synthetic construct
  misc_feature 1..23: note = Cypor CRISPR target sequences
  source 1..23: mol_type = other DNA
cacgttggtacgcggcgggttgg

SEQ ID NO: 99 - DNA, 23 bp, synthetic construct
  misc_feature 1..23: note = Cypor CRISPR target sequences
  source 1..23: mol_type = other DNA
ctcagccacgatgtcgtagaagg

SEQ ID NO: 100 - DNA, 23 bp, synthetic construct
  misc_feature 1..23: note = Cypor CRISPR target sequences
  source 1..23: mol_type = other DNA
gtctgaattttggtgaactcggg

SEQ ID NO: 101 - DNA, 23 bp, synthetic construct
  misc_feature 1..23: note = Cypor CRISPR target sequences
  source 1..23: mol_type = other DNA
gtccagggagtagcggcccttgg

SEQ ID NO: 102 - DNA, 23 bp, synthetic construct
  misc_feature 1..23: note = Cypor CRISPR target sequences
  source 1..23: mol_type = other DNA
gtgttctacggctcccagacggg

SEQ ID NO: 103 - DNA, 23 bp, synthetic construct
  misc_feature 1..23: note = Cypor CRISPR target sequences
  source 1..23: mol_type = other DNA
cggttccctggttcagcttccgg

SEQ ID NO: 104 - DNA, 23 bp, synthetic construct
  misc_feature 1..23: note = Cypor CRISPR target sequences
  source 1..23: mol_type = other DNA
gcacctggaattggacatctcgg

SEQ ID NO: 105 - DNA, 23 bp, synthetic construct
  misc_feature 1..23: note = Cypor CRISPR target sequences
  source 1..23: mol_type = other DNA
tacgacatcgtggctgagctcgg

SEQ ID NO: 106 - DNA, 23 bp, synthetic construct
  misc_feature 1..23: note = Cypor CRISPR target sequences
  source 1..23: mol_type = other DNA
tcttcctgcccagggatgcacgg

SEQ ID NO: 107 - DNA, 23 bp, synthetic construct
  misc_feature 1..23: note = Cypor CRISPR target sequences
  source 1..23: mol_type = other DNA
caactctgtgcacatctgtgcgg

SEQ ID NO: 108 - DNA, 23 bp, synthetic construct
  misc_feature 1..23: note = Cypor CRISPR target sequences
  source 1..23: mol_type = other DNA
gcccgcccgtctcaccttgtggg

SEQ ID NO: 109 - DNA, 23 bp, synthetic construct
  misc_feature 1..23: note = Cypor CRISPR target sequences
  source 1..23: mol_type = other DNA
gtagaagtcctgggcattgtcgg

SEQ ID NO: 110 - DNA, 23 bp, synthetic construct
  misc_feature 1..23: note = Cypor CRISPR target sequences
  source 1..23: mol_type = other DNA
gtactacggctgccgccgctcgg

SEQ ID NO: 111 - DNA, 23 bp, synthetic construct
  misc_feature 1..23: note = Cypor CRISPR target sequences
  source 1..23: mol_type = other DNA
gcacgcggtggcagcttaccagg

SEQ ID NO: 112 - DNA, 23 bp, synthetic construct
  misc_feature 1..23: note = Cypor CRISPR target sequences
  source 1..23: mol_type = other DNA
ggtagtcctcatccgagcggcgg

SEQ ID NO: 113 - DNA, 23 bp, synthetic construct
  misc_feature 1..23: note = Cypor CRISPR target sequences
  source 1..23: mol_type = other DNA
cataggcttcatccaggagcggg

SEQ ID NO: 114 - DNA, 23 bp, synthetic construct
  misc_feature 1..23: note = Cypor CRISPR target sequences
  source 1..23: mol_type = other DNA
aaggctggccgcatcaacaaggg

SEQ ID NO: 115 - DNA, 23 bp, synthetic construct
  misc_feature 1..23: note = Cypor CRISPR target sequences
  source 1..23: mol_type = other DNA
atggcctcctcctccggcgaggg

SEQ ID NO: 116 - DNA, 23 bp, synthetic construct
  misc_feature 1..23: note = Cypor CRISPR target sequences
  source 1..23: mol_type = other DNA
aaacacctgcgtggacacagggg

SEQ ID NO: 117 - DNA, 23 bp, synthetic construct
  misc_feature 1..23: note = Cypor CRISPR target sequences
  source 1..23: mol_type = other DNA
cgagctggcgcagtacgcctcgg

SEQ ID NO: 118 - DNA, 23 bp, synthetic construct
  misc_feature 1..23: note = Cypor CRISPR target sequences
  source 1..23: mol_type = other DNA
gcgtggccaccaactggctgcgg

SEQ ID NO: 119 - DNA, 23 bp, synthetic construct
  misc_feature 1..23: note = Cypor CRISPR target sequences
  source 1..23: mol_type = other DNA
cgggatgcgaggcatgtcagcgg

SEQ ID NO: 120 - DNA, 23 bp, synthetic construct
  misc_feature 1..23: note = Cypor CRISPR target sequences
  source 1..23: mol_type = other DNA
ctggggttgggcgacgacgatgg

SEQ ID NO: 121 - DNA, 23 bp, synthetic construct
  misc_feature 1..23: note = Cypor CRISPR target sequences
  source 1..23: mol_type = other DNA
tactggcgaatgctggaaagggg

SEQ ID NO: 122 - DNA, 23 bp, synthetic construct
  misc_feature 1..23: note = Cypor CRISPR target sequences
  source 1..23: mol_type = other DNA
tcacctggactcctcgccagtgg

SEQ ID NO: 123 - DNA, 23 bp, synthetic construct
  misc_feature 1..23: note = Cypor CRISPR target sequences
  source 1..23: mol_type = other DNA
tggtggccttgaagggcaggcgg

SEQ ID NO: 124 - DNA, 23 bp, synthetic construct
  misc_feature 1..23: note = Cypor CRISPR target sequences
  source 1..23: mol_type = other DNA
taccaacgtgctgtacgagctgg

SEQ ID NO: 125 - DNA, 23 bp, synthetic construct
  misc_feature 1..23: note = Cypor CRISPR target sequences
  source 1..23: mol_type = other DNA
cacctgtggaagttgatcgaagg

SEQ ID NO: 126 - DNA, 23 bp, synthetic construct
  misc_feature 1..23: note = Cypor CRISPR target sequences
  source 1..23: mol_type = other DNA
caacatgggagactcccacgtgg

SEQ ID NO: 127 - DNA, 23 bp, synthetic construct
  misc_feature 1..23: note = Cypor CRISPR target sequences
  source 1..23: mol_type = other DNA
gtactggcgaatgctggaaaggg

SEQ ID NO: 128 - DNA, 23 bp, synthetic construct
  misc_feature 1..23: note = Cypor CRISPR target sequences
  source 1..23: mol_type = other DNA
ggacatgacgacgtccaggtcgg

SEQ ID NO: 129 - DNA, 23 bp, synthetic construct
  misc_feature 1..23: note = Cypor CRISPR target sequences
  source 1..23: mol_type = other DNA
tggaggaggacttcatcacctgg

SEQ ID NO: 130 - DNA, 23 bp, synthetic construct
  misc_feature 1..23: note = Cypor CRISPR target sequences
  source 1..23: mol_type = other DNA
ggtaagctgccaccgcgtgctgg

SEQ ID NO: 131 - DNA, 23 bp, synthetic construct
  misc_feature 1..23: note = Cypor CRISPR target sequences
  source 1..23: mol_type = other DNA
tgtacatgggggagatgggccgg

SEQ ID NO: 132 - DNA, 23 bp, synthetic construct
  misc_feature 1..23: note = Cypor CRISPR target sequences
  source 1..23: mol_type = other DNA
tgcatggccacctacggtgaggg

SEQ ID NO: 133 - DNA, 23 bp, synthetic construct
  misc_feature 1..23: note = Cypor CRISPR target sequences
  source 1..23: mol_type = other DNA
gtatgaatctggggaccacgtgg

SEQ ID NO: 134 - DNA, 23 bp, synthetic construct
  misc_feature 1..23: note = Cypor CRISPR target sequences
  source 1..23: mol_type = other DNA
tggctgcagtcaccaccaaccgg

SEQ ID NO: 135 - DNA, 23 bp, synthetic construct
  misc_feature 1..23: note = Cypor CRISPR target sequences
  source 1..23: mol_type = other DNA
cgacatcgtggctgagctcgggg

SEQ ID NO: 136 - DNA, 23 bp, synthetic construct
  misc_feature 1..23: note = Cypor CRISPR target sequences
  source 1..23: mol_type = other DNA
agtgccggccctcaccttggagg

SEQ ID NO: 137 - DNA, 23 bp, synthetic construct
  misc_feature 1..23: note = Cypor CRISPR target sequences
  source 1..23: mol_type = other DNA
cttgttgatgcggccagccttgg

SEQ ID NO: 138 - DNA, 23 bp, synthetic construct
  misc_feature 1..23: note = Cypor CRISPR target sequences
  source 1..23: mol_type = other DNA
ctggcgagagcagttctggccgg

SEQ ID NO: 139 - DNA, 23 bp, synthetic construct
  misc_feature 1..23: note = Cypor CRISPR target sequences
  source 1..23: mol_type = other DNA
tcaggtcggcctgcagaaacagg

SEQ ID NO: 140 - DNA, 23 bp, synthetic construct
  misc_feature 1..23: note = Cypor CRISPR target sequences
  source 1..23: mol_type = other DNA
agccacgctccctccactcacgg

SEQ ID NO: 141 - DNA, 23 bp, synthetic construct
  misc_feature 1..23: note = Cypor CRISPR target sequences
  source 1..23: mol_type = other DNA
attctgttttcgctcatcgtggg

SEQ ID NO: 142 - DNA, 23 bp, synthetic construct
  misc_feature 1..23: note = Cypor CRISPR target sequences
  source 1..23: mol_type = other DNA
caccaactggctgcgggccaagg

SEQ ID NO: 143 - DNA, 23 bp, synthetic construct
  misc_feature 1..23: note = Cypor CRISPR target sequences
  source 1..23: mol_type = other DNA
gaacaccttctacgacatcgtgg

SEQ ID NO: 144 - DNA, 23 bp, synthetic construct
  misc_feature 1..23: note = Cypor CRISPR target sequences
  source 1..23: mol_type = other DNA
tacgagcacttcaatgccatggg

SEQ ID NO: 145 - DNA, 23 bp, synthetic construct
  misc_feature 1..23: note = Cypor CRISPR target sequences
  source 1..23: mol_type = other DNA
gtacagctcctaagagacacggg

SEQ ID NO: 146 - DNA, 23 bp, synthetic construct
  misc_feature 1..23: note = Cypor CRISPR target sequences
  source 1..23: mol_type = other DNA
tggccacgcccttgttgatgcgg

SEQ ID NO: 147 - DNA, 23 bp, synthetic construct
  misc_feature 1..23: note = Cypor CRISPR target sequences
  source 1..23: mol_type = other DNA
atgcccaggacttctacgactgg

SEQ ID NO: 148 - DNA, 23 bp, synthetic construct
  misc_feature 1..23: note = Cypor CRISPR target sequences
  source 1..23: mol_type = other DNA
caggtgcatgaggtggcgctcgg

SEQ ID NO: 149 - DNA, 23 bp, synthetic construct
  misc_feature 1..23: note = Cypor CRISPR target sequences
  source 1..23: mol_type = other DNA
cgagaaccagaagccgtgagtgg

SEQ ID NO: 150 - DNA, 23 bp, synthetic construct
  misc_feature 1..23: note = Cypor CRISPR target sequences
  source 1..23: mol_type = other DNA
agtcctgggcattgtcggtgggg

SEQ ID NO: 151 - DNA, 23 bp, synthetic construct
  misc_feature 1..23: note = Cypor CRISPR target sequences
  source 1..23: mol_type = other DNA
gggcgttgtcgatctctggcagg

SEQ ID NO: 152 - DNA, 23 bp, synthetic construct
  misc_feature 1..23: note = Cypor CRISPR target sequences
  source 1..23: mol_type = other DNA
ctcacccacagacgtagatgtgg

SEQ ID NO: 153 - DNA, 23 bp, synthetic construct
  misc_feature 1..23: note = Cypor CRISPR target sequences
  source 1..23: mol_type = other DNA
ttcggccaccgcctcggacacgg

SEQ ID NO: 154 - DNA, 23 bp, synthetic construct
  misc_feature 1..23: note = Cypor CRISPR target sequences
  source 1..23: mol_type = other DNA
tacttcttcggccaccgcctcgg

SEQ ID NO: 155 - DNA, 23 bp, synthetic construct
  misc_feature 1..23: note = Cypor CRISPR target sequences
  source 1..23: mol_type = other DNA
acgatgatgttcctcccctaagg

SEQ ID NO: 156 - DNA, 23 bp, synthetic construct
  misc_feature 1..23: note = Cypor CRISPR target sequences
  source 1..23: mol_type = other DNA
aggagctggcgcagttccacagg

SEQ ID NO: 157 - DNA, 23 bp, synthetic construct
  misc_feature 1..23: note = Cypor CRISPR target sequences
  source 1..23: mol_type = other DNA
caaacacctgcgtggacacaggg

SEQ ID NO: 158 - DNA, 23 bp, synthetic construct
  misc_feature 1..23: note = Cypor CRISPR target sequences
  source 1..23: mol_type = other DNA
tggggttgggcgacgacgatggg

SEQ ID NO: 159 - DNA, 23 bp, synthetic construct
  misc_feature 1..23: note = Cypor CRISPR target sequences
  source 1..23: mol_type = other DNA
tctgctctcgtcaaccagctggg

SEQ ID NO: 160 - DNA, 23 bp, synthetic construct
  misc_feature 1..23: note = Cypor CRISPR target sequences
  source 1..23: mol_type = other DNA
ctggttcagcttccggttggtgg

SEQ ID NO: 161 - DNA, 23 bp, synthetic construct
  misc_feature 1..23: note = Cypor CRISPR target sequences
  source 1..23: mol_type = other DNA
gattctgttttcgctcatcgtgg

SEQ ID NO: 162 - DNA, 23 bp, synthetic construct
  misc_feature 1..23: note = Cypor CRISPR target sequences
  source 1..23: mol_type = other DNA
ccggccgtgtgtgaacactttgg

SEQ ID NO: 163 - DNA, 23 bp, synthetic construct
  misc_feature 1..23: note = Cypor CRISPR target sequences
  source 1..23: mol_type = other DNA
ggccgtgtgtgaacactttgggg

SEQ ID NO: 164 - DNA, 23 bp, synthetic construct
  misc_feature 1..23: note = Cypor CRISPR target sequences
  source 1..23: mol_type = other DNA
ctccctggacgtgtggagctagg

SEQ ID NO: 165 - DNA, 23 bp, synthetic construct
  misc_feature 1..23: note = Cypor CRISPR target sequences
  source 1..23: mol_type = other DNA
gggccgtgcggtaggacgtaggg

SEQ ID NO: 166 - DNA, 23 bp, synthetic construct
  misc_feature 1..23: note = Cypor CRISPR target sequences
  source 1..23: mol_type = other DNA
gactgtggcactcaccatccagg

SEQ ID NO: 167 - DNA, 23 bp, synthetic construct
  misc_feature 1..23: note = Cypor CRISPR target sequences
  source 1..23: mol_type = other DNA
ttaggagctgtacctgagctggg

SEQ ID NO: 168 - DNA, 23 bp, synthetic construct
  misc_feature 1..23: note = Cypor CRISPR target sequences
  source 1..23: mol_type = other DNA
cggccagaactgctctcgccagg

SEQ ID NO: 169 - DNA, 23 bp, synthetic construct
  misc_feature 1..23: note = Cypor CRISPR target sequences
  source 1..23: mol_type = other DNA
gcccctagctccacacgtccagg

SEQ ID NO: 170 - DNA, 23 bp, synthetic construct
  misc_feature 1..23: note = Cypor CRISPR target sequences
  source 1..23: mol_type = other DNA
gaccaagggccgctactccctgg

SEQ ID NO: 171 - DNA, 23 bp, synthetic construct
  misc_feature 1..23: note = Cypor CRISPR target sequences
  source 1..23: mol_type = other DNA
ctccttggcccgcagccagttgg

SEQ ID NO: 172 - DNA, 23 bp, synthetic construct
  misc_feature 1..23: note = Cypor CRISPR target sequences
  source 1..23: mol_type = other DNA
tgcccgcccgtctcaccttgtgg

SEQ ID NO: 173 - DNA, 23 bp, synthetic construct
  misc_feature 1..23: note = Cypor CRISPR target sequences
  source 1..23: mol_type = other DNA
tgaggtggcgctcggttccctgg

SEQ ID NO: 174 - DNA, 23 bp, synthetic construct
  misc_feature 1..23: note = Cypor CRISPR target sequences
  source 1..23: mol_type = other DNA
accagggcgttgtcgatctctgg

SEQ ID NO: 175 - DNA, 23 bp, synthetic construct
  misc_feature 1..23: note = Cypor CRISPR target sequences
  source 1..23: mol_type = other DNA
ggatgaggactacctgtaccggg

SEQ ID NO: 176 - DNA, 23 bp, synthetic construct
  misc_feature 1..23: note = Cypor CRISPR target sequences
  source 1..23: mol_type = other DNA
tcaccttgtgggactgctcccgg

SEQ ID NO: 177 - DNA, 23 bp, synthetic construct
  misc_feature 1..23: note = Cypor CRISPR target sequences
  source 1..23: mol_type = other DNA
atctcacccccgtgtctcttagg

SEQ ID NO: 178 - DNA, 23 bp, synthetic construct
  misc_feature 1..23: note = Cypor CRISPR target sequences
  source 1..23: mol_type = other DNA
catggagcacgcgcaggcggtgg

SEQ ID NO: 179 - DNA, 23 bp, synthetic construct
  misc_feature 1..23: note = Cypor CRISPR target sequences
  source 1..23: mol_type = other DNA
gcgcaccttgccctcgccggagg

SEQ ID NO: 180 - DNA, 23 bp, synthetic construct
  misc_feature 1..23: note = Cypor CRISPR target sequences
  source 1..23: mol_type = other DNA
gatggcctcctcctccggcgagg

SEQ ID NO: 181 - DNA, 23 bp, synthetic construct
  misc_feature 1..23: note = Cypor CRISPR target sequences
  source 1..23: mol_type = other DNA
tcgatggggggccgcagggacgg

SEQ ID NO: 182 - DNA, 23 bp, synthetic construct
  misc_feature 1..23: note = Cypor CRISPR target sequences
  source 1..23: mol_type = other DNA
ggagaacggcggccgtgcgctgg

SEQ ID NO: 183 - DNA, 23 bp, synthetic construct
  misc_feature 1..23: note = Cypor CRISPR target sequences
  source 1..23: mol_type = other DNA
gaagggtgccaccccggtgccgg

SEQ ID NO: 184 - DNA, 23 bp, synthetic construct
  misc_feature 1..23: note = Cypor CRISPR target sequences
  source 1..23: mol_type = other DNA
gcccaccgctacgggatgcgagg

SEQ ID NO: 185 - DNA, 23 bp, synthetic construct
  misc_feature 1..23: note = Cypor CRISPR target sequences
  source 1..23: mol_type = other DNA
gggtgagcgcaccgtccctgtgg

SEQ ID NO: 186 - DNA, 23 bp, synthetic construct
  misc_feature 1..23: note = Cypor CRISPR target sequences
  source 1..23: mol_type = other DNA
cgacgtccaggtcggcacccagg

SEQ ID NO: 187 - DNA, 23 bp, synthetic construct
  misc_feature 1..23: note = Cypor CRISPR target sequences
  source 1..23: mol_type = other DNA
tgcgctcacccagctcaacgtgg

SEQ ID NO: 188 - DNA, 23 bp, synthetic construct
  misc_feature 1..23: note = Cypor CRISPR target sequences
  source 1..23: mol_type = other DNA
caaggctggccgcatcaacaagg

SEQ ID NO: 189 - DNA, 23 bp, synthetic construct
  misc_feature 1..23: note = Cypor CRISPR target sequences
  source 1..23: mol_type = other DNA
gttttctgcatggccacctacgg

SEQ ID NO: 190 - DNA, 23 bp, synthetic construct
  misc_feature 1..23: note = Cypor CRISPR target sequences
  source 1..23: mol_type = other DNA
cttaccaggtcatactcctcagg

SEQ ID NO: 191 - DNA, 23 bp, synthetic construct
  misc_feature 1..23: note = Cypor CRISPR target sequences
  source 1..23: mol_type = other DNA
ctctgtgcacatctgtgcggtgg

SEQ ID NO: 192 - DNA, 23 bp, synthetic construct
  misc_feature 1..23: note = Cypor CRISPR target sequences
  source 1..23: mol_type = other DNA
gcgtggtggccttgaagggcagg

SEQ ID NO: 193 - DNA, 23 bp, synthetic construct
  misc_feature 1..23: note = Cypor CRISPR target sequences
  source 1..23: mol_type = other DNA
tcatcctccaaggtgagggccgg

SEQ ID NO: 194 - DNA, 23 bp, synthetic construct
  misc_feature 1..23: note = Cypor CRISPR target sequences
  source 1..23: mol_type = other DNA
ctgcatggccacctacggtgagg

SEQ ID NO: 195 - DNA, 23 bp, synthetic construct
  misc_feature 1..23: note = Cypor CRISPR target sequences
  source 1..23: mol_type = other DNA
accaaccggaagctgaaccaggg

SEQ ID NO: 196 - DNA, 23 bp, synthetic construct
  misc_feature 1..23: note = Cypor CRISPR target sequences
  source 1..23: mol_type = other DNA
accccagctcaaagatgcgctgg

SEQ ID NO: 197 - DNA, 23 bp, synthetic construct
  misc_feature 1..23: note = Cypor CRISPR target sequences
  source 1..23: mol_type = other DNA
tctgaattttggtgaactcgggg

SEQ ID NO: 198 - DNA, 23 bp, synthetic construct
  misc_feature 1..23: note = Cypor CRISPR target sequences
  source 1..23: mol_type = other DNA
ctcccgggagcagtcccacaagg

SEQ ID NO: 199 - DNA, 23 bp, synthetic construct
  misc_feature 1..23: note = Cypor CRISPR target sequences
  source 1..23: mol_type = other DNA
cttaggagctgtacctgagctgg

SEQ ID NO: 200 - DNA, 23 bp, synthetic construct
  misc_feature 1..23: note = Cypor CRISPR target sequences
  source 1..23: mol_type = other DNA
gttgacgagagcagagtcgttgg

SEQ ID NO: 201 - DNA, 23 bp, synthetic construct
  misc_feature 1..23: note = Cypor CRISPR target sequences
  source 1..23: mol_type = other DNA
tcagggacatgacgacgtccagg

SEQ ID NO: 202 - DNA, 23 bp, synthetic construct
  misc_feature 1..23: note = Cypor CRISPR target sequences
  source 1..23: mol_type = other DNA
aaggagcctgccggggagaacgg

SEQ ID NO: 203 - DNA, 23 bp, synthetic construct
  misc_feature 1..23: note = Cypor CRISPR target sequences
  source 1..23: mol_type = other DNA
caatgccatgggcaagtacgtgg

SEQ ID NO: 204 - DNA, 23 bp, synthetic construct
  misc_feature 1..23: note = Cypor CRISPR target sequences
  source 1..23: mol_type = other DNA
accggggtggcacccttcatagg

SEQ ID NO: 205 - DNA, 23 bp, synthetic construct
  misc_feature 1..23: note = Cypor CRISPR target sequences
  source 1..23: mol_type = other DNA
cgccagctcgtacagcacgttgg

SEQ ID NO: 206 - DNA, 23 bp, synthetic construct
  misc_feature 1..23: note = Cypor CRISPR target sequences
  source 1..23: mol_type = other DNA
acgacatcgtggctgagctcggg

SEQ ID NO: 207 - DNA, 23 bp, synthetic construct
  misc_feature 1..23: note = Cypor CRISPR target sequences
  source 1..23: mol_type = other DNA
cgagagcagagtcgttggctggg

SEQ ID NO: 208 - DNA, 23 bp, synthetic construct
  misc_feature 1..23: note = Cypor CRISPR target sequences
  source 1..23: mol_type = other DNA
ggtagtaggtgagggccgtgcgg

SEQ ID NO: 209 - DNA, 23 bp, synthetic construct
  misc_feature 1..23: note = Cypor CRISPR target sequences
  source 1..23: mol_type = other DNA
agcaagaccgagagcacctgtgg

SEQ ID NO: 210 - DNA, 23 bp, synthetic construct
  misc_feature 1..23: note = Cypor CRISPR target sequences
  source 1..23: mol_type = other DNA
cgcaagatggcctcctcctccgg

SEQ ID NO: 211 - DNA, 23 bp, synthetic construct
  misc_feature 1..23: note = Cypor CRISPR target sequences
  source 1..23: mol_type = other DNA
cggtggagctggtgtccacgtgg

SEQ ID NO: 212 - DNA, 23 bp, synthetic construct
  misc_feature 1..23: note = Cypor CRISPR target sequences
  source 1..23: mol_type = other DNA
cagccagtcgtagaagtcctggg

SEQ ID NO: 213 - DNA, 23 bp, synthetic construct
  misc_feature 1..23: note = Cypor CRISPR target sequences
  source 1..23: mol_type = other DNA
tcctggatgaagcctatgaaggg

SEQ ID NO: 214 - DNA, 23 bp, synthetic construct
  misc_feature 1..23: note = Cypor CRISPR target sequences
  source 1..23: mol_type = other DNA
acgagagcagagtcgttggctgg

SEQ ID NO: 215 - DNA, 23 bp, synthetic construct
  misc_feature 1..23: note = Cypor CRISPR target sequences
  source 1..23: mol_type = other DNA
gacaagcggctggagcagctcgg

SEQ ID NO: 216 - DNA, 23 bp, synthetic construct
  misc_feature 1..23: note = Cypor CRISPR target sequences
  source 1..23: mol_type = other DNA
ggatggccaggatgtgcctccgg

SEQ ID NO: 217 - DNA, 23 bp, synthetic construct
  misc_feature 1..23: note = Cypor CRISPR target sequences
  source 1..23: mol_type = other DNA
ccagttccgcctgcccttcaagg

SEQ ID NO: 218 - DNA, 23 bp, synthetic construct
  misc_feature 1..23: note = Cypor CRISPR target sequences
  source 1..23: mol_type = other DNA
gcagccagtcgtagaagtcctgg

SEQ ID NO: 219 - DNA, 23 bp, synthetic construct
  misc_feature 1..23: note = Cypor CRISPR target sequences
  source 1..23: mol_type = other DNA
agctcacacaggtggtcgatggg

SEQ ID NO: 220 - DNA, 23 bp, synthetic construct
  misc_feature 1..23: note = Cypor CRISPR target sequences
  source 1..23: mol_type = other DNA
gctctcggtcttgctttagcagg

SEQ ID NO: 221 - DNA, 23 bp, synthetic construct
  misc_feature 1..23: note = Cypor CRISPR target sequences
  source 1..23: mol_type = other DNA
tccacgcaggtgtttggtcttgg

SEQ ID NO: 222 - DNA, 23 bp, synthetic construct
  misc_feature 1..23: note = Cypor CRISPR target sequences
  source 1..23: mol_type = other DNA
ccacgcaggtgtttggtcttggg

SEQ ID NO: 223 - DNA, 23 bp, synthetic construct
  misc_feature 1..23: note = Cypor CRISPR target sequences
  source 1..23: mol_type = other DNA
actcaccatccaggttgttcagg

SEQ ID NO: 224 - DNA, 23 bp, synthetic construct
  misc_feature 1..23: note = Cypor CRISPR target sequences
  source 1..23: mol_type = other DNA
tcaacttccacaggtgctctcgg

SEQ ID NO: 225 - DNA, 23 bp, synthetic construct
  misc_feature 1..23: note = Cypor CRISPR target sequences
  source 1..23: mol_type = other DNA
cacctccttgcctgcgtggcagg

SEQ ID NO: 226 - DNA, 23 bp, synthetic construct
  misc_feature 1..23: note = Cypor CRISPR target sequences
  source 1..23: mol_type = other DNA
tgcagccaggaacggattcttgg

SEQ ID NO: 227 - DNA, 23 bp, synthetic construct
  misc_feature 1..23: note = Cypor CRISPR target sequences
  source 1..23: mol_type = other DNA
cggctgccgccgctcggatgagg

SEQ ID NO: 228 - DNA, 23 bp, synthetic construct
  misc_feature 1..23: note = Cypor CRISPR target sequences
  source 1..23: mol_type = other DNA
ggccaccgcctcggacacggtgg

SEQ ID NO: 229 - DNA, 23 bp, synthetic construct
  misc_feature 1..23: note = Cypor CRISPR target sequences
  source 1..23: mol_type = other DNA
cttgcctgcgtggcaggggccgg

SEQ ID NO: 230 - DNA, 23 bp, synthetic construct
  misc_feature 1..23: note = Cypor CRISPR target sequences
  source 1..23: mol_type = other DNA
cgcacggccgccgttctccccgg

SEQ ID NO: 231 - DNA, 23 bp, synthetic construct
  misc_feature 1..23: note = Cypor CRISPR target sequences
  source 1..23: mol_type = other DNA
caccgcctgcgcgtgctccatgg

SEQ ID NO: 232 - DNA, 23 bp, synthetic construct
  misc_feature 1..23: note = Cypor CRISPR target sequences
  source 1..23: mol_type = other DNA
gctgaaaagagatacttcttcgg

SEQ ID NO: 233 - DNA, 23 bp, synthetic construct
  misc_feature 1..23: note = Cypor CRISPR target sequences
  source 1..23: mol_type = other DNA
tgcgggccaaggagcctgccggg

SEQ ID NO: 234 - DNA, 23 bp, synthetic construct
  misc_feature 1..23: note = Cypor CRISPR target sequences
  source 1..23: mol_type = other DNA
cagctccaccgtgtccgaggcgg

SEQ ID NO: 235 - DNA, 23 bp, synthetic construct
  misc_feature 1..23: note = Cypor CRISPR target sequences
  source 1..23: mol_type = other DNA
ggtggggtctccctcaccgtagg

SEQ ID NO: 236 - DNA, 23 bp, synthetic construct
  misc_feature 1..23: note = Cypor CRISPR target sequences
  source 1..23: mol_type = other DNA
gtggagtacgagaccaaggctgg

SEQ ID NO: 237 - DNA, 23 bp, synthetic construct
  misc_feature 1..23: note = Cypor CRISPR target sequences
  source 1..23: mol_type = other DNA
agcggtgggcgtccttggacagg

SEQ ID NO: 238 - DNA, 23 bp, synthetic construct
  misc_feature 1..23: note = Cypor CRISPR target sequences
  source 1..23: mol_type = other DNA
caccttgtgggactgctcccggg

SEQ ID NO: 239 - DNA, 23 bp, synthetic construct
  misc_feature 1..23: note = Cypor CRISPR target sequences
  source 1..23: mol_type = other DNA
caaaatcctgggtgccgacctgg

SEQ ID NO: 240 - DNA, 23 bp, synthetic construct
  misc_feature 1..23: note = Cypor CRISPR target sequences
  source 1..23: mol_type = other DNA
ggccatggagcacgcgcaggcgg

SEQ ID NO: 241 - DNA, 23 bp, synthetic construct
  misc_feature 1..23: note = Cypor CRISPR target sequences
  source 1..23: mol_type = other DNA
acagctcctaagagacacggggg

SEQ ID NO: 242 - DNA, 23 bp, synthetic construct
  misc_feature 1..23: note = Cypor CRISPR target sequences
  source 1..23: mol_type = other DNA
ctgccacgcaggcaaggaggtgg

SEQ ID NO: 243 - DNA, 23 bp, synthetic construct
  misc_feature 1..23: note = Cypor CRISPR target sequences
  source 1..23: mol_type = other DNA
tgaggactacctgtaccgggagg

SEQ ID NO: 244 - DNA, 23 bp, synthetic construct
  misc_feature 1..23: note = Cypor CRISPR target sequences
  source 1..23: mol_type = other DNA
aggtgctggacgtagacctgggg

SEQ ID NO: 245 - DNA, 23 bp, synthetic construct
  misc_feature 1..23: note = Cypor CRISPR target sequences
  source 1..23: mol_type = other DNA
cccagcgcatctttgagctgggg

SEQ ID NO: 246 - DNA, 23 bp, synthetic construct
  misc_feature 1..23: note = Cypor CRISPR target sequences
  source 1..23: mol_type = other DNA
tacagctcctaagagacacgggg

SEQ ID NO: 247 - DNA, 23 bp, synthetic construct
  misc_feature 1..23: note = Cypor CRISPR target sequences
  source 1..23: mol_type = other DNA
ggtggagctggtgtccacgtggg

SEQ ID NO: 248 - DNA, 23 bp, synthetic construct
  misc_feature 1..23: note = Cypor CRISPR target sequences
  source 1..23: mol_type = other DNA
ggccatgcagaaaaccaccaggg

SEQ ID NO: 249 - DNA, 23 bp, synthetic construct
  misc_feature 1..23: note = Cypor CRISPR target sequences
  source 1..23: mol_type = other DNA
gtggtgactgcagccaggaacgg

SEQ ID NO: 250 - DNA, 23 bp, synthetic construct
  misc_feature 1..23: note = Cypor CRISPR target sequences
  source 1..23: mol_type = other DNA
agaccccaccgacaatgcccagg

SEQ ID NO: 251 - DNA, 23 bp, synthetic construct
  misc_feature 1..23: note = Cypor CRISPR target sequences
  source 1..23: mol_type = other DNA
gtgggactgctcccgggagaagg

SEQ ID NO: 252 - DNA, 23 bp, synthetic construct
  misc_feature 1..23: note = Cypor CRISPR target sequences
  source 1..23: mol_type = other DNA
tcacccacagacgtagatgtggg

SEQ ID NO: 253 - DNA, 23 bp, synthetic construct
  misc_feature 1..23: note = Cypor CRISPR target sequences
  source 1..23: mol_type = other DNA
ggagctggcgcagttccacaggg

SEQ ID NO: 254 - DNA, 23 bp, synthetic construct
  misc_feature 1..23: note = Cypor CRISPR target sequences
  source 1..23: mol_type = other DNA
gccatgttccgtgcatccctggg

SEQ ID NO: 255 - DNA, 23 bp, synthetic construct
  misc_feature 1..23: note = Cypor CRISPR target sequences
  source 1..23: mol_type = other DNA
ctttctgaagaggaaccagtagg

SEQ ID NO: 256 - DNA, 23 bp, synthetic construct
  misc_feature 1..23: note = Cypor CRISPR target sequences
  source 1..23: mol_type = other DNA
cgcctcatcctccaaggtgaggg

SEQ ID NO: 257 - DNA, 23 bp, synthetic construct
  misc_feature 1..23: note = Cypor CRISPR target sequences
  source 1..23: mol_type = other DNA
cttgtccacgtacttgcccatgg

SEQ ID NO: 258 - DNA, 23 bp, synthetic construct
  misc_feature 1..23: note = Cypor CRISPR target sequences
  source 1..23: mol_type = other DNA
ctctggcaggctgctcaggtcgg

SEQ ID NO: 259 - DNA, 23 bp, synthetic construct
  misc_feature 1..23: note = Cypor CRISPR target sequences
  source 1..23: mol_type = other DNA
gctgcgacagcagggtgagtggg

SEQ ID NO: 260 - DNA, 23 bp, synthetic construct
  misc_feature 1..23: note = Cypor CRISPR target sequences
  source 1..23: mol_type = other DNA
ctcctggatgaagcctatgaagg

SEQ ID NO: 261 - DNA, 23 bp, synthetic construct
  misc_feature 1..23: note = Cypor CRISPR target sequences
  source 1..23: mol_type = other DNA
catggcattgaagtgctcgtagg

SEQ ID NO: 262 - DNA, 23 bp, synthetic construct
  misc_feature 1..23: note = Cypor CRISPR target sequences
  source 1..23: mol_type = other DNA
tgcaggactgcccgtccctgcgg

SEQ ID NO: 263 - DNA, 23 bp, synthetic construct
  misc_feature 1..23: note = Cypor CRISPR target sequences
  source 1..23: mol_type = other DNA
ttggcatcaaaggggctgggtgg

SEQ ID NO: 264 - DNA, 23 bp, synthetic construct
  misc_feature 1..23: note = Cypor CRISPR target sequences
  source 1..23: mol_type = other DNA
gcccagggatgcacggaacatgg

SEQ ID NO: 265 - DNA, 23 bp, synthetic construct
  misc_feature 1..23: note = Cypor CRISPR target sequences
  source 1..23: mol_type = other DNA
tggagtagtagcgggcctgcagg

SEQ ID NO: 266 - DNA, 23 bp, synthetic construct
  misc_feature 1..23: note = Cypor CRISPR target sequences
  source 1..23: mol_type = other DNA
gatgaagtcctcctccaagctgg

SEQ ID NO: 267 - DNA, 23 bp, synthetic construct
  misc_feature 1..23: note = Cypor CRISPR target sequences
  source 1..23: mol_type = other DNA
tgccacgcaggcaaggaggtggg

SEQ ID NO: 268 - DNA, 23 bp, synthetic construct
  misc_feature 1..23: note = Cypor CRISPR target sequences
  source 1..23: mol_type = other DNA
cgtcatgtccctgaacaacctgg

SEQ ID NO: 269 - DNA, 23 bp, synthetic construct
  misc_feature 1..23: note = Cypor CRISPR target sequences
  source 1..23: mol_type = other DNA
cgcccagcgcatctttgagctgg

SEQ ID NO: 270 - DNA, 23 bp, synthetic construct
  misc_feature 1..23: note = Cypor CRISPR target sequences
  source 1..23: mol_type = other DNA
ggcgtccttggacaggcggttgg

SEQ ID NO: 271 - DNA, 23 bp, synthetic construct
  misc_feature 1..23: note = Cypor CRISPR target sequences
  source 1..23: mol_type = other DNA
gcggcgggttggtgatgtccagg

SEQ ID NO: 272 - DNA, 23 bp, synthetic construct
  misc_feature 1..23: note = Cypor CRISPR target sequences
  source 1..23: mol_type = other DNA
gacagacgtggatctctctgggg

SEQ ID NO: 273 - DNA, 23 bp, synthetic construct
  misc_feature 1..23: note = Cypor CRISPR target sequences
  source 1..23: mol_type = other DNA
ctgcgacagcagggtgagtgggg

SEQ ID NO: 274 - DNA, 23 bp, synthetic construct
  misc_feature 1..23: note = Cypor CRISPR target sequences
  source 1..23: mol_type = other DNA
ggtcttgctttagcaggtgctgg

SEQ ID NO: 275 - DNA, 23 bp, synthetic construct
  misc_feature 1..23: note = Cypor CRISPR target sequences
  source 1..23: mol_type = other DNA
tccctggttcagcttccggttgg

SEQ ID NO: 276 - DNA, 23 bp, synthetic construct
  misc_feature 1..23: note = Cypor CRISPR target sequences
  source 1..23: mol_type = other DNA
accacgcctgtcatcatggtggg

SEQ ID NO: 277 - DNA, 23 bp, synthetic construct
  misc_feature 1..23: note = Cypor CRISPR target sequences
  source 1..23: mol_type = other DNA
cagagttggggtggacctgtggg

SEQ ID NO: 278 - DNA, 23 bp, synthetic construct
  misc_feature 1..23: note = Cypor CRISPR target sequences
  source 1..23: mol_type = other DNA
gctcaacgtggccttctcccggg

SEQ ID NO: 279 - DNA, 23 bp, synthetic construct
  misc_feature 1..23: note = Cypor CRISPR target sequences
  source 1..23: mol_type = other DNA
cgcacagatgtgcacagagttgg

SEQ ID NO: 280 - DNA, 23 bp, synthetic construct
  misc_feature 1..23: note = Cypor CRISPR target sequences
  source 1..23: mol_type = other DNA
cgagcgccacctcatgcacctgg

SEQ ID NO: 281 - DNA, 23 bp, synthetic construct
  misc_feature 1..23: note = Cypor CRISPR target sequences
  source 1..23: mol_type = other DNA
tccccagattcatacctggaggg

SEQ ID NO: 282 - DNA, 23 bp, synthetic construct
  misc_feature 1..23: note = Cypor CRISPR target sequences
  source 1..23: mol_type = other DNA
tgaagaggaaccagtaggttagg

SEQ ID NO: 283 - DNA, 23 bp, synthetic construct
  misc_feature 1..23: note = Cypor CRISPR target sequences
  source 1..23: mol_type = other DNA
ctccctccaggtatgaatctggg

SEQ ID NO: 284 - DNA, 23 bp, synthetic construct
  misc_feature 1..23: note = Cypor CRISPR target sequences
  source 1..23: mol_type = other DNA
gttggtgatgtccaggtagtagg

SEQ ID NO: 285 - DNA, 23 bp, synthetic construct
  misc_feature 1..23: note = Cypor CRISPR target sequences
  source 1..23: mol_type = other DNA
tccctggacgtgtggagctaggg

SEQ ID NO: 286 - DNA, 23 bp, synthetic construct
  misc_feature 1..23: note = Cypor CRISPR target sequences
  source 1..23: mol_type = other DNA
agctcaacgtggccttctcccgg

SEQ ID NO: 287 - DNA, 23 bp, synthetic construct
  misc_feature 1..23: note = Cypor CRISPR target sequences
  source 1..23: mol_type = other DNA
agatgcggccaaggtgtacatgg

SEQ ID NO: 288 - DNA, 23 bp, synthetic construct
  misc_feature 1..23: note = Cypor CRISPR target sequences
  source 1..23: mol_type = other DNA
ctcaccatccaggttgttcaggg

SEQ ID NO: 289 - DNA, 23 bp, synthetic construct
  misc_feature 1..23: note = Cypor CRISPR target sequences
  source 1..23: mol_type = other DNA
cggccgtgtgtgaacactttggg

SEQ ID NO: 290 - DNA, 23 bp, synthetic construct
  misc_feature 1..23: note = Cypor CRISPR target sequences
  source 1..23: mol_type = other DNA
cggggccatggagcacgcgcagg

SEQ ID NO: 291 - DNA, 23 bp, synthetic construct
  misc_feature 1..23: note = Cypor CRISPR target sequences
  source 1..23: mol_type = other DNA
ctccaccgtgtccgaggcggtgg

SEQ ID NO: 292 - DNA, 23 bp, synthetic construct
  misc_feature 1..23: note = Cypor CRISPR target sequences
  source 1..23: mol_type = other DNA
catggtgggccccggcaccgggg

SEQ ID NO: 293 - DNA, 23 bp, synthetic construct
  misc_feature 1..23: note = Cypor CRISPR target sequences
  source 1..23: mol_type = other DNA
ctgcgggccaaggagcctgccgg

SEQ ID NO: 294 - DNA, 23 bp, synthetic construct
  misc_feature 1..23: note = Cypor CRISPR target sequences
  source 1..23: mol_type = other DNA
gcgccagctcctcccggtacagg

SEQ ID NO: 295 - DNA, 23 bp, synthetic construct
  misc_feature 1..23: note = Cypor CRISPR target sequences
  source 1..23: mol_type = other DNA
agtatctcttttcagcatgacgg

SEQ ID NO: 296 - DNA, 23 bp, synthetic construct
  misc_feature 1..23: note = Cypor CRISPR target sequences
  source 1..23: mol_type = other DNA
tgtctgaattttggtgaactcgg

SEQ ID NO: 297 - DNA, 23 bp, synthetic construct
  misc_feature 1..23: note = Cypor CRISPR target sequences
  source 1..23: mol_type = other DNA
cgtgtgtgaacactttggggtgg

SEQ ID NO: 298 - DNA, 23 bp, synthetic construct
  misc_feature 1..23: note = Cypor CRISPR target sequences
  source 1..23: mol_type = other DNA
gtgtctgccttccttaggggagg

SEQ ID NO: 299 - DNA, 23 bp, synthetic construct
  misc_feature 1..23: note = Cypor CRISPR target sequences
  source 1..23: mol_type = other DNA
tgtggaccacaagctcgtactgg

SEQ ID NO: 300 - DNA, 23 bp, synthetic construct
  misc_feature 1..23: note = Cypor CRISPR target sequences
  source 1..23: mol_type = other DNA
aaccagctgggcaaaatcctggg

SEQ ID NO: 301 - DNA, 23 bp, synthetic construct
  misc_feature 1..23: note = Cypor CRISPR target sequences
  source 1..23: mol_type = other DNA
gtgcccacatctacgtctgtggg

SEQ ID NO: 302 - DNA, 23 bp, synthetic construct
  misc_feature 1..23: note = Cypor CRISPR target sequences
  source 1..23: mol_type = other DNA
caccaaccggaagctgaaccagg

SEQ ID NO: 303 - DNA, 23 bp, synthetic construct
  misc_feature 1..23: note = Cypor CRISPR target sequences
  source 1..23: mol_type = other DNA
attgcacacttttgtcttgcagg

SEQ ID NO: 304 - DNA, 23 bp, synthetic construct
  misc_feature 1..23: note = Cypor CRISPR target sequences
  source 1..23: mol_type = other DNA
tgatgttcctcccctaaggaagg

SEQ ID NO: 305 - DNA, 23 bp, synthetic construct
  misc_feature 1..23: note = Cypor CRISPR target sequences
  source 1..23: mol_type = other DNA
caggtgctggacgtagacctggg

SEQ ID NO: 306 - DNA, 23 bp, synthetic construct
  misc_feature 1..23: note = Cypor CRISPR target sequences
  source 1..23: mol_type = other DNA
ctggagggagatgtgaggctcgg

SEQ ID NO: 307 - DNA, 23 bp, synthetic construct
  misc_feature 1..23: note = Cypor CRISPR target sequences
  source 1..23: mol_type = other DNA
ctcccagacggggactgcagagg

SEQ ID NO: 308 - DNA, 23 bp, synthetic construct
  misc_feature 1..23: note = Cypor CRISPR target sequences
  source 1..23: mol_type = other DNA
catctcccccatgtacaccttgg

SEQ ID NO: 309 - DNA, 23 bp, synthetic construct
  misc_feature 1..23: note = Cypor CRISPR target sequences
  source 1..23: mol_type = other DNA
cggcctgcagaaacagggacagg

SEQ ID NO: 310 - DNA, 23 bp, synthetic construct
  misc_feature 1..23: note = Cypor CRISPR target sequences
  source 1..23: mol_type = other DNA
ctcacacaggtggtcgatggggg

SEQ ID NO: 311 - DNA, 23 bp, synthetic construct
  misc_feature 1..23: note = Cypor CRISPR target sequences
  source 1..23: mol_type = other DNA
caccagctccaccgtgtccgagg

SEQ ID NO: 312 - DNA, 23 bp, synthetic construct
  misc_feature 1..23: note = Cypor CRISPR target sequences
  source 1..23: mol_type = other DNA
gatggccaggatgtgcctccggg

SEQ ID NO: 313 - DNA, 23 bp, synthetic construct
  misc_feature 1..23: note = Cypor CRISPR target sequences
  source 1..23: mol_type = other DNA
tgtccaattccaggtgcatgagg

SEQ ID NO: 314 - DNA, 23 bp, synthetic construct
  misc_feature 1..23: note = Cypor CRISPR target sequences
  source 1..23: mol_type = other DNA
acatctcggactccaaaatcagg

SEQ ID NO: 315 - DNA, 23 bp, synthetic construct
  misc_feature 1..23: note = Cypor CRISPR target sequences
  source 1..23: mol_type = other DNA
gtttgccaaccgcctgtccaagg

SEQ ID NO: 316 - DNA, 23 bp, synthetic construct
  misc_feature 1..23: note = Cypor CRISPR target sequences
  source 1..23: mol_type = other DNA
agtccgagatgtccaattccagg

SEQ ID NO: 317 - DNA, 23 bp, synthetic construct
  misc_feature 1..23: note = Cypor CRISPR target sequences
  source 1..23: mol_type = other DNA
ggctgcgacagcagggtgagtgg

SEQ ID NO: 318 - DNA, 23 bp, synthetic construct
  misc_feature 1..23: note = Cypor CRISPR target sequences
  source 1..23: mol_type = other DNA
aggacgtagggcacgggaatggg

SEQ ID NO: 319 - DNA, 23 bp, synthetic construct
  misc_feature 1..23: note = Cypor CRISPR target sequences
  source 1..23: mol_type = other DNA
cgcatctttgagctggggttggg

SEQ ID NO: 320 - DNA, 23 bp, synthetic construct
  misc_feature 1..23: note = Cypor CRISPR target sequences
  source 1..23: mol_type = other DNA
ccacctcatgcacctggaattgg

SEQ ID NO: 321 - DNA, 23 bp, synthetic construct
  misc_feature 1..23: note = Cypor CRISPR target sequences
  source 1..23: mol_type = other DNA
ctccatcgcctcatcctccaagg

SEQ ID NO: 322 - DNA, 23 bp, synthetic construct
  misc_feature 1..23: note = Cypor CRISPR target sequences
  source 1..23: mol_type = other DNA
agagttggggtggacctgtgggg

SEQ ID NO: 323 - DNA, 23 bp, synthetic construct
  misc_feature 1..23: note = Cypor CRISPR target sequences
  source 1..23: mol_type = other DNA
gcacatctgtgcggtggttgtgg

SEQ ID NO: 324 - DNA, 23 bp, synthetic construct
  misc_feature 1..23: note = Cypor CRISPR target sequences
  source 1..23: mol_type = other DNA
gccgctactccctggacgtgtgg

SEQ ID NO: 325 - DNA, 23 bp, synthetic construct
  misc_feature 1..23: note = Cypor CRISPR target sequences
  source 1..23: mol_type = other DNA
ggccatgttccgtgcatccctgg

SEQ ID NO: 326 - DNA, 23 bp, synthetic construct
  misc_feature 1..23: note = Cypor CRISPR target sequences
  source 1..23: mol_type = other DNA
gttctccccggcaggctccttgg

SEQ ID NO: 327 - DNA, 23 bp, synthetic construct
  misc_feature 1..23: note = Cypor CRISPR target sequences
  source 1..23: mol_type = other DNA
actcctctgcagtccccgtctgg

SEQ ID NO: 328 - DNA, 23 bp, synthetic construct
  misc_feature 1..23: note = Cypor CRISPR target sequences
  source 1..23: mol_type = other DNA
gagacagacgtggatctctctgg

SEQ ID NO: 329 - DNA, 23 bp, synthetic construct
  misc_feature 1..23: note = Cypor CRISPR target sequences
  source 1..23: mol_type = other DNA
gagcctgccggggagaacggcgg

SEQ ID NO: 330 - DNA, 23 bp, synthetic construct
  misc_feature 1..23: note = Cypor CRISPR target sequences
  source 1..23: mol_type = other DNA
agggtgccaccccggtgccgggg

SEQ ID NO: 331 - DNA, 23 bp, synthetic construct
  misc_feature 1..23: note = Cypor CRISPR target sequences
  source 1..23: mol_type = other DNA
agcggcccctgtgtccacgcagg

SEQ ID NO: 332 - DNA, 23 bp, synthetic construct
  misc_feature 1..23: note = Cypor CRISPR target sequences
  source 1..23: mol_type = other DNA
gcgggcctggctgcgacagcagg

SEQ ID NO: 333 - DNA, 23 bp, synthetic construct
  misc_feature 1..23: note = Cypor CRISPR target sequences
  source 1..23: mol_type = other DNA
cgcctcggagccctcggagcagg

SEQ ID NO: 334 - DNA, 23 bp, synthetic construct
  misc_feature 1..23: note = Cypor CRISPR target sequences
  source 1..23: mol_type = other DNA
gtggtcgatggggggccgcaggg

SEQ ID NO: 335 - DNA, 23 bp, synthetic construct
  misc_feature 1..23: note = Cypor CRISPR target sequences
  source 1..23: mol_type = other DNA
tcatggtgggccccggcaccggg

SEQ ID NO: 336 - DNA, 23 bp, synthetic construct
  misc_feature 1..23: note = Cypor CRISPR target sequences
  source 1..23: mol_type = other DNA
ggcagtgccggccctcaccttgg

SEQ ID NO: 337 - DNA, 23 bp, synthetic construct
  misc_feature 1..23: note = Cypor CRISPR target sequences
  source 1..23: mol_type = other DNA
cgtggccaccaactggctgcggg

SEQ ID NO: 338 - DNA, 23 bp, synthetic construct
  misc_feature 1..23: note = Cypor CRISPR target sequences
  source 1..23: mol_type = other DNA
caggtcggcctgcagaaacaggg

SEQ ID NO: 339 - DNA, 23 bp, synthetic construct
  misc_feature 1..23: note = Cypor CRISPR target sequences
  source 1..23: mol_type = other DNA
cactttggggtggaagccactgg

SEQ ID NO: 340 - DNA, 23 bp, synthetic construct
  misc_feature 1..23: note = Cypor CRISPR target sequences
  source 1..23: mol_type = other DNA
ttaccaggtcatactcctcaggg

SEQ ID NO: 341 - DNA, 23 bp, synthetic construct
  misc_feature 1..23: note = Cypor CRISPR target sequences
  source 1..23: mol_type = other DNA
ggtgcccacatctacgtctgtgg

SEQ ID NO: 342 - DNA, 23 bp, synthetic construct
  misc_feature 1..23: note = Cypor CRISPR target sequences
  source 1..23: mol_type = other DNA
agacagacgtggatctctctggg

SEQ ID NO: 343 - DNA, 23 bp, synthetic construct
  misc_feature 1..23: note = Cypor CRISPR target sequences
  source 1..23: mol_type = other DNA
ctcctctgcagtccccgtctggg

SEQ ID NO: 344 - DNA, 23 bp, synthetic construct
  misc_feature 1..23: note = Cypor CRISPR target sequences
  source 1..23: mol_type = other DNA
gaacggattcttggcatcaaagg

SEQ ID NO: 345 - DNA, 23 bp, synthetic construct
  misc_feature 1..23: note = Cypor CRISPR target sequences
  source 1..23: mol_type = other DNA
atcaagaaactgatgaccaaggg

SEQ ID NO: 346 - DNA, 23 bp, synthetic construct
  misc_feature 1..23: note = Cypor CRISPR target sequences
  source 1..23: mol_type = other DNA
ggtggtcgatggggggccgcagg

SEQ ID NO: 347 - DNA, 23 bp, synthetic construct
  misc_feature 1..23: note = Cypor CRISPR target sequences
  source 1..23: mol_type = other DNA
aacagtttcatgatcaacatggg

SEQ ID NO: 348 - DNA, 23 bp, synthetic construct
  misc_feature 1..23: note = Cypor CRISPR target sequences
  source 1..23: mol_type = other DNA
ccaggtgagcaagtgcccgcagg

SEQ ID NO: 349 - DNA, 23 bp, synthetic construct
  misc_feature 1..23: note = Cypor CRISPR target sequences
  source 1..23: mol_type = other DNA
tggggtggaagccactggcgagg

SEQ ID NO: 350 - DNA, 23 bp, synthetic construct
  misc_feature 1..23: note = Cypor CRISPR target sequences
  source 1..23: mol_type = other DNA
ggtgggcgtccttggacaggcgg

SEQ ID NO: 351 - DNA, 23 bp, synthetic construct
  misc_feature 1..23: note = Cypor CRISPR target sequences
  source 1..23: mol_type = other DNA
ggttgtggagtacgagaccaagg

SEQ ID NO: 352 - DNA, 23 bp, synthetic construct
  misc_feature 1..23: note = Cypor CRISPR target sequences
  source 1..23: mol_type = other DNA
tgttccgtgcatccctgggcagg

SEQ ID NO: 353 - DNA, 23 bp, synthetic construct
  misc_feature 1..23: note = Cypor CRISPR target sequences
  source 1..23: mol_type = other DNA
ggtcctgtccctgtttctgcagg

SEQ ID NO: 354 - DNA, 23 bp, synthetic construct
  misc_feature 1..23: note = Cypor CRISPR target sequences
  source 1..23: mol_type = other DNA
caaggtgtacatgggggagatgg

SEQ ID NO: 355 - DNA, 23 bp, synthetic construct
  misc_feature 1..23: note = Cypor CRISPR target sequences
  source 1..23: mol_type = other DNA
gcgcatctttgagctggggttgg

SEQ ID NO: 356 - DNA, 23 bp, synthetic construct
  misc_feature 1..23: note = Cypor CRISPR target sequences
  source 1..23: mol_type = other DNA
cctgtgtctgccttccttagggg

SEQ ID NO: 357 - DNA, 23 bp, synthetic construct
  misc_feature 1..23: note = Cypor CRISPR target sequences
  source 1..23: mol_type = other DNA
ggacgtagacctggggccaaggg

SEQ ID NO: 358 - DNA, 23 bp, synthetic construct
  misc_feature 1..23: note = Cypor CRISPR target sequences
  source 1..23: mol_type = other DNA
acctccttgcctgcgtggcaggg

SEQ ID NO: 359 - DNA, 23 bp, synthetic construct
  misc_feature 1..23: note = Cypor CRISPR target sequences
  source 1..23: mol_type = other DNA
cttgaagggcaggcggaactggg

SEQ ID NO: 360 - DNA, 23 bp, synthetic construct
  misc_feature 1..23: note = Cypor CRISPR target sequences
  source 1..23: mol_type = other DNA
gtggtccccagattcatacctgg

SEQ ID NO: 361 - DNA, 23 bp, synthetic construct
  misc_feature 1..23: note = Cypor CRISPR target sequences
  source 1..23: mol_type = other DNA
acccttcataggcttcatccagg

SEQ ID NO: 362 - DNA, 23 bp, synthetic construct
  misc_feature 1..23: note = Cypor CRISPR target sequences
  source 1..23: mol_type = other DNA
cgatctctggcaggctgctcagg

SEQ ID NO: 363 - DNA, 23 bp, synthetic construct
  misc_feature 1..23: note = Cypor CRISPR target sequences
  source 1..23: mol_type = other DNA
cacggccctcacctactacctgg

SEQ ID NO: 364 - DNA, 23 bp, synthetic construct
  misc_feature 1..23: note = Cypor CRISPR target sequences
  source 1..23: mol_type = other DNA
accctgtgtctgccttccttagg

SEQ ID NO: 365 - DNA, 23 bp, synthetic construct
  misc_feature 1..23: note = Cypor CRISPR target sequences
  source 1..23: mol_type = other DNA
ggccaccacgcctgtcatcatgg

SEQ ID NO: 366 - DNA, 23 bp, synthetic construct
  misc_feature 1..23: note = Cypor CRISPR target sequences
  source 1..23: mol_type = other DNA
cgatggggggccgcagggacggg

SEQ ID NO: 367 - DNA, 23 bp, synthetic construct
  misc_feature 1..23: note = Cypor CRISPR target sequences
  source 1..23: mol_type = other DNA
ggggcgcaccttgccctcgccgg

SEQ ID NO: 368 - DNA, 23 bp, synthetic construct
  misc_feature 1..23: note = Cypor CRISPR target sequences
  source 1..23: mol_type = other DNA
tcacacaggtggtcgatgggggg

SEQ ID NO: 369 - DNA, 23 bp, synthetic construct
  misc_feature 1..23: note = Cypor CRISPR target sequences
  source 1..23: mol_type = other DNA
aacggattcttggcatcaaaggg

SEQ ID NO: 370 - DNA, 23 bp, synthetic construct
  misc_feature 1..23: note = Cypor CRISPR target sequences
  source 1..23: mol_type = other DNA
cggccgccgttctccccggcagg

SEQ ID NO: 371 - DNA, 23 bp, synthetic construct
  misc_feature 1..23: note = Cypor CRISPR target sequences
  source 1..23: mol_type = other DNA
taacagtttcatgatcaacatgg

SEQ ID NO: 372 - DNA, 23 bp, synthetic construct
  misc_feature 1..23: note = Cypor CRISPR target sequences
  source 1..23: mol_type = other DNA
gaaccagaagccgtgagtggagg

SEQ ID NO: 373 - DNA, 23 bp, synthetic construct
  misc_feature 1..23: note = Cypor CRISPR target sequences
  source 1..23: mol_type = other DNA
tcctcggcccagcttggaggagg

SEQ ID NO: 374 - DNA, 23 bp, synthetic construct
  misc_feature 1..23: note = Cypor CRISPR target sequences
  source 1..23: mol_type = other DNA
atgctggaaaggggagaccaagg

SEQ ID NO: 375 - DNA, 23 bp, synthetic construct
  misc_feature 1..23: note = Cypor CRISPR target sequences
  source 1..23: mol_type = other DNA
gatgtccaggtagtaggtgaggg

SEQ ID NO: 376 - DNA, 23 bp, synthetic construct
  misc_feature 1..23: note = Cypor CRISPR target sequences
  source 1..23: mol_type = other DNA
ctggcgcagttccacagggacgg

SEQ ID NO: 377 - DNA, 23 bp, synthetic construct
  misc_feature 1..23: note = Cypor CRISPR target sequences
  source 1..23: mol_type = other DNA
gaagtcctgggcattgtcggtgg

SEQ ID NO: 378 - DNA, 23 bp, synthetic construct
  misc_feature 1..23: note = Cypor CRISPR target sequences
  source 1..23: mol_type = other DNA
caccttggaggatgaggcgatgg

SEQ ID NO: 379 - DNA, 23 bp, synthetic construct
  misc_feature 1..23: note = Cypor CRISPR target sequences
  source 1..23: mol_type = other DNA
aggcatgtcagcggaccctgagg

SEQ ID NO: 380 - DNA, 23 bp, synthetic construct
  misc_feature 1..23: note = Cypor CRISPR target sequences
  source 1..23: mol_type = other DNA
catcaagaaactgatgaccaagg

SEQ ID NO: 381 - DNA, 23 bp, synthetic construct
  misc_feature 1..23: note = Cypor CRISPR target sequences
  source 1..23: mol_type = other DNA
acaagggcgtggccaccaactgg

SEQ ID NO: 382 - DNA, 23 bp, synthetic construct
  misc_feature 1..23: note = Cypor CRISPR target sequences
  source 1..23: mol_type = other DNA
ggagctgtacctgagctgggtgg

SEQ ID NO: 383 - DNA, 23 bp, synthetic construct
  misc_feature 1..23: note = Cypor CRISPR target sequences
  source 1..23: mol_type = other DNA
acagagttggggtggacctgtgg

SEQ ID NO: 384 - DNA, 23 bp, synthetic construct
  misc_feature 1..23: note = Cypor CRISPR target sequences
  source 1..23: mol_type = other DNA
cagctcacacaggtggtcgatgg

SEQ ID NO: 385 - DNA, 23 bp, synthetic construct
  misc_feature 1..23: note = Cypor CRISPR target sequences
  source 1..23: mol_type = other DNA
tgagctgggtggtggaggcccgg

SEQ ID NO: 386 - DNA, 23 bp, synthetic construct
  misc_feature 1..23: note = Cypor CRISPR target sequences
  source 1..23: mol_type = other DNA
gcaggtgctggacgtagacctgg

SEQ ID NO: 387 - DNA, 23 bp, synthetic construct
  misc_feature 1..23: note = Cypor CRISPR target sequences
  source 1..23: mol_type = other DNA
aagtcctgggcattgtcggtggg

SEQ ID NO: 388 - DNA, 23 bp, synthetic construct
  misc_feature 1..23: note = Cypor CRISPR target sequences
  source 1..23: mol_type = other DNA
ggaactgcgccagctcctcccgg

SEQ ID NO: 389 - DNA, 23 bp, synthetic construct
  misc_feature 1..23: note = Cypor CRISPR target sequences
  source 1..23: mol_type = other DNA
gcttcatccaggagcgggcctgg

SEQ ID NO: 390 - DNA, 23 bp, synthetic construct
  misc_feature 1..23: note = Cypor CRISPR target sequences
  source 1..23: mol_type = other DNA
cacccaggattttgcccagctgg

SEQ ID NO: 391 - DNA, 23 bp, synthetic construct
  misc_feature 1..23: note = Cypor CRISPR target sequences
  source 1..23: mol_type = other DNA
gacaggcgtggtggccttgaagg

SEQ ID NO: 392 - DNA, 23 bp, synthetic construct
  misc_feature 1..23: note = Cypor CRISPR target sequences
  source 1..23: mol_type = other DNA
gcgggccaaggagcctgccgggg

SEQ ID NO: 393 - DNA, 23 bp, synthetic construct
  misc_feature 1..23: note = Cypor CRISPR target sequences
  source 1..23: mol_type = other DNA
cgtggctgagctcggggccatgg

SEQ ID NO: 394 - DNA, 23 bp, synthetic construct
  misc_feature 1..23: note = Cypor CRISPR target sequences
  source 1..23: mol_type = other DNA
ttgcctgcgtggcaggggccggg

SEQ ID NO: 395 - DNA, 23 bp, synthetic construct
  misc_feature 1..23: note = Cypor CRISPR target sequences
  source 1..23: mol_type = other DNA
ctacctgtaccgggaggagctgg

SEQ ID NO: 396 - DNA, 23 bp, synthetic construct
  misc_feature 1..23: note = Cypor CRISPR target sequences
  source 1..23: mol_type = other DNA
tcacctggcgagagcagttctgg

SEQ ID NO: 397 - DNA, 23 bp, synthetic construct
  misc_feature 1..23: note = Cypor CRISPR target sequences
  source 1..23: mol_type = other DNA
tggcagctggtacctgattttgg

SEQ ID NO: 398 - DNA, 23 bp, synthetic construct
  misc_feature 1..23: note = Cypor CRISPR target sequences
  source 1..23: mol_type = other DNA
cgtagacctggggccaagggtgg

SEQ ID NO: 399 - DNA, 23 bp, synthetic construct
  misc_feature 1..23: note = Cypor CRISPR target sequences
  source 1..23: mol_type = other DNA
gtccccagattcatacctggagg

SEQ ID NO: 400 - DNA, 23 bp, synthetic construct
  misc_feature 1..23: note = Cypor CRISPR target sequences
  source 1..23: mol_type = other DNA
ggatgcacggaacatggccaggg

SEQ ID NO: 401 - DNA, 23 bp, synthetic construct
  misc_feature 1..23: note = Cypor CRISPR target sequences
  source 1..23: mol_type = other DNA
gggatgcacggaacatggccagg

SEQ ID NO: 402 - DNA, 23 bp, synthetic construct
  misc_feature 1..23: note = Cypor CRISPR target sequences
  source 1..23: mol_type = other DNA
ggaccctgaggagtatgacctgg

SEQ ID NO: 403 - DNA, 23 bp, synthetic construct
  misc_feature 1..23: note = Cypor CRISPR target sequences
  source 1..23: mol_type = other DNA
ggagaaggccacgttgagctggg

SEQ ID NO: 404 - DNA, 23 bp, synthetic construct
  misc_feature 1..23: note = Cypor CRISPR target sequences
  source 1..23: mol_type = other DNA
catacctggagggagatgtgagg

SEQ ID NO: 405 - DNA, 23 bp, synthetic construct
  misc_feature 1..23: note = Cypor CRISPR target sequences
  source 1..23: mol_type = other DNA
gctcacacaggtggtcgatgggg

SEQ ID NO: 406 - DNA, 23 bp, synthetic construct
  misc_feature 1..23: note = Cypor CRISPR target sequences
  source 1..23: mol_type = other DNA
ggggtctccctcaccgtaggtgg

SEQ ID NO: 407 - DNA, 23 bp, synthetic construct
  misc_feature 1..23: note = Cypor CRISPR target sequences
  source 1..23: mol_type = other DNA
gcacatcctggccatcctgcagg

SEQ ID NO: 408 - DNA, 23 bp, synthetic construct
  misc_feature 1..23: note = Cypor CRISPR target sequences
  source 1..23: mol_type = other DNA
ttcttgttggactcctctgcagg

SEQ ID NO: 409 - DNA, 23 bp, synthetic construct
  misc_feature 1..23: note = Cypor CRISPR target sequences
  source 1..23: mol_type = other DNA
gcccagcgcatctttgagctggg

SEQ ID NO: 410 - DNA, 23 bp, synthetic construct
  misc_feature 1..23: note = Cypor CRISPR target sequences
  source 1..23: mol_type = other DNA
aaggtgtacatgggggagatggg

SEQ ID NO: 411 - DNA, 23 bp, synthetic construct
  misc_feature 1..23: note = Cypor CRISPR target sequences
  source 1..23: mol_type = other DNA
cttgccctcgccggaggaggagg

SEQ ID NO: 412 - DNA, 23 bp, synthetic construct
  misc_feature 1..23: note = Cypor CRISPR target sequences
  source 1..23: mol_type = other DNA
caccttgccctcgccggaggagg

SEQ ID NO: 413 - DNA, 23 bp, synthetic construct
  misc_feature 1..23: note = Cypor CRISPR target sequences
  source 1..23: mol_type = other DNA
ggcgcggcagcagctcacacagg

SEQ ID NO: 414 - DNA, 23 bp, synthetic construct
  misc_feature 1..23: note = Cypor CRISPR target sequences
  source 1..23: mol_type = other DNA
cccctgccacgcaggcaaggagg

SEQ ID NO: 415 - DNA, 23 bp, synthetic construct
  misc_feature 1..23: note = Cypor CRISPR target sequences
  source 1..23: mol_type = other DNA
gcgcagcagctcctgctccgagg

SEQ ID NO: 416 - DNA, 23 bp, synthetic construct
  misc_feature 1..23: note = Cypor CRISPR target sequences
  source 1..23: mol_type = other DNA
ctcctcctccggcgagggcaagg

SEQ ID NO: 417 - DNA, 23 bp, synthetic construct
  misc_feature 1..23: note = Cypor CRISPR target sequences
  source 1..23: mol_type = other DNA
cgggcctggctgcgacagcaggg

SEQ ID NO: 418 - DNA, 23 bp, synthetic construct
  misc_feature 1..23: note = Cypor CRISPR target sequences
  source 1..23: mol_type = other DNA
gtcgcagccaggcccgctcctgg

SEQ ID NO: 419 - DNA, 23 bp, synthetic construct
  misc_feature 1..23: note = Cypor CRISPR target sequences
  source 1..23: mol_type = other DNA
aagggtgccaccccggtgccggg

SEQ ID NO: 420 - DNA, 23 bp, synthetic construct
  misc_feature 1..23: note = Cypor CRISPR target sequences
  source 1..23: mol_type = other DNA
gcccaccatgatgacaggcgtgg

SEQ ID NO: 421 - DNA, 23 bp, synthetic construct
  misc_feature 1..23: note = Cypor CRISPR target sequences
  source 1..23: mol_type = other DNA
agggtgagtggggtcccatgggg

SEQ ID NO: 422 - DNA, 23 bp, synthetic construct
  misc_feature 1..23: note = Cypor CRISPR target sequences
  source 1..23: mol_type = other DNA
tggcatcaaaggggctgggtggg

SEQ ID NO: 423 - DNA, 23 bp, synthetic construct
  misc_feature 1..23: note = Cypor CRISPR target sequences
  source 1..23: mol_type = other DNA
tgtcagagagagcagctttgtgg

SEQ ID NO: 424 - DNA, 23 bp, synthetic construct
  misc_feature 1..23: note = Cypor CRISPR target sequences
  source 1..23: mol_type = other DNA
tctccctccaggtatgaatctgg

SEQ ID NO: 425 - DNA, 23 bp, synthetic construct
  misc_feature 1..23: note = Cypor CRISPR target sequences
  source 1..23: mol_type = other DNA
tgtggaaaagatgaagaaaacgg

SEQ ID NO: 426 - DNA, 23 bp, synthetic construct
  misc_feature 1..23: note = Cypor CRISPR target sequences
  source 1..23: mol_type = other DNA
cttggcccgcagccagttggtgg

SEQ ID NO: 427 - DNA, 23 bp, synthetic construct
  misc_feature 1..23: note = Cypor CRISPR target sequences
  source 1..23: mol_type = other DNA
tggacgtagacctggggccaagg

SEQ ID NO: 428 - DNA, 23 bp, synthetic construct
  misc_feature 1..23: note = Cypor CRISPR target sequences
  source 1..23: mol_type = other DNA
caccacgcctgtcatcatggtgg

SEQ ID NO: 429 - DNA, 23 bp, synthetic construct
  misc_feature 1..23: note = Cypor CRISPR target sequences
  source 1..23: mol_type = other DNA
atgaagtcctcctccaagctggg

SEQ ID NO: 430 - DNA, 23 bp, synthetic construct
  misc_feature 1..23: note = Cypor CRISPR target sequences
  source 1..23: mol_type = other DNA
caaccagctgggcaaaatcctgg

SEQ ID NO: 431 - DNA, 23 bp, synthetic construct
  misc_feature 1..23: note = Cypor CRISPR target sequences
  source 1..23: mol_type = other DNA
gaaggtgttctgcacatccctgg

SEQ ID NO: 432 - DNA, 23 bp, synthetic construct
  misc_feature 1..23: note = Cypor CRISPR target sequences
  source 1..23: mol_type = other DNA
aaccagaagccgtgagtggaggg

SEQ ID NO: 433 - DNA, 23 bp, synthetic construct
  misc_feature 1..23: note = Cypor CRISPR target sequences
  source 1..23: mol_type = other DNA
aagccactggcgaggagtccagg

SEQ ID NO: 434 - DNA, 23 bp, synthetic construct
  misc_feature 1..23: note = Cypor CRISPR target sequences
  source 1..23: mol_type = other DNA
aaagctgctctctctgacagagg

SEQ ID NO: 435 - DNA, 23 bp, synthetic construct
  misc_feature 1..23: note = Cypor CRISPR target sequences
  source 1..23: mol_type = other DNA
attcttggcatcaaaggggctgg

SEQ ID NO: 436 - DNA, 23 bp, synthetic construct
  misc_feature 1..23: note = Cypor CRISPR target sequences
  source 1..23: mol_type = other DNA
ctcaccctgctgtcgcagccagg

SEQ ID NO: 437 - DNA, 23 bp, synthetic construct
  misc_feature 1..23: note = Cypor CRISPR target sequences
  source 1..23: mol_type = other DNA
ccacgcaggcaaggaggtggggg

SEQ ID NO: 438 - DNA, 23 bp, synthetic construct
  misc_feature 1..23: note = Cypor CRISPR target sequences
  source 1..23: mol_type = other DNA
gctgtacctgagctgggtggtgg

SEQ ID NO: 439 - DNA, 23 bp, synthetic construct
  misc_feature 1..23: note = Cypor CRISPR target sequences
  source 1..23: mol_type = other DNA
agccgtgagtggagggagcgtgg

SEQ ID NO: 440 - DNA, 23 bp, synthetic construct
  misc_feature 1..23: note = Cypor CRISPR target sequences
  source 1..23: mol_type = other DNA
gggtgagtgagtggggtcactgg

SEQ ID NO: 441 - DNA, 23 bp, synthetic construct
  misc_feature 1..23: note = Cypor CRISPR target sequences
  source 1..23: mol_type = other DNA
ggacgggcagtcctgcaggatgg

SEQ ID NO: 442 - DNA, 23 bp, synthetic construct
  misc_feature 1..23: note = Cypor CRISPR target sequences
  source 1..23: mol_type = other DNA
ctcctgctccgagggctccgagg

SEQ ID NO: 443 - DNA, 23 bp, synthetic construct
  misc_feature 1..23: note = Cypor CRISPR target sequences
  source 1..23: mol_type = other DNA
cgcctcggacacggtggagctgg

SEQ ID NO: 444 - DNA, 23 bp, synthetic construct
  misc_feature 1..23: note = Cypor CRISPR target sequences
  source 1..23: mol_type = other DNA
tcctcctccaagctgggccgagg

SEQ ID NO: 445 - DNA, 23 bp, synthetic construct
  misc_feature 1..23: note = Cypor CRISPR target sequences
  source 1..23: mol_type = other DNA
ttcttggcatcaaaggggctggg

SEQ ID NO: 446 - DNA, 23 bp, synthetic construct
  misc_feature 1..23: note = Cypor CRISPR target sequences
  source 1..23: mol_type = other DNA
ctctcctcggcccagcttggagg

SEQ ID NO: 447 - DNA, 23 bp, synthetic construct
  misc_feature 1..23: note = Cypor CRISPR target sequences
  source 1..23: mol_type = other DNA
aagctcgtactggcgaatgctgg

SEQ ID NO: 448 - DNA, 23 bp, synthetic construct
  misc_feature 1..23: note = Cypor CRISPR target sequences
  source 1..23: mol_type = other DNA
cgagcctcacatctccctccagg

SEQ ID NO: 449 - DNA, 23 bp, synthetic construct
  misc_feature 1..23: note = Cypor CRISPR target sequences
  source 1..23: mol_type = other DNA
cgccctggtggttttctgcatgg

SEQ ID NO: 450 - DNA, 23 bp, synthetic construct
  misc_feature 1..23: note = Cypor CRISPR target sequences
  source 1..23: mol_type = other DNA
ctccctccactcacggcttctgg

SEQ ID NO: 451 - DNA, 23 bp, synthetic construct
  misc_feature 1..23: note = Cypor CRISPR target sequences
  source 1..23: mol_type = other DNA
ggtgggccccggcaccggggtgg

SEQ ID NO: 452 - DNA, 23 bp, synthetic construct
  misc_feature 1..23: note = Cypor CRISPR target sequences
  source 1..23: mol_type = other DNA
tgatgtccaggtagtaggtgagg

SEQ ID NO: 453 - DNA, 23 bp, synthetic construct
  misc_feature 1..23: note = Cypor CRISPR target sequences
  source 1..23: mol_type = other DNA
tgatgccaagaatccgttcctgg

SEQ ID NO: 454 - DNA, 23 bp, synthetic construct
  misc_feature 1..23: note = Cypor CRISPR target sequences
  source 1..23: mol_type = other DNA
ctggctgcaggagacagacgtgg

SEQ ID NO: 455 - DNA, 23 bp, synthetic construct
  misc_feature 1..23: note = Cypor CRISPR target sequences
  source 1..23: mol_type = other DNA
gggtgagtggggtcccatggggg

SEQ ID NO: 456 - DNA, 23 bp, synthetic construct
  misc_feature 1..23: note = Cypor CRISPR target sequences
  source 1..23: mol_type = other DNA
gtgcatccctgggcaggaagagg

SEQ ID NO: 457 - DNA, 23 bp, synthetic construct
  misc_feature 1..23: note = Cypor CRISPR target sequences
  source 1..23: mol_type = other DNA
gtacctgagctgggtggtggagg

SEQ ID NO: 458 - DNA, 23 bp, synthetic construct
  misc_feature 1..23: note = Cypor CRISPR target sequences
  source 1..23: mol_type = other DNA
cgcagcagctcctgctccgaggg

SEQ ID NO: 459 - DNA, 23 bp, synthetic construct
  misc_feature 1..23: note = Cypor CRISPR target sequences
  source 1..23: mol_type = other DNA
tacgtctgtgggtgagtgagtgg

SEQ ID NO: 460 - DNA, 23 bp, synthetic construct
  misc_feature 1..23: note = Cypor CRISPR target sequences
  source 1..23: mol_type = other DNA
ctcctctcctcggcccagcttgg

SEQ ID NO: 461 - DNA, 23 bp, synthetic construct
  misc_feature 1..23: note = Cypor CRISPR target sequences
  source 1..23: mol_type = other DNA
tgagctgctgccgcgcctgcagg

SEQ ID NO: 462 - DNA, 23 bp, synthetic construct
  misc_feature 1..23: note = Cypor CRISPR target sequences
  source 1..23: mol_type = other DNA
tcgcctcatcctccaaggtgagg

SEQ ID NO: 463 - DNA, 23 bp, synthetic construct
  misc_feature 1..23: note = Cypor CRISPR target sequences
  source 1..23: mol_type = other DNA
gccacgcaggcaaggaggtgggg

SEQ ID NO: 464 - DNA, 23 bp, synthetic construct
  misc_feature 1..23: note = Cypor CRISPR target sequences
  source 1..23: mol_type = other DNA
tcttgttggactcctctgcaggg

SEQ ID NO: 465 - DNA, 23 bp, synthetic construct
  misc_feature 1..23: note = Cypor CRISPR target sequences
  source 1..23: mol_type = other DNA
ccctgtgtctgccttccttaggg

SEQ ID NO: 466 - DNA, 23 bp, synthetic construct
  misc_feature 1..23: note = Cypor CRISPR target sequences
  source 1..23: mol_type = other DNA
atgttgatcatgaaactgttagg

SEQ ID NO: 467 - DNA, 23 bp, synthetic construct
  misc_feature 1..23: note = Cypor CRISPR target sequences
  source 1..23: mol_type = other DNA
ctccaagctgggccgaggagagg

SEQ ID NO: 468 - DNA, 23 bp, synthetic construct
  misc_feature 1..23: note = Cypor CRISPR target sequences
  source 1..23: mol_type = other DNA
ttggggtggacctgtggggaggg

SEQ ID NO: 469 - DNA, 23 bp, synthetic construct
  misc_feature 1..23: note = Cypor CRISPR target sequences
  source 1..23: mol_type = other DNA
ggccctcaccttggaggatgagg

SEQ ID NO: 470 - DNA, 23 bp, synthetic construct
  misc_feature 1..23: note = Cypor CRISPR target sequences
  source 1..23: mol_type = other DNA
gcagggtgagtggggtcccatgg

SEQ ID NO: 471 - DNA, 23 bp, synthetic construct
  misc_feature 1..23: note = Cypor CRISPR target sequences
  source 1..23: mol_type = other DNA
ggcagtcctgcaggatggccagg

SEQ ID NO: 472 - DNA, 23 bp, synthetic construct
  misc_feature 1..23: note = Cypor CRISPR target sequences
  source 1..23: mol_type = other DNA
ggatggtgagtgccacagtcagg

SEQ ID NO: 473 - DNA, 23 bp, synthetic construct
  misc_feature 1..23: note = Cypor CRISPR target sequences
  source 1..23: mol_type = other DNA
gttggggtggacctgtggggagg

SEQ ID NO: 474 - DNA, 23 bp, synthetic construct
  misc_feature 1..23: note = Cypor CRISPR target sequences
  source 1..23: mol_type = other DNA
tgctggaaaggggagaccaaggg

SEQ ID NO: 475 - DNA, 23 bp, synthetic construct
  misc_feature 1..23: note = Cypor CRISPR target sequences
  source 1..23: mol_type = other DNA
tggccatgcagaaaaccaccagg

SEQ ID NO: 476 - DNA, 23 bp, synthetic construct
  misc_feature 1..23: note = Cypor CRISPR target sequences
  source 1..23: mol_type = other DNA
ggcatcaaaggggctgggtgggg

SEQ ID NO: 477 - DNA, 23 bp, synthetic construct
  misc_feature 1..23: note = Cypor CRISPR target sequences
  source 1..23: mol_type = other DNA
cagggtgagtggggtcccatggg

SEQ ID NO: 478 - DNA, 23 bp, synthetic construct
  misc_feature 1..23: note = Cypor CRISPR target sequences
  source 1..23: mol_type = other DNA
gcacagatgtgcacagagttggg

SEQ ID NO: 479 - DNA, 23 bp, synthetic construct
  misc_feature 1..23: note = Cypor CRISPR target sequences
  source 1..23: mol_type = other DNA
gcaggagctgctgcgcaagatgg

SEQ ID NO: 480 - DNA, 23 bp, synthetic construct
  misc_feature 1..23: note = Cypor CRISPR target sequences
  source 1..23: mol_type = other DNA
gctcacacggccctccccacagg

SEQ ID NO: 481 - DNA, 23 bp, synthetic construct
  misc_feature 1..23: note = Cypor CRISPR target sequences
  source 1..23: mol_type = other DNA
gcggcagcagctcacacaggtgg

SEQ ID NO: 482 - DNA, 23 bp, synthetic construct
  misc_feature 1..23: note = Cypor CRISPR target sequences
  source 1..23: mol_type = other DNA
gatggtgagtgccacagtcaggg

SEQ ID NO: 483 - DNA, 23 bp, synthetic construct
  misc_feature 1..23: note = Cypor CRISPR target sequences
  source 1..23: mol_type = other DNA
agatgtgcacagagttggggtgg

SEQ ID NO: 484 - DNA, 23 bp, synthetic construct
  misc_feature 1..23: note = Cypor CRISPR target sequences
  source 1..23: mol_type = other DNA
gcatccctgggcaggaagagggg

SEQ ID NO: 485 - DNA, 23 bp, synthetic construct
  misc_feature 1..23: note = Cypor CRISPR target sequences
  source 1..23: mol_type = other DNA
gcagggacgggcagtcctgcagg

SEQ ID NO: 486 - DNA, 23 bp, synthetic construct
  misc_feature 1..23: note = Cypor CRISPR target sequences
  source 1..23: mol_type = other DNA
ggttggtggtgactgcagccagg

SEQ ID NO: 487 - DNA, 23 bp, synthetic construct
  misc_feature 1..23: note = Cypor CRISPR target sequences
  source 1..23: mol_type = other DNA
caccatgatgacaggcgtggtgg

SEQ ID NO: 488 - DNA, 23 bp, synthetic construct
  misc_feature 1..23: note = Cypor CRISPR target sequences
  source 1..23: mol_type = other DNA
tggctgggtacacagccacgtgg

SEQ ID NO: 489 - DNA, 23 bp, synthetic construct
  misc_feature 1..23: note = Cypor CRISPR target sequences
  source 1..23: mol_type = other DNA
tgcctgtctcttccctgcagagg

SEQ ID NO: 490 - DNA, 23 bp, synthetic construct
  misc_feature 1..23: note = Cypor CRISPR target sequences
  source 1..23: mol_type = other DNA
ggaggcccggaggcacatcctgg

SEQ ID NO: 491 - DNA, 23 bp, synthetic construct
  misc_feature 1..23: note = Cypor CRISPR target sequences
  source 1..23: mol_type = other DNA
cgggaatgggtgcttcttgttgg

SEQ ID NO: 492 - DNA, 23 bp, synthetic construct
  misc_feature 1..23: note = Cypor CRISPR target sequences
  source 1..23: mol_type = other DNA
ctcctctgcagggaagagacagg

SEQ ID NO: 493 - DNA, 23 bp, synthetic construct
  misc_feature 1..23: note = Cypor CRISPR target sequences
  source 1..23: mol_type = other DNA
gctgggtggtggaggcccggagg

SEQ ID NO: 494 - DNA, 23 bp, synthetic construct
  misc_feature 1..23: note = Cypor CRISPR target sequences
  source 1..23: mol_type = other DNA
agcaccccctcttcctgcccagg

SEQ ID NO: 495 - DNA, 23 bp, synthetic construct
  misc_feature 1..23: note = Cypor CRISPR target sequences
  source 1..23: mol_type = other DNA
cgtctgtgggtgagtgagtgggg

SEQ ID NO: 496 - DNA, 23 bp, synthetic construct
  misc_feature 1..23: note = Cypor CRISPR target sequences
  source 1..23: mol_type = other DNA
atcaaaggggctgggtggggagg

SEQ ID NO: 497 - DNA, 23 bp, synthetic construct
  misc_feature 1..23: note = Cypor CRISPR target sequences
  source 1..23: mol_type = other DNA
tgcctgcgtggcaggggccgggg

SEQ ID NO: 498 - DNA, 23 bp, synthetic construct
  misc_feature 1..23: note = Cypor CRISPR target sequences
  source 1..23: mol_type = other DNA
cacagatgtgcacagagttgggg

SEQ ID NO: 499 - DNA, 23 bp, synthetic construct
  misc_feature 1..23: note = Cypor CRISPR target sequences
  source 1..23: mol_type = other DNA
acagcaccacccttggccccagg

SEQ ID NO: 500 - DNA, 23 bp, synthetic construct
  misc_feature 1..23: note = Cypor CRISPR target sequences
  source 1..23: mol_type = other DNA
gggcctccaccacccagctcagg

SEQ ID NO: 501 - DNA, 23 bp, synthetic construct
  misc_feature 1..23: note = Cypor CRISPR target sequences
  source 1..23: mol_type = other DNA
cggcccctgccacgcaggcaagg

SEQ ID NO: 502 - DNA, 23 bp, synthetic construct
  misc_feature 1..23: note = Cypor CRISPR target sequences
  source 1..23: mol_type = other DNA
acgtctgtgggtgagtgagtggg

SEQ ID NO: 503 - DNA, 23 bp, synthetic construct
  misc_feature 1..23: note = Cypor CRISPR target sequences
  source 1..23: mol_type = other DNA
cacttacaatgtctgaattttgg

SEQ ID NO: 504 - DNA, 23 bp, synthetic construct
  misc_feature 1..23: note = Cypor CRISPR target sequences
  source 1..23: mol_type = other DNA
gctgctctctctgacagaggagg

SEQ ID NO: 505 - DNA, 23 bp, synthetic construct
  misc_feature 1..23: note = Cypor CRISPR target sequences
  source 1..23: mol_type = other DNA
acaggcgtggtggccttgaaggg

SEQ ID NO: 506 - DNA, 23 bp, synthetic construct
  misc_feature 1..23: note = Cypor CRISPR target sequences
  source 1..23: mol_type = other DNA
gcaccccctcttcctgcccaggg

SEQ ID NO: 507 - DNA, 23 bp, synthetic construct
  misc_feature 1..23: note = Cypor CRISPR target sequences
  source 1..23: mol_type = other DNA
tgcatccctgggcaggaagaggg

SEQ ID NO: 508 - DNA, 23 bp, synthetic construct
  misc_feature 1..23: note = Cypor CRISPR target sequences
  source 1..23: mol_type = other DNA
cctgtgtccacgcaggtgtttgg

SEQ ID NO: 509 - DNA, 23 bp, synthetic construct
  misc_feature 1..23: note = Cypor CRISPR target sequences
  source 1..23: mol_type = other DNA
catccctgggcaggaagaggggg

SEQ ID NO: 510 - DNA, 23 bp, synthetic construct
  misc_feature 1..23: note = Cypor CRISPR target sequences
  source 1..23: mol_type = other DNA
ctgggccgaggagaggagagggg

SEQ ID NO: 511 - DNA, 23 bp, synthetic construct
  misc_feature 1..23: note = Cypor CRISPR target sequences
  source 1..23: mol_type = other DNA
tctgcagggaagagacaggcagg

SEQ ID NO: 512 - DNA, 23 bp, synthetic construct
  misc_feature 1..23: note = Cypor CRISPR target sequences
  source 1..23: mol_type = other DNA
gctgggccgaggagaggagaggg

SEQ ID NO: 513 - DNA, 23 bp, synthetic construct
  misc_feature 1..23: note = Cypor CRISPR target sequences
  source 1..23: mol_type = other DNA
gggagaaggccacgttgagctgg

SEQ ID NO: 514 - DNA, 23 bp, synthetic construct
  misc_feature 1..23: note = Cypor CRISPR target sequences
  source 1..23: mol_type = other DNA
agctgggccgaggagaggagagg

SEQ ID NO: 515 - DNA, 23 bp, synthetic construct
  misc_feature 1..23: note = Cypor CRISPR target sequences
  source 1..23: mol_type = other DNA
cttcttttttctttctgaagagg

SEQ ID NO: 516 - DNA, 23 bp, synthetic construct
  misc_feature 1..23: note = Cypor CRISPR target sequences
  source 1..23: mol_type = other DNA
caccccggcccctgccacgcagg

SEQ ID NO: 517 - RNA, 80 nt, synthetic construct
  misc_feature 1..80: note = gRNA
  source 1..80: mol_type = other RNA
gttttagagctagaaatagcaagttaaaataaggctagtccgttatcaacttgaaaaagtggcaccgagtcggtgctttt

SEQ ID NO: 518 - DNA, 20 bp, synthetic construct
  misc_feature 1..20: note = oligonucleotide
  source 1..20: mol_type = other DNA
tcgtgggggtcctgacctac

SEQ ID NO: 519 - DNA, 25 bp, synthetic construct
  misc_feature 1..25: note = oligonucleotide
  source 1..25: mol_type = other DNA
caccgtcgtgggggtcctgacctac

SEQ ID NO: 520 - DNA, 25 bp, synthetic construct
  misc_feature 1..25: note = oligonucleotide
  source 1..25: mol_type = other DNA
aaacgtaggtcaggacccccacgac

SEQ ID NO: 521 - RNA, 43 nt, synthetic construct
  misc_feature 1..43: note = Hammerhead ribozyme
  variation 1..6: note = n = a, c, g, or u
  source 1..43: mol_type = other RNA
nnnnnnctgatgagtccgtgaggacgaaacgagtaagctcgtc

SEQ ID NO: 522 - RNA, 68 nt, synthetic construct
  misc_feature 1..68: note = HDV ribozyme
  source 1..68: mol_type = other RNA
ggccggcatggtcccagcctcctcgctggcgccggctgggcaacatgcttcggcatggcgaatgggac

SEQ ID NO: 523 - RNA, 100 nt, synthetic construct
  source 1..100: mol_type = other RNA
  misc_feature 1..100: note = Guide RNA
  variation 1..20: note = n = a, c, g, or u
nnnnnnnnnnnnnnnnnnnngttttagagctagaaatagcaagttaaaataaggctagtccgttatcaactttaaaaagtggcaccgagtcggtgctttt

(19) United States
(12) Patent Application Publication
(10) Pub. No.: US 2026/0347855 A1
(43) Pub. Date: Nov. 5, 2026

(54) METHODS OF GENE THERAPY

(71) Applicant: OREGON HEALTH & SCIENCE UNIVERSITY, Portland, OR (US)

(72) Inventors: Markus GROMPE, Portland, OR (US); Amita TIYABOONCHAI, Portland, OR (US)

(21) Appl. No.: 19/546,721

(22) Filed: Feb. 23, 2026

Related U.S. Application Data
(63) Continuation of application No. 17/045,806, filed on Apr. 30, 2019, now Pat. No. 12,584,131.
(60) Provisional application No. 62/664,932, filed on Apr. 30, 2018.
(60) Provisional application No. 62/664,930, filed on Apr. 30, 2018.

(51) Int. Cl. C12N 15/113; A61K 31/167; C12N 9/22; C12N 15/86

(52) U.S. Cl. CPC C12N 15/1137; A61K 31/167; C12N 9/22; C12N 15/86; C12N 2310/14; C12N 2310/531; C12N 2750/14141

(57) ABSTRACT

Compositions and methods for gene and/or cell editing are provided.

This application is a continuation application of U.S. patent application Ser. No. 17/045,806, filed Oct. 7, 2020, which is a § 371 application of PCT/US2019/029890, filed Apr. 30, 2019, which claims priority under 35 U.S.C. § 119(e) to U.S. Provisional Patent Application No. 62/664,930, filed Apr. 30, 2018, and U.S. Provisional Patent Application No. 62/664,932, filed Apr. 30, 2018. The foregoing applications are incorporated by reference herein.

This invention was made with government support under R01 DK048252 and R01 DK123093 awarded by the National Institutes of Health. The government has certain rights in the invention.

Incorporated herein by reference in its entirety is the Sequence Listing being concurrently submitted as a XML file named SeqList, created Aug. 12, 2026, and having a size of 678,414 bytes.

### FIELD OF THE INVENTION

The present invention relates to the field of gene and/or cell therapy. Specifically, compositions and methods for therapeutic gene and/or cell therapy by selecting for successfully modified cells are disclosed.

### BACKGROUND OF THE INVENTION

Several publications and patent documents are cited throughout the specification in order to describe the state of the art to which this invention pertains. Each of these citations is incorporated herein by reference as though set forth in full.

Many genetic and acquired disorders affecting cells expressing cytochrome p450 (CYP) proteins are amenable to gene and/or cell therapy. Conceptually, the correction of disease-causing mutations by gene editing is the most elegant and safe method to achieve gene and/or cell therapy. Currently, recombinant adeno-associated virus (rAAV) vectors are the dominant gene therapy platform. For gene editing applications, rAAV vectors are designed to integrate therapeutic payloads into genomic loci of interest through homologous recombination. However, precise gene editing by homologous recombination in vivo has poor efficiency, typically resulting in sub-therapeutic and impermanent gene editing. Indeed, rAAV vectors remain mostly episomal and are lost with cell division. Moreover, random integration of adeno-associated viruses has been associated with liver cancer in both animals and humans, indicating a risk of harm to not only hepatocytes, but any other tissues.

One current method to address the limitations of the rAAV strategy is to directly repair the mutation(s). However in vivo gene repair with presently available methods also has poor efficiency. Another approach is to integrate a therapeutic transgene lacking its own promoter downstream of a cellular promoter into the chromosomal locus of an expressed gene. However, this method has also proven to have low efficiency.

The low efficiency of these methods can be overcome by selective amplification of cells bearing the desired gene editing event. For example, hepatocytes expressing human factor 9 properly targeted to the albumin locus can be selected by conferring resistance to drug-induced toxicity to 4-[(2-carboxyethyl)-hydroxyphosphinyl]-3-oxobutryrate (CEHPOBA), a small-molecule inhibitor of fumarylacetoacetate hydrolase, by using a short-hairpin RNA (shRNA) embedded in a microRNA within an intron to knock down tyrosine catabolic enzyme 4-OH-phenylpyruvate dioxygenase (HHPD) (Nygaard, et al. (2016) Sci. Transl. Med., 8(342):342ra79. However, use of CEHPOBA and exploitation of the tyrosine catabolic pathway for selection has limited application in vivo.

In view of the foregoing, it is clear that superior methods of gene and/or cell therapy are needed.

### SUMMARY OF THE INVENTION

In accordance with the instant invention, methods of propagating and/or expanding a cell population are provided. Methods of expressing a nucleic acid of interest (e.g., a transgene) in a cell or subject are also provided (e.g., improved cell and/or gene therapy methods). In some embodiments, the method comprises inhibiting (e.g., knocking out or knocking down) one or more CYP enzymes, CTNNB1, and/or CYPOR in a cell and administering a prodrug (protoxin) to the cells. The methods may further comprise administering a nucleic acid of interest (e.g., a transgene) to the cell prior to selection with the prodrug. The administered prodrug (protoxin) is metabolized into a toxin in untreated cells but is not metabolized into a toxin in treated cells, thereby allowing for the propagating and/or expanding a desired cell population and/or expression of a nucleic acid of interest (e.g., a transgene). The steps of the method can be performed in vivo and/or in vitro. In certain embodiments, the step of inhibiting one or more CYP enzymes, CTNNB1, and/or CYPOR can be performed in vitro or in vivo. In certain embodiments, the step of administering a prodrug (protoxin) are performed by administering the prodrug (protoxin) to the subject. The step of inhibiting one or more CYP enzymes, CTNNB1, and/or CYPOR can be performed by administering an inhibitory nucleic acid molecule to the cell and/or utilizing gene editing tools such as CRISPR.

### BRIEF DESCRIPTION OF THE DRAWINGS

FIG. 1 provides a general schematic showing the principle of achieving drug selection by inactivating the metabolism of an inert prodrug (protoxin) into a toxic metabolite. The normal status of the cell is depicted on the left, where the prodrug (protoxin) is metabolized to a toxic metabolite. The knockdown or knockout of CYP enzymes, cytochrome P450 reductase (CYPOR or POR), or beta-catenin (Ctnnb1) protects the cells and allows them to proliferate.

FIG. 2 provides a graph of blood alanine aminotransferase (ALT) levels, an indicator of liver damage, over time in mice injected with pX330-Cypor and treated with acetaminophen.

FIG. 3 provides CYPOR immunohistochemistry images of liver samples from mice transduced with pX330-Cypor that were either untreated (top image) or treated with acetaminophen (bottom image). Dashed line outlines CYPOR null nodules.

FIG. 4 provides a graph of the percentage of indels in mice injected with a control plasmid (pX330), pX330-Cypor without acetaminophen selection, pX330-Cypor with acetaminophen injections, and pX330-Cypor with an acetaminophen diet.

FIG. 5 provides immunohistochemistry images of liver samples from mice injected with a Cyp1A2/2E1 CRISPR knockout plasmid that were either untreated (left image) or treated with acetaminophen (right image). Arrows indicate Cyp2E1 null nodules.

FIG. 6 provides CYPOR immunohistochemistry and fluorescence imaging of liver samples from mice transduced with GFP transposons bearing either a control shRNA (top panels) or a Cypor shRNA (bottom panels) and treated with acetaminophen.

FIG. 7 provides a schematic of a self-cleaving guide RNA. Hammerhead ribozyme is SEQ ID NO: 521. HDV ribozyme is SEQ ID NO: 522. Mature gRNA is SEQ ID NO: 523.

FIG. 8 provides luciferase images of livers of neonatal mice injected with a vector comprising luciferase and a self-cleaving guide RNA against Cypor and injected with cas9 at weaning, followed by acetaminophen injections for 8 weeks. Live luciferase imaging was performed on the same mouse at baseline and after 5, 11, and 16 doses.

FIG. 9 provides a graph of human Factor IX levels in the blood of mice injected with saline or with AAV GRCyporF9 as neonates and given a single dose of Cas9 at weaning. Mice were then either untreated or treated with acetaminophen twice weekly. Human Factor IX levels were measured after 0, 3, 9 and 15 doses of acetaminophen.

FIG. 10 provides a graph of human Factor IX levels in the blood of adult mice injected with AAV GRCyporF9 and treated with acetaminophen. Human Factor IX levels were determined after 0, 2, 7, 13 and 19 doses.

FIG. 11 provides images of CYPOR (top panels) and human Factor IX (bottom panels) immunohistochemistry of tissue samples from GRCyporF9 treated mice after acetaminophen treatment. In top panels, CYPOR negative tissue is indicated by dotted line and arrows. In bottom panels, mouse hepatocytes positive for human Factor IX are indicated by dotted line and arrows.

### DETAILED DESCRIPTION OF THE INVENTION

The obstacles observed with gene and/or cell therapy can be overcome through the use of: (1) methods designed to exploit and/or disrupt any gene loci required for the metabolic activity of CYP enzymes wherein the selective amplification and/or expansion of resistant cell populations through drug-induced toxicity is effected by toxins with broad application in vivo, (2) site-specific gene editing methods designed to link a desired modification to a selectable gene disruption in cis, such that selective amplification and/or cell expansion occurs only if the modification occurs at the specified site, (3) site-specific gene editing methods designed to effect a desired modification and selectable gene disruption that do not require an endonuclease enzyme to cut DNA or the use of promoters to activate gene expression such that selective amplification and/or cell expansion occurs only if the modification occurs at the specified site, (4) site-neutral gene editing methods designed to effect the desired modification and gene disruption using randomly-integrating vectors with their own endogenous promoters such that the selective amplification and/or cell expansion only occurs if integration occurs, irrespective of site, (5) methods that knock-down, knock-out, or otherwise disrupt gene loci required for the metabolic activity of CYP enzymes wherein the selective amplification and/or expansion of desired cell populations occurs without a gene editing event or modification, or (6) any combination of any of (1)-(5). The instant invention provides methods which accomplish the above.

The instant invention involves the utilization of the cytochrome p450 enzyme (CYP) system for achieving high efficiency, cell and/or tissue specific, clinically effective, higher permanence gene editing and/or therapy. The instant invention does not require a pre-existing genetic disadvantage in the cells to be edited. Rather, in some embodiments, the instant invention creates a disadvantage in un-edited cells by conferring an advantageous resistance to drug-induced toxicity to properly edited cells. This advantageous resistance can be conferred unto the cells by knocking down, knocking out, or otherwise disrupting: (1) one or more CYP genes that encode CYP enzymes, (2) the cytochrome p450 reductase (CYPOR or POR) gene, (3) the CTNNB1 (beta-catenin) gene, (4) any gene loci required for metabolic activity of CYP enzymes, or (5) any combination of any of (1)-(4). Moreover, advantageous resistance can also be conferred by disabling, disrupting, or otherwise inactivating one or more CYP enzyme, CTNNB1, and/or CYPOR directly. Upon administration of an appropriate toxin (e.g., a prodrug or protoxin), cells with advantageous resistance (i.e., lack the ability to metabolize and activate the toxin due to reduction in CYP activity) will preferentially propagate over those cells which were not edited.

In accordance with one aspect of the instant invention, methods of propagating and/or expanding a cell population are provided. In some embodiments, the method comprises inhibiting (e.g., knocking out or knocking down) one or more CYP enzymes, CTNNB1, and/or CYPOR in a cell, and administering a prodrug (protoxin) to the cells, wherein the administered prodrug (protoxin) is metabolized into a toxin in untreated cells (e.g., cells wherein CYP enzymes, CTNNB1, and/or CYPOR are not inhibited) but is not metabolized into a toxin in treated cells (e.g., cells wherein CYP enzymes, CTNNB1, and/or CYPOR are inhibited). The steps of the method can be performed in vivo and/or in vitro, including ex vivo methods (e.g., wherein cells from a subject (autologous cells) are treated in vitro and then administered back to the subject. In certain embodiments, the step of inhibiting one or more CYP enzymes, CTNNB1, and/or CYPOR can be performed in vitro or in vivo. In certain embodiments, the step of administering a prodrug (protoxin) can be performed in vitro or in vivo (e.g., the prodrug (protoxin) can be administered generally to the subject comprising the cells or administered directly to the cells).

In accordance with another aspect of the instant invention, methods of expressing a nucleic acid of interest (e.g., a transgene) in a cell are provided. In some embodiments, the method comprises inhibiting (e.g., knocking out or knocking down) one or more CYP enzymes, CTNNB1, and/or CYPOR in a cell, introducing the nucleic acid of interest (e.g., transgene) into the cell, and administering a prodrug (protoxin) to the cells, wherein the administered prodrug (protoxin) is metabolized into a toxin in untreated cells (e.g., cells wherein CYP enzymes, CTNNB1, and/or CYPOR are not inhibited) but is not metabolized into a toxin in treated cells (e.g., cells wherein CYP enzymes, CTNNB1, and/or CYPOR are inhibited). The steps of the method can be performed in vivo and/or in vitro, including ex vivo methods (e.g., wherein cells from a subject (autologous cells) are treated in vitro and then administered back to the subject. In certain embodiments, the step of inhibiting one or more CYP enzymes, CTNNB1, and/or CYPOR and/or introducing the nucleic acid of interest (e.g., transgene) can be performed in vitro or in vivo. The inhibition of the one or more CYP enzymes, CTNNB1, and/or CYPOR and introduction of the nucleic acid of interest (e.g., transgene) may be performed simultaneously or in cis such that both events occur within the same advantaged resistant cells. In certain embodiments, the step of administering a prodrug (protoxin) can be performed in vitro or in vivo (e.g., the prodrug (protoxin) can be administered generally to the subject comprising the cells or administered directly to the cells).

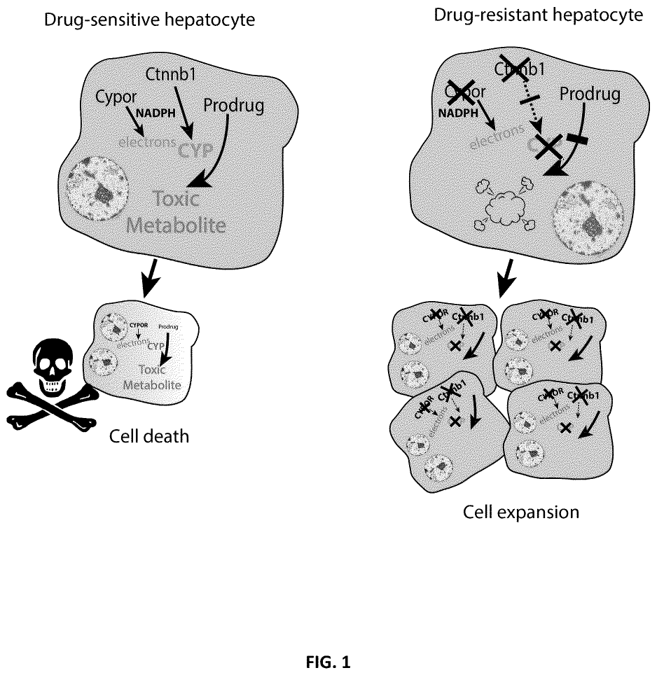

FIG. 1 provides a general schematic showing the principle of achieving drug selection by inactivating the metabolism of an inert prodrug into a toxic metabolite. The normal status of the cell is depicted on the left. The metabolizing enzyme is expressed in normal cells, resulting in toxicity after metabolic conversion. If the gene(s) responsible for the metabolic activity are either knocked down (e.g., siRNA or shRNA) or knocked out (e.g., targeted nuclease (e.g., CRISPR)), the toxic metabolite is not produced after administration of the toxic precursor. Knockdown or knockout cells are protected and can proliferate, whereas enzyme positive cells die. As seen in FIG. 1, the knockdown or knockout of a CYP enzymes, cytochrome P450 reductase (CYPOR or POR), or beta-catenin (Ctnnb1) can prevent the conversion of the prodrug (protoxin) such as acetaminophen to its toxic metabolite. Notably, POR donates electrons from NADPH to all CYP enzymes and, hence, is essential for their activity. In the absence of POR, all CYP enzymes are inactive. Similarly, Ctnnb1 is essential for the transcription of genes expressed in zone 3 hepatocytes, including Cyp1A2, 2E1 and 3A4. At least these CYP enzymes will be absent from Ctnnb1 inhibited cells.

The cells of the methods of the instant invention can be any cell type. In some embodiments, the cells have CYP activity. In some embodiments, the cells are hepatocytes.

The inhibition (e.g., knockout or knockdown) of one or more CYP enzymes, CTNNB1, and/or CYPOR in a cell can be performed by any method known in the art. In a particular embodiment, the inhibition is achieved by administering an inhibitor of one or more CYP enzymes, CTNNB1, and/or CYPOR to the cell. Such inhibitors are compounds which reduce the activity of the target gene and/or protein (e.g., inhibit or reduce substrate interaction) and/or reduce the expression of the target gene and/or protein. Examples of inhibitors include, without limitation, proteins, polypeptides, peptides, antibodies, small molecules, and nucleic acid molecules. Preferably, the inhibitor is a nucleic acid-based inhibitor. In some embodiments, the inhibitor is an inhibitory nucleic acid molecule, such as an antisense, siRNA, or shRNA molecule (or a nucleic acid molecule encoding the inhibitory nucleic acid molecule). In some embodiments, the inhibitor is a gene editing inhibitor such as a CRISPR based targeting of the desired gene (e.g., with a guide RNA targeting the desired gene).

CYP genes are well known in the art and include any cytochrome p450 gene. Examples of CYP genes are provided in Nelson, D. R. (Human Genomics (2009) 4:59-65; incorporated by reference herein). Examples of CYP genes include, without limitation, cytochrome p450 gene families 1 through 51. Examples of CYP genes include, without limitation, CYP1A (e.g., CYP1A1, CYP1A2), CYP1B (e.g., CYP1B1), CYP2A (e.g., CYP2A6, CYP2A7, CYP2A13), CYP2B (e.g., CYP2B6), CYP2C (e.g., CYP2C8, CYP2C9, CYP2C18, CYP2C19), CYP2D (e.g., CYP2D6), CYP2E (e.g., CYP2E1), CYP2F (e.g., CYP2F1), CYP2J (e.g., CYP2J2), CYP2R (e.g., CYP2R1), CYP2S (e.g., CYP2S1), CYP2U (e.g., CYP2U1), CYP2W (e.g., CYP2W1), CYP3A (e.g., CYP3A4, CYP3A5, CYP3A7, CYP3A43), CYP4A (e.g., CYP4A11, CYP4A22), CYP4B (e.g., CYP4B1), CYP4F (e.g., CYP4F2, CYP4F3, CYP4F8, CYP4F11, CYP4F12, CYP4F22), CYP4V (e.g., CYP4V2), CYP4X (e.g., CYP4X1), CYP4Z (e.g., CYP4Z1), CYP5A (e.g., CYP5A1) CYP7A (e.g., CYP7A1), CYP7B (e.g., CYP7B1), CYP8B (e.g., CYP8B1), CYP11A (e.g., CYP11A1), CYP11B (e.g., CYP11B1, CYP111B2), CYP17A (e.g., CYP17A1), CYP 19A (e.g., CYP19A1), CYP20 (e.g., CYP20A1), CYP21A (e.g., CYP21A2), CYP24A (e.g., CYP24A1), CYP26A (e.g., CYP26A1), CYP26B (e.g., CYP26B1), CYP26C (e.g., CYP26C1), CYP27A (e.g., CYP27A1), CYP27B (e.g., CYP27B1), CYP27C (e.g., CYP27C1), CYP39A (e.g., CYP39A1), CYP46A (e.g., CYP46A1), and CYP51A (e.g., CYP51A1). In a particular embodiment, the CYP gene is from a subfamily selected from CYP1, CYP2, or CYP3.

Cytochrome P450 reductase (CYPOR or POR; the terms have been used interchangeably herein) is an endoplasmic reticulum membrane oxidoreductase protein with FAD and FMN binding domains which allows it to donate electrons directly from NADPH to all cytochrome p450 enzymes (lyanagi, et al., (2012) Arch. Biochem. Biophys., 528:72-89). In humans, the cytochrome P450 reductase gene is found at locus: chromosome 7, q-arm, region 1, band 1, sub-band 23 (7q11.23) (see, e.g., GenBank Accession No. NC_000007.14). Examples of nucleotide and amino acids sequences of human POR can be found, for example, at Gen Bank Gene ID: 5447 and GenBank Accession Nos. NM_000941.3 and NP_000932.3.

Catenin beta-1 (CTNNB1; also known as β-catenin) is a dual function protein involved in regulation and coordination of cell-cell adhesion and gene transcription. Examples of nucleotide and amino acids sequence of human Ctnnb1 can be found, for example, at Gen Bank Gene ID: 1499 and GenBank Accession Nos. NM_001098209.1 and NP_001091679.1.

As stated hereinabove, the genome of the cell may be edited to inactivate/inhibit one or more CYP enzymes, CTNNB1, and/or CYPOR. The genome of the cells can be edited using any method known in the art such as, without limitation: zinc finger nucleases (ZFNs), transcription activator like effector nucleases (TALENs), clustered regularly interspaced short palindromic repeats (CRISPRs), and meganucleases. In some embodiments, CRISPR is utilized. Clustered, regularly interspaced, short palindromic repeat (CRISPR)/Cas9 (e.g., from Streptococcus pyogenes) technology and gene editing are well known in the art (see, e.g., Shi et al. (2015) Nat. Biotechnol., 33(6):661-7; Sander et al. (2014) Nature Biotech., 32:347-355; Jinek et al. (2012) Science, 337:816-821; Cong et al. (2013) Science 339:819-823; Ran et al. (2013) Nature Protocols 8:2281-2308; Mali et al. (2013) Science 339:823-826; Sapranauskas et al. (2011) Nucleic Acids Res. 39:9275-9282; Nishimasu et al. (2014) Cell 156(5):935-49; Swarts et al. (2012) PLoS One, 7:e35888; Sternberg et al. (2014) Nature 507(7490):62-7; addgene.org/crispr/guide). The RNA-guided CRISPR/Cas9 system involves expressing Cas9 along with a guide RNA molecule (gRNA). Guidelines and computer-assisted methods for generating gRNAs are available (see, e.g., CRISPR Design Tool (crispr.mit.edu); Hsu et al. (2013) Nat. Biotechnol. 31:827-832; addgene.org/CRISPR; and CRISPR gRNA Design tool—DNA2.0 (dna20.com/eCommerce/startCas9)). When coexpressed, gRNAs bind and recruit Cas9 to a specific genomic target sequence where it mediates a double strand DNA (dsDNA) break. More than one gRNA (e.g., 2) may be administered to make multiple breaks within the target DNA. The double strand break can be repaired by non-homologous end joining (NHEJ) pathway yielding an insertion and/or deletion or, in the presence of a donor template, by homology-directed repair (HDR) pathway for replacement mutations (Overballe-Petersen et al. (2013) Proc. Natl. Acad. Sci., 110:19860-19865; Gong et al. (2005) Nat. Struct. Mol. Biol., 12:304-312). While CRISPR is described herein as utilizing Cas9, other nucleases such as Cas9 variants and homologs can be used. Other examples include, without limitation, Streptococcus pyogenes Cas9, Cas9 D10A, high fidelity Cas9 (Kleinstiver et al. (2016) Nature, 529:490-495; Slaymaker et al. (2016) Science, 351:84-88), Cas9 nickase (Ran et al. (2013) Cell, 154:1380-1389), Streptococcus pyogenes Cas9 with altered PAM specificities (e.g., SpCas9_VQR, SpCas9_EQR, and SpCas9_VRER; Kleinstiver et al. (2015) Nature, 523:481-485), Staphylococcus aureus Cas9, cas12a (Cpf1) (Rusk, N., Nat. Methods (2019) 16(3):215), the CRISPR/Cpf1 system of Acidaminococcus, and the CRISPR/Cpf1 system of Lachnospiraceae.

The binding specificity of the CRISPR/Cas9 complex depends on two different elements. First, the binding complementarity between the targeted genomic DNA (genDNA) sequence and the complementary recognition sequence of the gRNA (e.g., ~18-22 nucleotides, particularly about 20 nucleotides). Second, the presence of a protospacer-adjacent motif (PAM) juxtaposed to the genDNA/gRNA complementary region (Jinek et al. (2012) Science 337:816-821; Hsu et al. (2013) Nat. Biotech., 31:827-832; Sternberg et al. (2014) Nature 507:62-67). The PAM motif for S. Pyogenes Cas9 has been fully characterized, and is NGG or NAG (Jinek et al. (2012) Science 337:816-821; Hsu et al. (2013) Nat. Biotech., 31:827-832). Other PAMs of other Cas9 proteins are also known (see, e.g., addgene.org/crispr/guide/#pam-table). Typically, the PAM sequence is 3′ of the DNA target sequence in the genomic sequence.

The guide RNA may comprise separate nucleic acid molecules. For example, one RNA may specifically hybridize to a target sequence (crRNA) and another RNA (trans-activating crRNA (tracrRNA)) specifically hybridizes with the crRNA. Preferably, the guide RNA is a single molecule (sgRNA) which comprises a sequence which specifically hybridizes with a target sequence (crRNA; complementary sequence) and a sequence recognized by Cas9 (e.g., a tracrRNA sequence; scaffold sequence). Examples of gRNA scaffold sequences are well known in the art (e.g., 5′-GUUUUAGAGC UAGAAAUAGC AAGUUAAAAU AAGGCUAGUC CGUUAUCAAC UUGAAAAAGU GGCACCGAGU CGGUGCUUUU (SEQ ID NO: 517)). As used herein, the term “specifically hybridizes” does not mean that the nucleic acid molecule needs to be 100% complementary to the target sequence. Rather, the sequence may be at least 80%, 85%, 90%, 95%, 97%, 99%, or 100% complementary to the target sequences (e.g., the complementary between the gRNA and the genomic DNA). The greater the complementarity reduces the likelihood of undesired cleavage events at other sites of the genome. In a particular embodiment, the region of complementarity (e.g., between a guide RNA and a target sequence) is at least about 10, at least about 12, at least about 15, at least about 17, at least about 20, at least about 25, at least about 30, at least about 35, or more nucleotides. In a particular embodiment, the region of complementarity (e.g., between a guide RNA and a target sequence) is about 15 to about 25 nucleotides, about 15 to about 23 nucleotides, about 16 to about 23 nucleotides, about 17 to about 21 nucleotides, about 18 to about 22 nucleotides, or about 20 nucleotides. In a particular embodiment, the guide RNA targets a sequence or comprises a sequence (e.g., RNA version) which has at least 80%, 85%, 90%6, 95%, 97%, 99%, or 100% homology or identity to one of SEQ ID NOs: 34 to 516. The sequences may be extended or shortened by 1, 2, 3, 4, or 5 nucleotides at the end of the sequence opposite from the PAM (e.g., at the 5′ end). When the sequence is extended, the added nucleotides should correspond to the genomic sequence.

In some embodiments, the gRNA is a self-cleaving guide RNA (scgRNA). A self-cleaving gRNA comprises ribozymes at the 5′ and 3′ ends of a single guide RNA. Upon expression of a scgRNA, the self-cleaving ribozymes release the gRNA within the cell to act with Cas9. In some embodiments, the scgRNA comprises a hammerhead ribozyme at the 5′ of the gRNA and a hepatitis delta virus ribozyme at the 3′ end of the gRNA. In some embodiments, the scgRNA is under the control of a pol2 promoter. In some embodiments, the scgRNA is under the control of a tissue specific promoter. In some embodiments, the scgRNA is under the control of the albumin promoter.

In some embodiments, the method comprises administering at least one Cas9 (e.g., the protein and/or a nucleic acid molecule encoding Cas9) and at least one gRNA (e.g., a nucleic acid molecule encoding the gRNA) to the cell or subject. In a particular embodiment, the Cas9 is S. pyogenes Cas9. In a particular embodiment, the targeted PAM is in the 5′UTR, 3′UTR, promoter, or intron (e.g., first intron). The nucleic acids of the instant invention may be administered consecutively (before or after) and/or at the same time (concurrently). The nucleic acid molecules may be administered in the same composition or in separate compositions. In a particular embodiment, the nucleic acid molecules are delivered in a single vector (e.g., a viral vector or a plasmid).

As stated hereinabove, an inhibitory nucleic acid molecule, such as an antisense, siRNA, or shRNA molecule (or a nucleic acid molecule encoding the inhibitory nucleic acid molecule), may be used to inactivate/inhibit one or more CYP enzyme, CTNNB1, and/or CYPOR. When an inhibitory nucleic acid molecule (e.g., an shRNA) is delivered to a cell or subject, the inhibitory nucleic acid molecule may be administered directly or an expression vector may be used. Exemplary target sequences for inhibitory nucleic acid molecules (e.g., shRNA) include, without limitation, any one of SEQ ID NOs: 2 to 33. In some embodiments, the shRNA is embedded in a microRNA.

The administration of nucleic acid molecules into cells (including to a subject) can be achieved by any method known in the art. In some embodiments, the nucleic acid molecules are introduced into cells by infection (e.g., when a viral vector is employed). In some embodiment, the nucleic acid molecules are delivered to the cells by injection, transfection, electroporation, biolistic particle delivery system (e.g., gene gun), sonoporation (e.g., cellular sonication to increase cell permeability), magnetofection (e.g., use of magnetic field to bring particles containing nucleic acid into the target cells), hydrodynamic delivery, lipoplex delivery, polymersome delivery, polyplex delivery, dendrimer delivery, nanoparticle delivery (e.g., inorganic nanoparticles), and/or through the use of cell-penetrating peptides.

The nucleic acid molecules of the instant invention may be contained within a vector (e.g., plasmid, transposon, viral vector (e.g., adeno-associated viruses (AAVs), adenoviruses, retroviruses, and lentiviruses), etc.). In some embodiments, the desired nucleic acid sequences may be expressed from appropriate promoters within the vector including a strong promoter, a constitutive promoter, tissue or cell specific promoter (e.g., hepatocyte specific promoter), and/or a regulated promoter. Examples of promoters are well known in the art and include, but are not limited to, RNA polymerase II promoters, the T7 RNA polymerase promoter, and RNA polymerase III promoters (e.g., U6 and H1; see, e.g., Myslinski et al. (2001) Nucl. Acids Res., 29:2502-09). In some embodiments, the desired nucleic acid sequences are expressed from an endogenous promoter (e.g., a promoter within the cell).

In some embodiments, the vector is an integrating vector. In further embodiments, the integrating vector does not require an endonuclease enzyme to cut DNA or the use of promoters to activate gene expression (e.g., utilizes GeneRide™ technology (LogicBio Therapeutics, Cambridge, MA)). GeneRide™ is a gene editing strategy using homologous recombination to enable site-specific transfer of genetic material that does not require an endonuclease enzyme to cut DNA or the use of promoters to activate gene expression. In further embodiments, the methods utilize randomly-integrating vectors with their own endogenous promoters such that integration occurs randomly. In some embodiments, the invention utilizes non-integrating vectors.

In some embodiments, the vector is a viral vector, particularly a rAAV vector. In some embodiments, the vector is a plasmid DNA vector. In some embodiments, the invention utilizes synthetic oligonucleotide vectors. In some embodiments, the invention utilizes retroviral vectors such as vectors based on foamy virus, oncovirus, and/or lentivirus. In another embodiment, the vector is based on adenoviruses. In another embodiment, the vector is based on AAV. In another embodiment, the vector is based on envelope protein pseudotyping of viral vectors. In another embodiment, the vector is based on replication-competent vectors and cis and trans-acting elements. In another embodiment, the vector is based on Herpes Simplex Virus.

In some embodiments, the prodrug (protoxin) administered to the cells is a compound (e.g., a small molecule) which is metabolized by CYP activity or an activity dependent on CYP activity. The toxicity of the prodrug (protoxin) to be administered to the cells can be tested on the cells to determine whether the toxin metabolite is sufficiently toxic such that an advantageous resistance can be achieved in edited cells. In some embodiment, the lethality of the toxin can be measured, wherein a toxin which causes cellular death or apoptosis to untreated cells is sufficiently toxic such that an advantageous resistance can be achieved in edited cells. In some embodiment, biomarkers within the cells can be measured to determine if the toxin is sufficiently toxic. For example, with regard to hepatocytes, a toxin may be determined to be sufficiently toxic if elevated levels of certain liver enzymes are detected including, but not limited to: alanine aminotransferase (ALT), aspartate transaminase (AST), alkaline phosphatase (ALP), or gamma-glutamyl transpeptidase (GGT). Increased expression of one or more of these liver enzymes is known to be indicative of hepatotoxicity, wherein the presence of at least mild to moderate hepatotoxicity evidences the necessary toxicity. Moreover, dosage guidelines to achieve mild to moderate hepatotoxicity are known in the art (see, e.g., Jaeschke, H. (2015) Dig. Dis., 33:464-471; Arafa, et al. (2018) Toxicol. Appl. Pharmacol., 346:37-44; Calvo, et al. (2017) Invest New Drugs, 36(3):476-486; Fashe et al. (2015) Chem. Res. Toxicol., 28:702-710; Huttunen et al. (2008) Curr. Med. Chem., 15:2346-2365; Maruyama et al. (1995) Dig. Dis. Sci., 40:2602-2607; McEneny-King et al. (2017) Bioorg. Med. Chem. Lett., 27:2443-2449).

Hepatotoxicity may be graded using the 5-point scale developed by the Drug-Induced Liver Injury Network (DILIN) (see, e.g., Fontana, et al. (2009) Drug Safety, 32:55-68). Mild hepatoxicity (or 1+) is defined as: raised serum aminotransferase or alkaline phosphatase levels or both, but total serum bilirubin is less than <2.5 mg/dL and coagulopathy is detected (INR<1.5). Moderate hepatoxicity (or 2+) is defined as: raised serum aminotransferase or alkaline phosphatase levels or both and a total serum bilirubin level of greater than or equal to 2.5 mg/dL or coagulopathy (IND ≥1.5) without hyperbilirubinemia. Moderate to severe hepatoxicity (or 3+) is defined as: raised serum aminotransferase or alkaline phosphatase levels and total serum bilirubin level of ≥2.5 mg/dL and hospitalization (or preexisting hospitalization is prolonged) because of the drug induced liver injury. Severe hepatoxicity (or 4+) is defined as: raised serum aminotransferase or alkaline phosphatase levels and serum bilirubin of ≥2.5 mg/dL and at least one of the following: (a) prolonged jaundice and symptoms beyond three (3) months, (b) signs of hepatic decompensation (INR ≥1.5, ascites, encephalopathy), or (c) other organ failure believed to be related to drug induced liver injury.

As explained hereinabove, the prodrug (protoxin) may be administered directly to the subject. Thus, the invention provides for methods of administering an effective dosage of a prodrug (protoxin) to human beings wherein an elevated or abnormal liver function test from the blood showing mild to moderate hepatotoxicity indicates sufficient dosage to amplify selected or edited cells. In some embodiments, dosage of the acetaminophen sufficient to induce selective toxicity in adults is: (1) 6 grams/day acetaminophen, on consecutive days, or (2) greater than 10 grams acetaminophen as a single dose.

As used herein, a CYP dependent toxin refers to a prodrug (protoxin) whose toxicity depends on activation by cytochrome p450 metabolism involving a CYP enzyme, a Cypor (POR) protein or both. Many CYP dependent toxins are metabolically activated by cytochrome p450 metabolism (see generally Jaeschke, H. (2015) Dig. Dis., 33:464-471; Huttunen et al. (2008) Curr. Med. Chem., 15:2346-2365). Often the parental compound is inert and non-toxic (e.g., a prodrug), but its metabolite(s) generated by Cyp mediated enzymatic conversion are toxic, especially hepatotoxic. For example, acetaminophen is metabolized to the hepatotoxic compound N-acetyl-p-benzoquinone imine (NAPQI) by three POR dependent CYP enzymes (CYP1A2, CYP2E1, and CYP3A4). In some embodiments, Cyp dependent toxins are used for in vivo selection of gene modified hepatocytes or other modified cells, in which the Cyp activity responsible for producing the toxic metabolite has been inactivated. Examples of CYP dependent toxins are provided in Table 1.

TABLE 1
CYP dependent toxins

| Drug | Metabolizing CYP |
|---|---|
| Acetaminophen | CYP2E1, CYP1A2, CYP3A4, CYP2D6 |
| Retrorsine | CYP3A4 |
| Cyclophosphamide | CYP2B6, CYP2C9, CYP3A4 |
| Tamoxifen | CYP3A4 |
| Ketoconazole | CYP3A4 |
| Tramadol | CYP2D6 |
| Tacrine | CYP1A2 |
| Lasiocarpine | CYP3A4 |
| Senkirkin | CYP3A4 |
| Dascarbazine | CYP1A1, CYP1A2, CYP2E1 |
| Tegafur | CYP2A6, CYP1A2, CYP2C8 |

(see, e.g., Jaeschke, H. (2015) Dig. Dis., 33:464-471; Arafa, et al. (2018) Toxicol. Appl. Pharmacol., 346:37-44; Calvo, et al. (2017) Invest New Drugs, 36(3):476-486; Fashe et al. (2015) Chem. Res. Toxicol., 28:702-710; Huttunen et al. (2008) Curr. Med. Chem., 15:2346-2365; Maruyama et al. (1995) Dig. Dis. Sci., 40:2602-2607; McEneny-King et al. (2017) Bioorg. Med. Chem. Lett., 27:2443-2449; Utkarsh, et al. (2016) Chem. Biol. Interact., 255:12-22).

In one aspect, the CYP dependent toxin may include acetaminophen (APAP) as the prodrug that is converted to the active drug by any of: CYP2E1, CYP1A2, CYPA4, or CYP2D6. In another aspect, the CYP dependent toxin may include retrosine as the prodrug that is converted to the active drug by CYP3A4. In another aspect, the CYP dependent toxin may include tangretin. In another aspect, the CYP dependent toxin may include cyclophosphamide as the prodrug that is converted to the active drug phosphoramide mustard by hydroxylation facilitated by any of: CYP2B6, CYP2C9, or CYP3A4. In another aspect, the CYP dependent toxin may include ifosfamide as the prodrug that is converted to the active drug ifosfamide mustard by hydroxylation. In another aspect, the CYP dependent toxin may include trofosfamide as the prodrug that is converted to the active drug trofosfamide mustard by hydroxylation. In another aspect, the CYP dependent toxin may include pradefovir as the prodrug that is converted to the active drug PMEA-triphosphate by hydroxylation. In another aspect, the CYP dependent toxin may include MB07133 as the prodrug that is converted to the active drug araC-triphosphate by hydroxylation. In another aspect, the CYP dependent toxin may include MB07811 as the prodrug that is converted to the active drug MB07344 by hydroxylation. In another aspect, the CYP dependent toxin may include buparvaquone hydroxyimine as the prodrug that is converted to the active drug buparvaquone mustard by oxidation. In another aspect, the CYP dependent toxin may include nabumetone hydroxyimine as the prodrug that is converted to the active drug nabumetone by oxidation. In another aspect, the CYP dependent toxin may include DB289 as the prodrug that is converted to the active drug furamidine (DB75) mustard by 0-demethylation reduction. In another aspect, the CYP dependent toxin may include sibrafiban as the prodrug that is converted to the active drug Ro 48-3888 mustard by reduction. In another aspect, the CYP dependent toxin may include ximelagatran as the prodrug that is converted to the active drug melagartan by reduction. In another aspect, the CYP dependent toxin may include guanoxabenz as the prodrug that is converted to the active drug guanabenz by reduction. In another aspect, the CYP dependent toxin may include AQ4N as the prodrug that is converted to the active drug AQ4 by reduction. In another aspect, the CYP dependent toxin may include dacarbazine (“DTIC”) as the prodrug that is converted to the active drug MTIC by hydroxylation. In another aspect, the CYP dependent toxin may include tegafur as the prodrug that is converted to the active drug 5-FU by hydroxylation. In another aspect, the CYP Dependent Toxin may include 4-ipomeanol as the prodrug that is converted to the active drug by oxidation or epoxidation. In another aspect, the CYP dependent toxin may include DDMX (“PNU-152243”) as the prodrug that is converted to the active drug PNU-159682 by cyclization. In another aspect, the CYP dependent toxin may include tamoxifen as the prodrug that is converted to the active drug 4-hydroxy-tamoxifen by hydroxylation facilitated by CYP3A4. In another aspect, the CYP dependent toxin may include tamoxifen as the prodrug that is converted to the active drug N-Desmethyl-tamoxifen by N-demethylation. In another aspect, the CYP dependent toxin may include tamoxifen as the prodrug that is converted to the active drug endoxifen by hydroxylation or N-demethylation. In another aspect, the CYP dependent toxin may include ketoconazole as a prodrug that is converted to an active drug by CYP3A4. In another aspect, the CYP dependent toxin may include tramadol as a prodrug that is converted to an active drug by CYP2D6. In another aspect, the CYP dependent toxin may include tacrine as a prodrug that is converted to an active drug by CYP1A2. In another aspect, the CYP dependent toxin may include Lasiocarpine as a prodrug that is converted to an active drug by CYP3A4. In another aspect, the CYP dependent toxin may include Senkirkin as a prodrug that is converted to an active drug by CYP3A4. In another aspect, the CYP dependent toxin may include dascarbazine as a prodrug that is converted to an active drug by any of: CYP1A1, CYP1A2, or CYP2C8. In another aspect, the CYP dependent toxin may include tegafur as a prodrug that is converted to an active drug by any of: CYP2A6, CYP1A2, or CYP2C8. In another aspect, the CYP dependent toxin may include clopidogrel as the prodrug that is converted to the active drug R-130964) by oxidation. In another aspect, the CYP dependent toxin may include V-PYRRO/NO as the prodrug that is converted to the active drug PYRRO/NO by oxidation or epoxidation. In another aspect, the CYP dependent toxin may include V-PROLI/NO as the prodrug that is converted to the active drug PROLI/NO by oxidation or epoxidation.

In some embodiments, the methods of the instant invention provide a method for selectively amplifying and/or expanding a population of cells comprising the steps of:
a) administering a donor molecule comprising:
   i) a gRNA (e.g., a scgRNA), wherein the gRNA sequence combines with a cas9 nuclease, resulting in an active, site-nuclease designed to cut, knock down, knock out, and/or otherwise disrupt a CYP enzyme(s), the Cypor gene, or the Ctnnb1 gene;
   ii) a polymerase II promoter (e.g., a cell and/or tissue specific promoter);
   iii) a transgene (optional);
   iv) a cell and/or tissue specific Homology Arm sequence designed to facilitate, promote, or otherwise enable homologous integration into a desired recipient cell locus;
b) administering a cas9 nuclease (e.g., protein or nucleic acid encoding Cas9) wherein the cas9 nuclease combines with a gRNA resulting in an active, site-nuclease designed to cut, knock down, knock out, and/or otherwise disrupt the a CYP enzyme(s), the Cypor gene, or the Ctnnb1 gene; and
c) administering a prodrug (protoxin) (e.g., a CYP dependent toxin).

A scgRNA donor molecule allows expression of functional gRNA (capable of binding to cas9). The expression of the scgRNA may be limited to specific cells and/or tissue types (e.g., those that have undergone a desired gene-editing event). In a particular embodiment, cell-type specific promoters used by mammalian cells use RNA Polymerase II (Pol2) promoters. It is also known in the art that transcription driven by these promoters does not allow for proper processing of cas9 gRNA. It is also known in the art that only recipient cells that have undergone homologous recombination of a DNA donor molecule with an embedded Pol2 promoter sequence will transcribe the DNA donor molecular sequence, including the gRNA and any transgenes the DNA donor molecule may or may not contain. Conversely, it is known in the art that random integration of said homologous DNA donor molecule will not yield functional gsRNA or transgene expression. By embedding a gRNA sequence into a DNA donor molecule with Homology Arms embedded both upstream and downstream from the gRNA sequence, cell and/or tissue specific homologous recombination and integration of the DNA donor molecule into the recipient cell genome are facilitated. Further, by embedding a gRNA sequence into a DNA donor molecule wherein one or more ribozyme sequences are embedded upstream from the gRNA sequence (“left ribozyme”) and one or more ribozyme sequences are embedded downstream from the gRNA sequence (“right ribozyme”), an unspliced Pol2 RNA transcript containing gRNA sequence is properly processed by the combined cleaving by the left ribozyme and the right ribozyme. Thus, the non-random, simultaneous co-expression of functional gRNA and transgenes targeted to specific cell and/or tissue types is made possible by a scgRNA molecule.

In some embodiments of the invention, the gRNA sequence is embedded into an intron of the transgene. In some embodiments, a rAAV vector is utilized. In other embodiments, a pX330 vector is utilized. In some embodiments, the homologous integration is facilitated using GeneRide™ technology wherein both transgene and selection cassette lack their own promoter; after homologous recombination into the target locus both the transgene and the CYP toxin selection cassette are expressed by a cellular promoter; only targeted integrations are selected. In further embodiments, the integration is facilitated using a randomly-integrating vector. In some embodiments, the homologous integration is facilitated using randomly-integrating vectors wherein both transgene and selection cassette have their own promoter(s); after random integration into the cellular chromosome, both the transgene and the CYP Toxin selection cassette are expressed; any chromosomal integration can be selected. In some embodiments, the gRNA sequence is designed to combined with a cas9 nuclease resulting in an active, site-specific nuclease that cuts, knocks down, knocks out, and/or otherwise disrupts individual CYP enzymes. In some embodiments, the gRNA sequence is designed to combined with a cas9 nuclease resulting in an active, site-specific nuclease that cuts, knocks down, knocks out, and/or otherwise disrupts the transcription factor Ctnnb1 (beta-catenin) that is required for the expression of CYP enzymes.

In one aspect, the disclosure provides a scgRNA donor molecule sequence (and all conservatively modified variants thereof) encoding: (a) corresponding inverted terminal repeats (“ITRs”) to aid in concatemer formation set out in residues 1 to 45, and 4,406 to 4,550 of SEQ ID NO: 1, (b) corresponding mouse albumin Homology Arms as set out in residues 212 to 1,516 and 2,978 to 3,012 of SEQ ID NO: 1, (c) a RNA Polymerase II promoter sequence as set out in residues 1,517 to 1582 of SEQ IN NO: 1, (d) an example of a human Factor IX sequence as set out in residues 1,589 to 2,971 of SEQ ID NO: 1, (e) an example of a hammerhead ribozyme sequence as set out in residues 3,026 to 3068 of SEQ ID NO: 1, (f) an example of a gRNA wherein the gRNA comprises a DNA targeting segment complementary to the human POR gene as set out in residues 3,067 to 3,088 of SEQ ID NO: 1, and (g) an example of a hepatitis delta virus ribozyme as set out in residues 3,089 to 3,236 of SEQ ID NO: 1. In some embodiments, the Cypor Human Factor 9 GeneRide™ is included in a plasmid as a rAAV vector. In one embodiment, elements (e) and (g) cleave the unspliced Pol2 promoter generated RNA transcript thereby releasing the properly processed gRNA. In a non-limiting example, the 3′-terminus of element (e)'s sequence is optimally located immediately upstream (5′) from the 5′-terminus of element (f), with no spacing, and the 5′-terminus of element (g) is optimally located immediately downstream (3′) from the 3′-terminus of element (f), with no spacing. In some embodiments, elements (e) and (g) are not located immediately upstream and downstream respectively from element (f) but, may be located as far as 100 base pairs or more (e.g., at least 50, at least 70, or at least 100 base pairs) upstream in the case of element (e), and may be located as far as 100 base pairs or more (e.g., at least 50, at least 70, or at least 100 base pairs) downstream in the case of element (g), from element (f) respectively. In some embodiments, element (f) comprises a sequence complementary to the human POR gene as set out in one of SEQ ID NOs: 34-516.

In some embodiments, the invention provides a method for selectively amplifying and/or expanding a population of cells comprising the steps of:
a) administering a donor molecule comprising:
   i) a polymerase III promoter (e.g., a U6 promoter sequence);
   ii) a gRNA wherein the gRNA sequence combines with a cas9 nuclease, resulting in an active, site-nuclease designed to cut, knock down, knock out, and/or otherwise disrupt a CYP enzyme(s) or the Cypor Gene;
   iii) promoter;
   iv) SpCAS9 sequence wherein the cas9 nuclease expressed will combine with a gsRNA of element (b) resulting in an active, site-nuclease designed to cut, knock down, knock out, and/or otherwise disrupt CYP enzyme(s), the Cypor gene or Ctnnb1; and
b) administering a prodrug (protoxin) (e.g., a CYP dependent toxin).
   In one aspect, the gRNA of element ii) comprises complementary to the human POR gene as set out in one of SEQ ID NOs: 34-516. In some embodiments, a pX330 plasmid vector is utilized. In some embodiments, homologous integration is facilitated using GeneRide™ technology wherein both transgene and selection cassette lack their own promoter; after homologous recombination into the target locus both the transgene and the CYP toxin selection cassette are expressed by a cellular promoter; only targeted integrations are selected. In further embodiments, the homologous integration is facilitated using randomly-integrating vectors wherein both transgene and selection have their own promoter(s); after random integration into the cellular chromosome, both the transgene and the Cypor selection cassette are expressed; any chromosomal integration can be selected.

In some embodiments, the instant invention provides methods for selectively amplifying and/or expanding a population of cells comprising the steps of:
a) administering a donor molecule comprising:
   i) a polymerase III promoter (e.g., a U6 promoter sequence);
   ii) a lentiviral-construct, shRNAmir backbone wherein the shRNA embedded in the microRNA comprises a sequence homologous to the Cypor gene; and
b) administering a prodrug (protoxin) (e.g., a CYP dependent toxin).
   In some aspects, the shRNA of element ii) comprises sequence complementary to the human POR gene as set out in one of SEQ ID NOs: 2-33. In some embodiments, a rAAV vector is utilized. In other embodiments, a pX330 vector is utilized. In some embodiments, the homologous integration is facilitated using GeneRide™ technology wherein both transgene and selection cassette lack their own promoter; after homologous recombination into the target locus both the transgene and the CYP Toxin selection cassette are expressed by a cellular promoter; only targeted integrations are selected. In further embodiments, the integration is facilitated using a randomly-integrating vector. In some embodiments, the homologous integration is facilitated using randomly-integrating vectors wherein both transgene and selection cassette have their own promoter(s); after random integration into the cellular chromosome, both the transgene and the CYP toxin selection cassette are expressed; any chromosomal integration can be selected.

In some embodiments, the method comprises administering an mRNA-based donor molecule to the cells. For example, the RNA molecule may comprise a gRNA and an mRNA encoding Cas9.

In accordance with another aspect of the instant invention, methods of treating, inhibiting, and/or preventing a disease or disorder in a subject are provided. In a particular embodiment, the method comprises performing the methods of the instant invention wherein the transgene is therapeutic (e.g., encodes a therapeutic protein) for the disease or disorder to be treated. In a particular embodiment, the method comprises administering the prodrug (protoxin) to the subject. This method can be used in any system where gene-edited cells can grow in vivo. Examples of target tissues include, without limitation: the liver (e.g., hepatocytes and/or bile ducts), hematopoietic system (e.g., stem cells, T-cells, and/or progenitors), skin (e.g., dermal and/or hair-follicle stem cells), kidney (e.g., tubular epithelium), intestinal tract (e.g., stem cells), lung, and pancreas.

In some embodiments, the disease or disorder to be treated is liver disease. Examples of liver disease include, without limitation, cirrhosis, fibrosis, hepatocellular carcinoma (HCC), and hepatic infection. In some embodiments, the disease or disorder to be treated is selected from the group of genetic liver disorders including Crigler-Najjar syndrome (types 1 and 2), familial hypercholesterolemia, maple syrup urine disease, progressive familial intrahepatic cholestasis, phenylketonuria, tyrosinemia, mucopolysaccharidosis VII, alpha-1 antitrypsin (AAT) deficiency, ornithine transcarbamylase (OTC) deficiency, Wilson's disease, glycogen storage diseases, von Gierke's disease, Pompe's disease, hyperbilirubinemia, acute intermittent porphyria (AIP), and citrullinemia type 1. In some embodiments, the disease or disorder to be treated is selected from the group of hemophilia A, hemophilia B, and oxalosis. In some embodiments, the disease or disorder to be treated is hepatitis B or hepatitis C. In some embodiments, the disease or disorder to be treated is selected from the group of hepatomas, cholangiocarcinomas, metastatic tumors of the liver, and extrahepatic tumors. In some embodiments, the disease or disorder to be treated is selected from the group of acute liver failure, allograft rejection, and xenograft rejection.

The components as described herein will generally be administered to a subject as a pharmaceutical preparation. The term “patient” or “subject” as used herein refers to human or animal subjects. The components of the instant invention may be employed therapeutically, under the guidance of a physician for the treatment of the indicated disease or disorder.

The pharmaceutical preparation comprising the components of the invention may be conveniently formulated for administration with an acceptable medium (e.g., pharmaceutically acceptable carrier) such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

Selection of a suitable pharmaceutical preparation depends upon the method of administration chosen. For example, the components of the invention may be administered by direct injection into any desired tissue (e.g., liver) or into the surrounding area. In this instance, a pharmaceutical preparation comprises the components dispersed in a medium that is compatible with blood or the target tissue.

The therapy may be, for example, administered parenterally, by injection into the blood stream (e.g., intravenous), orally, or by subcutaneous, intramuscular or intraperitoneal injection. In a particular embodiment, the therapy is administered by direct injection (e.g., into the tissue to be treated) or orally. Pharmaceutical preparations for injection are known in the art. If injection is selected as a method for administering the therapy, steps must be taken to ensure that sufficient amounts of the molecules reach their target cells to exert a biological effect.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral or parenteral. In the preparation of an oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Injectable suspensions may be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

The methods of the instant invention may further comprise monitoring the disease or disorder in the subject after administration of the composition(s) of the instant invention to monitor the efficacy of the method.

In accordance with another aspect of the instant invention, methods of expressing a gRNA are provided. The methods comprise expressing a self-cleaving guide RNA (scgRNA) under the control of a pol2 promoter (e.g., a tissue or cell specific pol2 promoter). A self-cleaving gRNA comprises ribozymes at the 5′ and 3′ ends of a single guide RNA. Upon expression of a scgRNA, the self-cleaving ribozymes release the gRNA within the cell to act with Cas9. In some embodiments, the scgRNA comprises a hammerhead ribozyme at the 5′ of the gRNA and a hepatitis delta virus ribozyme at the 3′ end of the gRNA. The expression of scgRNA can used with any gene editing event (e.g., homologous recombination). In a particular embodiment, the scgRNA is used with the gene editing described herein (e.g., to inhibit one or more CYP enzymes, CTNNB1, and/or CYPOR in a cell). In a particular embodiment, the scgRNA is used with the gene editing described in Example 3 (e.g., to inhibit phenylpyruvate dioxygenase (Hpd) or homogentisic acid dioxygenase (Hgd), particularly in a fumarylacetoacetate hydrolase (Fah^{-/-}) background). Vectors comprising an scgRNA under the control of a pol2 promoter (e.g., a tissue or cell specific pol2 promoter) are also encompassed by the instant invention.

### DEFINITIONS

The following definitions are provided to facilitate an understanding of the present invention:
The singular forms “a,” “an,” and “the” include plural referents unless the context clearly dictates otherwise. Any reference to “or” herein is intended to encompass “and/or” unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween® 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in Remington: The Science and Practice of Pharmacy, (Lippincott, Williams and Wilkins); Liberman, et al., Eds., Pharmaceutical Dosage Forms, Marcel Decker, New York, N.Y.; and Rowe, et al., Eds., Handbook of Pharmaceutical Excipients, Pharmaceutical Pr.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof.

As used herein, the term “subject” refers to an animal, particularly a mammal, particularly a human.

The term “isolated” refers to the separation of a compound from other components present during its production. “Isolated” is not meant to exclude artificial or synthetic mixtures with other compounds or materials, or the presence of impurities that do not substantially interfere with the fundamental activity, and that may be present, for example, due to incomplete purification, or the addition of stabilizers.

The term “oligonucleotide,” as used herein, includes a nucleic acid molecule comprised of two or more ribo- and/or deoxyribonucleotides, preferably more than three. The exact size of the oligonucleotide will depend on various factors and on the particular application and use of the oligonucleotide.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

The term “vector” refers to a carrier nucleic acid molecule (e.g., RNA or DNA) into which a nucleic acid sequence can be inserted, e.g., for introduction into a host cell where it may be expressed and/or replicated. A vector may be either RNA or DNA and may be single or double stranded. A vector may be used as a vehicle for delivering exogenous material into a recipient cell via transformation, transfection, transduction or any other mode cell penetrance. An “expression vector” is a specialized vector that contains a gene or nucleic acid sequence with the necessary operably linked regulatory regions needed for expression in a host cell (e.g., promoters, enhancers, translational start signals, polyadenylation signals, terminators, and the like). The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, amino acids, or nucleic acids.

The phrase “small, interfering RNA (siRNA)” refers to a short (typically less than 30 nucleotides long, particularly 12-30 or 20-25 nucleotides in length) double stranded RNA molecule. Typically, the siRNA modulates the expression of a gene to which the siRNA is targeted. Methods of identifying and synthesizing siRNA molecules are known in the art (see, e.g., Ausubel et al., Current Protocols in Molecular Biology, John Wiley and Sons, Inc). Short hairpin RNA molecules (shRNA) typically consist of short complementary sequences (e.g., an siRNA) separated by a small loop sequence (e.g., 6-15 nucleotides, particularly 7-10 nucleotides) wherein one of the sequences is complimentary to the gene target. shRNA molecules are typically processed into an siRNA within the cell by endonucleases. Exemplary modifications to siRNA molecules are provided in U.S. Application Publication No. 20050032733. For example, siRNA and shRNA molecules may be modified with nuclease resistant modifications (e.g., phosphorothioates, locked nucleic acids (LNA), 2′-O-methyl modifications, or morpholino linkages). Expression vectors for the expression of siRNA or shRNA molecules may employ a strong promoter which may be constitutive or regulated.

“Antisense nucleic acid molecules” or “antisense oligonucleotides” include nucleic acid molecules (e.g., single stranded molecules) which are targeted (complementary) to a chosen sequence (e.g., to translation initiation sites and/or splice sites) to inhibit the expression of a protein of interest. Such antisense molecules are typically between about 15 and about 50 nucleotides in length, more particularly between about 15 and about 30 nucleotides, and often span the translational start site of mRNA molecules. Antisense constructs may also be generated which contain the entire sequence of the target nucleic acid molecule in reverse orientation. Antisense oligonucleotides targeted to any known nucleotide sequence can be prepared by oligonucleotide synthesis according to standard methods. Antisense oligonucleotides may be modified as described above to comprise nuclease resistant modifications.

As used herein, “downstream” means a genetic locus or sequence position that is located closer the 3′-terminus in relation to the reference locus, wherein the oligonucleotide sequence begins with the 5′-terminus and runs “5′ to 3′” until ending at the 3′-terminus.

As used herein, “donor molecule” means an exogenous molecule wherein said molecule is designed to cause an effect on a recipient cell.

As used herein “embedded” means the inclusion of certain desired oligonucleotide sequence within a larger synthetic oligonucleotide, forming a single molecule, wherein said molecule may or may not have additional activity or purpose beyond that of the embedded sequence.

As used herein, “homology arms” means target sequence with homology to a desired gene locus designed to promote, facilitate, and enable homologous integration of a donor molecule into a recipient cell genome.

As used herein, “integrating vector” means a vector that delivers donor molecules, transgenes, or other exogenous DNA or RNA, which are integrated into a recipient cell chromosome or genome.

As used herein, “knockout” means a technique by which an organism's gene is made inoperative.

As used herein, “knockdown” means a technique by which the expression of an organism's gene is reduced.

As used herein, “prodrug” means any compound that when administered to a biological system generates the drug substance, i.e., active ingredient, as a result of spontaneous chemical reaction(s), enzyme catalyzed chemical reaction(s), photolysis, and/or metabolic chemical reaction(s). A prodrug is, thus, a covalently modified analog or latent form of a compound with either therapeutic or toxic activity (e.g., a protoxin).

“Linker” refers to a chemical moiety comprising a chain of atoms that covalently attach at least two compounds. The linker can be linked to any synthetically feasible position of the compounds, but preferably in such a manner as to avoid blocking the compounds desired activity. Linkers are generally known in the art. In a particular embodiment, the linker may contain from 1 to about 50 atoms, from 1 to about 10 atoms, or from about 1 to about 5 atoms.

As used herein, “shRNAmir” means a microRNA embedded shRNA molecule.

As used herein, “non-integrating vector” means a vector that delivers donor molecules, transgenes, or other exogenous DNA or RNA wherein said donor molecules remain episomal and/or are not integrated into a recipient cell chromosome or genome.

As used herein, “synthetic oligonucleotide” means an oligonucleotide made by man, and not isolated from an existing natural source.

As used herein, “target sequence” means a DNA polynucleotide that comprises a “target site” or “target sequence.” The terms “target site,” or “target sequence,” are used interchangeably herein to refer to a nucleic acid sequence present in a target DNA to which a DNA-targeting segment of a vector will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Sambrook, et al., Molecular Cloning: A Laboratory Manual, Cold Spring Harbor Laboratory Press). The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “noncomplementary strand” or “non-complementary strand.” By “site-directed modifying polypeptide” or “RNA-binding site-directed polypeptide” or “RNA-binding site-directed modifying polypeptide” or “site-directed polypeptide” it is meant a polypeptide that binds RNA and is targeted to a specific DNA sequence. A site-directed modifying polypeptide as described herein is targeted to a specific DNA sequence by the RNA molecule to which it is bound. The RNA molecule comprises a sequence that binds, hybridizes to, or is complementary to a target sequence within the target DNA, thus targeting the bound polypeptide to a specific location within the target DNA (the target sequence).

As used herein, “transgene” means non-native genetic material that, upon introduction to a recipient cell, has the potential to change the recipient cell's phenotype.

As used herein, “upstream” or means a genetic locus or sequence position that is located closer the 5′-terminus in relation to the reference locus, wherein the oligonucleotide sequence begins with the 5′-terminus and runs “5′ to 3′” until ending at the 3′-terminus.

The following examples are provided to illustrate various embodiments of the present invention. The examples are illustrative and are not intended to limit the invention in any way.

### Example 1

### Materials and Methods

Plasmid construction: A CRISPR recognition site was identified within exon 1 of the mouse Cypor gene using an online design tool (available at www.crispr.mit.edu) having the sequence 5′-TCGTGGGGGTCCTGACCTAC-3′ (SEQ ID NO: 518). Oligonucleotides encoding this sequence were annealed and ligated into pX330 (Addgene cat. 42230; Watertown, MA) cut with BbsI. pX330 is a human codon-optimized SpCas9 and chimeric guide RNA expression plasmid (Cong, et al. Science (2013) 339(6121):819-23). Briefly, the plasmid comprises the gRNA under control of the U6 promoter and SpCas9 under the control of the CBh promoter (a hybrid chicken beta actin promoter). The oligonucleotide sequences were 5′-CACCGTCGTGGGGGTCCTGACCTAC-3′ (SEQ ID NO: 519) and 5′-AAACGTAGGTCAGGACCCCCACGAC-3′ (SEQ ID NO: 520). The resulting plasmid (pX330-Cypor) was validated to cut the targeted sequence using the Guide-It™ Mutation Detection kit (Takara Bio USA, Inc., Mountain View, CA).

Animal procedures: Male 129/sv mice, 7-9 weeks of age, were administered 40 μg of the pX330-Cypor plasmid diluted in saline by hydrodynamic tail vein injection. After three weeks, the mice were divided into two cohorts: four mice were given twice weekly injections of acetaminophen (APAP, 13 mg/ml in saline) by IP injection, 16 injections total. Three mice were reserved as controls and did not receive APAP. For the final 6 APAP doses, the mice were fasted 16 hours prior to injection. Alanine transaminase (ALT) levels were measured 6-20 hours following APAP injection from 10 μl of blood drawn from a saphenous vein puncture using the ALT Reagent Kit (available at BKKits.com, BQ 004A-CR). Mice were sacrificed two weeks after the final APAP injection and the livers were harvested.

Immunofluorescence: Liver sections were fixed in paraformaldehyde for 5 hours, cryoprotected in 30% sucrose overnight, and embedded in optimal cutting temperature (OCT) compound. Seven-micro sections were washed in PBS, permeabilized 12 minutes in phosphate-buffered saline (PBS) with 0.1% Triton X-100, blocked with 0.3 M glycine for 30 minutes, and incubated with primary antibody overnight at 4 °C (Abcam ab180597 (Cambridge, United Kingdom); 1:200 in PBS with 10% normal goat serum). After washing 3×5 minutes in PBS, sections were incubated in secondary antibody (Goat anti-Rabbit Alexa Fluor®555, Invitrogen cat. A27039 (Carlsbad, CA), diluted 1/2000) for 1 hour at room temperature. Sections were mounted in DAPI Fluoromount-G® (Southern Biotech, Inc., Birmingham, AL) and visualized using a Zeiss confocal microscope.

### Results

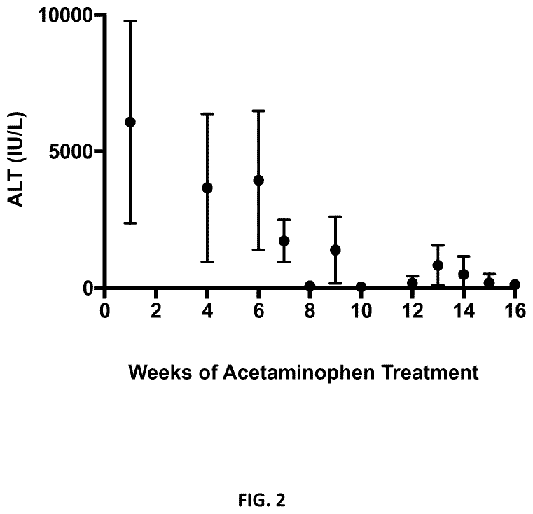

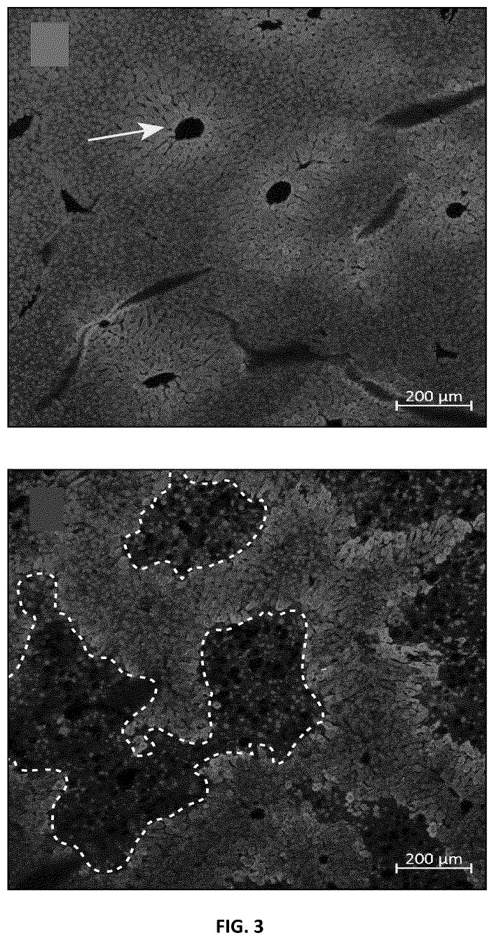

As seen in FIG. 2, liver damage as measured by blood ALT levels decreased over time for the mice injected with pX330-Cypor. The treated mice were APAP resistant after ~10 weeks. CYPOR immunohistochemistry results are provided in FIG. 3. In pX330-Cypor treated mice that received APAP injections, distribution with large CYPOR-negative nodules are present, thereby indicating clonal expansion of Cypor-null hepatocytes (bottom image; dashed line outlines Cypor null nodules). The large CYPOR-negative nodules are not present in mice not treated with APAP (top image).

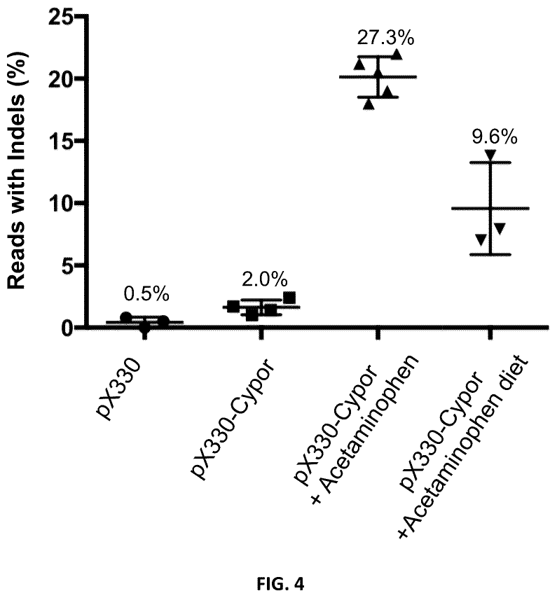

FIG. 4 shows the number of Cypor indels in mice injected with (from left to right) a control plasmid (pX330), pX330-Cypor without APAP selection, pX330-Cypor with APAP injections, and pX330-Cypor with an APAP diet. Indel frequency in APAP treated mice was significantly higher (p<0.01) in mice receiving APAP, either by injection or diet, thereby demonstrating selection of hepatocytes with the Cypor knockout.

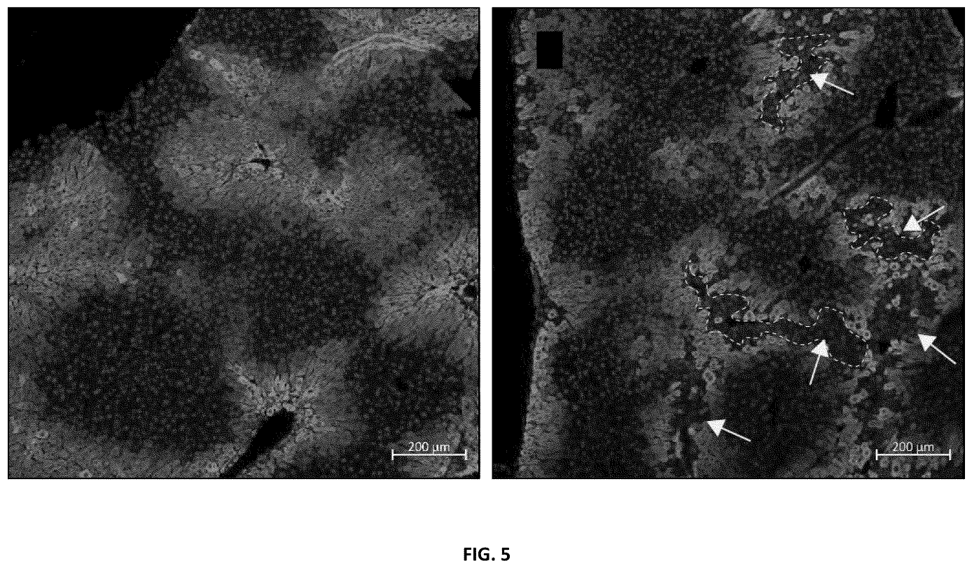

In addition to the above, mice were also injected with a Cyp1A2/2E1 CRISPR knockout plasmid and treated with APAP. As seen in FIG. 5, APAP selected liver of mice injected with the Cyp1A2/2E1 CRISPR knockout plasmid resulted in Cyp2E1 negative nodules (right image; dashed line outlines Cyp2E1 null nodules) compared to mice without APAP treatment (left image). With APAP administration the frequency of Cyp1A2 and Cyp2E1 indels increased from 1 to ~12% (~17% of hepatocytes). This demonstrates that knockout of Cyp1A2 and Cyp2E1 is sufficient to achieve APAP resistance, even in Cypor proficient livers.

### Example 2

The Sleeping Beauty transposon system was used to test CYPOR knockdown by shRNA. Briefly, mice were injected with two separate plasmids. One plasmid contained sleeping beauty transposase under the control of the CMV promoter. The second plasmid contained transposon cassette comprising a GFP expression cassette and U6 driven Cypor shRNA. After treatment, injected mice were exposed to 3 months of APAP selection.

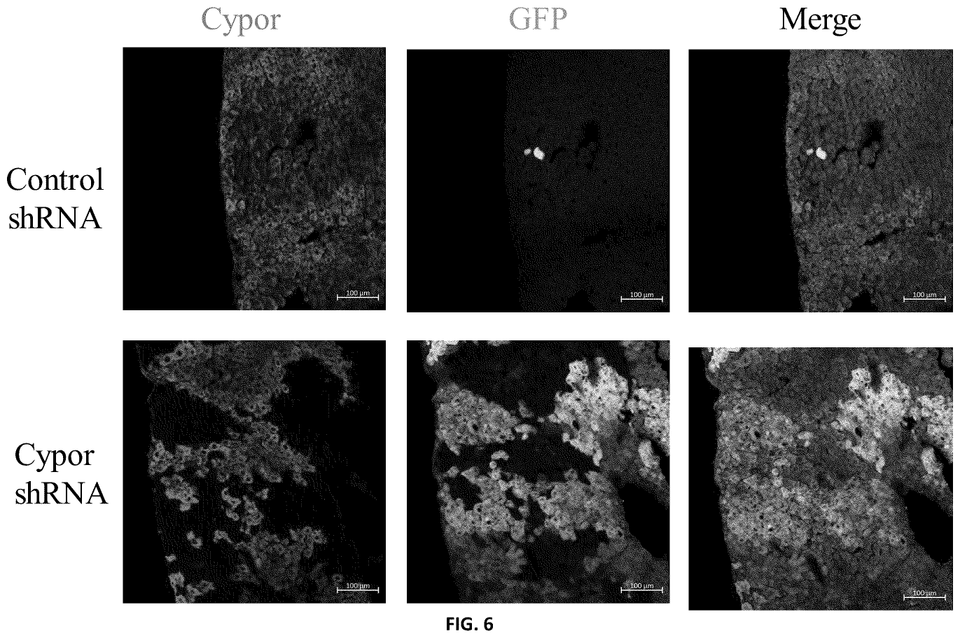

FIG. 6 provides CYPOR immunohistochemistry and GFP fluorescence on livers from mice in the experiments treated with the Sleeping Beauty transposon system and APAP. The top panels show results from a negative control (irrelevant shRNA). Rare GFP+ cells can be seen, but no nodules have formed. The bottom panels show images from a APAP treated mice that were injected with the GFP-Cypor shRNA transposon. Large confluent areas of GFP expression are seen in Cypor negative regions. This provides clear evidence for the expansion of transposon positive hepatocytes.

### Example 3

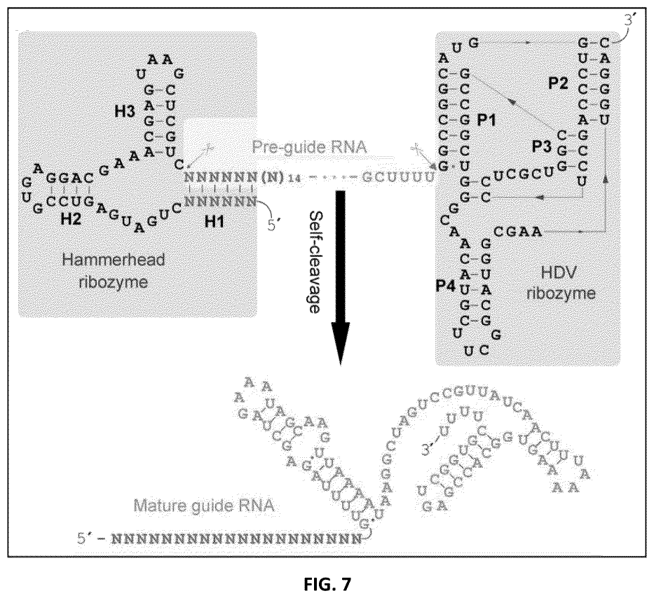

Self-cleaving guide RNAs were also tested. Self-cleaving guide RNAs comprise ribozymes on either side of the guide RNA so that the guide RNA may be driven from a pol II promoter. For example, a hammerhead (HH) ribozyme may be at the 5′ of the guide RNA and a Hepatitis delta virus (HDV) ribozyme may be at the 3′ end of the guide RNA. FIG. 7 provides a schematic of the self-cleaving guide RNA.

In one experiment, a scgRNA comprising a guide RNA flanked by a hammerhead type ribozyme and a hepatitis delta virus type ribozyme was used. The targeting vector utilized for these experiments contains human Factor IX as a biomarker and the scgRNA between arms of homology to the murine albumin locus. The albumin gene was selected as an example of a cell-type specific gene locus. Vectors were designed to contain scgRNA targeting either exon 3 of 4-OH phenylpyruvate dioxygenase (Hpd), exon 4 of Hpd or exon 4 of homogentisic acid dioxygenase (Hgd). Briefly, the vector comprised, in order: left arm of homology (albumin)—p2A (2A peptide)—human Factor IX gene—region of homology (albumin)—HH ribozyme—gRNA—HDV ribozyme—right arm of homology (albumin). The homology arms allow for targeted integration (homologous recombination) and the scgRNA was embedded within an intron. Recombinant adeno-associated virus serotype 8 was generated from these vectors. Neonatal fumarylacetoacetate hydrolase knockout (Fah^{-/-}) mice were given rAAV8 containing the scgRNA Gene Ride vector with 1 of the 3 gRNAs followed by rAAV8 containing SpCas9 driven by the tiny liver specific promoter approximately 4 weeks later. Mice were cycled on 2-(2-nitro-4-trifluoromethylbenzoyl)-1,3-cyclohexanedione (NTBC) to allow selection of hepatocytes that have the correct integration of the targeting vector and hence express gRNAs capable of disrupting selectable genes. In Fah^{-/-} mice, loss of Hpd or Hgd confers a selective growth advantage in hepatocytes. Levels of circulating human Factor IX were assessed at 5, 9, 14 and 18 weeks of age. At 5 and 9 weeks, all mice displayed low levels of human Factor IX. At 14 weeks, mice that had received the scgRNA vector with a gRNA against exon 3 of Hpd began to show increased levels of hF9. These levels significantly increased by 18 weeks of age demonstrating selection for the correctly targeted hepatocytes. Furthermore, mice that received the scgRNA vector with a gRNA targeting exon 4 of Hpd or Hgd showed high levels of hF9 by 18 weeks of age. This finding clearly demonstrates that self-cleaving ribozymes indeed can produce functional gRNAs from a pol 2 promoter and can be used to create selectable gene editing events in the liver in vivo.

The albumin locus was targeted with a GeneRide™ vector incorporating self-cleaving guide RNA against Cypor. In initial experiments, the transgene luciferase was utilized. Briefly, the vector comprised, in order: left arm of homology—p2A (2A peptide)—luciferase gene—region of homology—HH ribozyme—gRNA—HDV ribozyme—right arm of homology. After homologous recombination into the target locus (albumin) the transgene and the self-cleaving guide RNA are expressed.

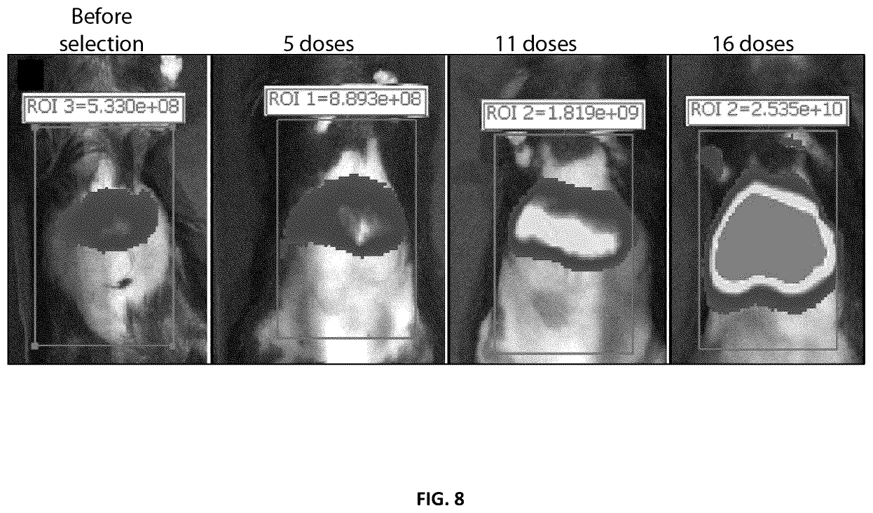

Neonatal mice were injected with this vector and cas9 was given at weaning, followed by APAP injections for 8 weeks. Live luciferase imaging was done on the same mouse at baseline and after 5, 11 and 16 doses. Luciferase expression increased with each dose, reaching a 50× increase (see FIG. 8). At harvest, Cypor staining revealed large negative areas and indel frequency was 21%.

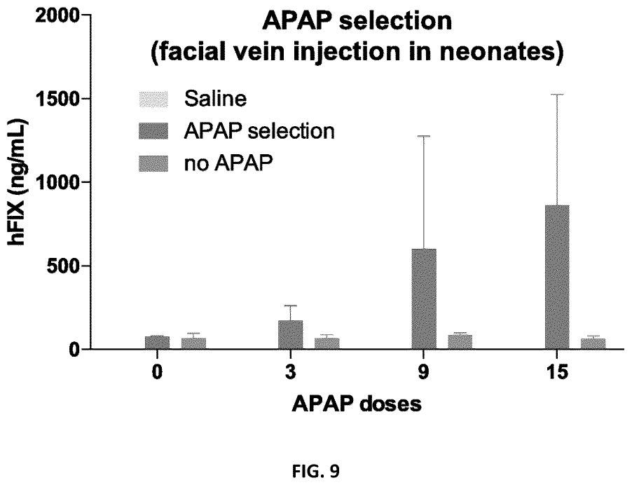

In additional experiments, human Factor IX was expressed as the transgene in the above vector instead of luciferase. The vector was named GRCyporF9. Neonates were injected with AAV GRCyporF9 as neonates and given a single dose of Cas9 at weaning. They were then injected with APAP twice weekly. FIG. 9 shows the human Factor IX levels in blood after 0, 3, 9 and 15 doses of APAP. There was a clear increase in human Factor IX levels in response to the APAP injections. GRCyporF9 injected mice that did not receive APAP, showed no increases and saline controls were also negative.

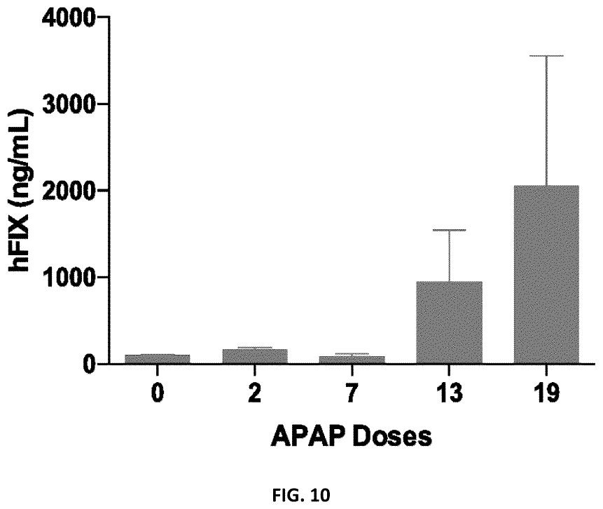

AAV GRCyporF9 was also injected into adult mice along with Cas9. The mice were subsequently given multiple doses of APAP. Human Factor IX levels after 0, 2, 7, 13 and 19 doses are shown in FIG. 10 with clear APAP dependent increases. Mice were killed after 19 doses and the Cypor indel frequency in hepatocytes was measured. All APAP selected mice had indel frequencies of ~20% indicating significant selection.

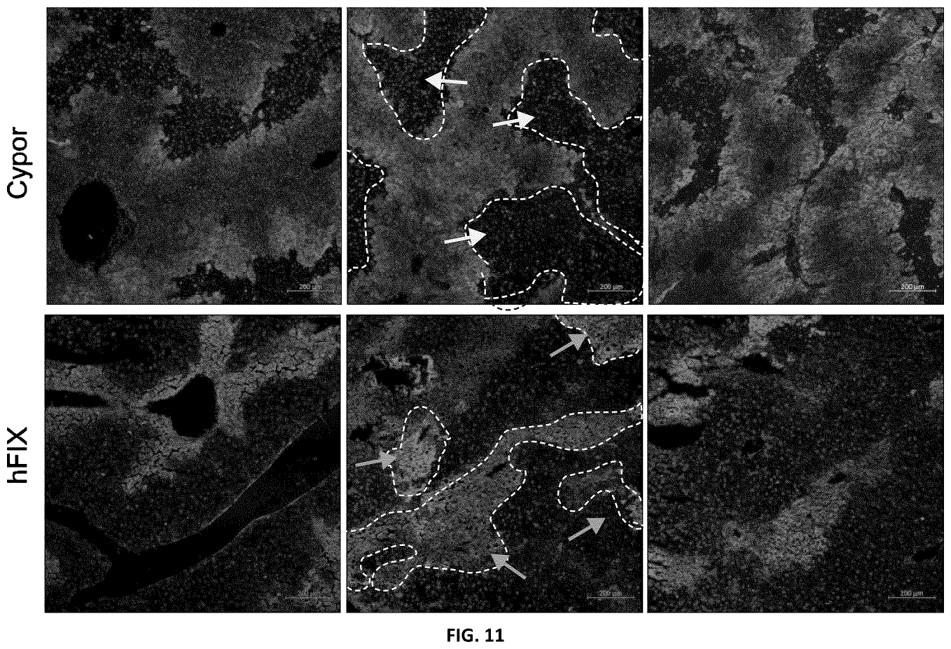

FIG. 11 shows CYPOR (top panels) and human Factor IX (bottom panels) immunohistochemistry in GRCyporF9 treated mice after APAP selection. 3 mice are shown. All APAP treated mice showed extensive areas of Cypor negativity around the central vein (dotted line and arrows in middle panel). The same animals showed large areas of human Factor IX positive mouse hepatocytes in the same distribution pattern (dotted line and arrows in the middle panel). These data show that selected Cypor negative hepatocytes express the therapeutic transgene.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: A method for selectively amplifying and/or expanding a population of cells in a subject, the method comprising the steps of:
a) inhibiting a cytochrome p450 (CYP) enzyme, cytochrome p450 reductase (POR), and/or beta-catenin (CTNNB1) in a cell; and
b) administering a protoxin to a subject having a cell produced by step a),
wherein said protoxin is not converted to a toxic metabolite in cells produced by step a), thereby allowing for amplification and/or expansion of the cells within said subject.

2: The method of claim 1, further comprising introducing a transgene into the cell prior to step b).

3: The method of claim 1, wherein said cell is a hepatocyte.

4: The method of claim 1, wherein step a) is performed in vitro and the cells are administered to the subject before step b).

5: The method of claim 1, wherein step a) is performed in vivo.

6: The method of claim 1, wherein step a) comprises administering an inhibitory nucleic acid molecule or a nucleic acid molecule encoding the inhibitory nucleic acid molecule to the cell or subject, wherein said inhibitory nucleic acid molecule is specific for a CYP enzyme, POR, or CTNNB1.

7: The method of claim 6, wherein said inhibitory nucleic acid molecule is an shRNA.

8: The method of claim 6, wherein step a) comprises administering a vector comprising a nucleic acid molecule encoding the inhibitory nucleic acid molecule and a transgene to said cell or subject.

9: The method of claim 8, wherein said vector is a viral vector, optionally wherein said viral vector is an AAV vector.

10. (canceled)

11: The method of claim 8, wherein said vector is an integrating vector, optionally wherein said integrating vector lacks promoters.

12. (canceled)

13: The method of claim 1, wherein step a) comprises inactivating a CYP enzyme, POR, and/or CTNNB1 by gene editing.

14: The method of claim 13, wherein step a) comprises administering Cas9 and a guide RNA (gRNA) specific for a CYP enzyme, POR, or CTNNB1 gene or a nucleic acid molecule encoding the gRNA to the cell or subject, wherein said gRNA sequence combines with said Cas9 resulting in cleavage of said CYP enzyme, POR, or CTNNB1 gene.

15: The method of claim 14, wherein said gRNA is a self-cleaving guide RNA (scgRNA), wherein said scgRNA comprises a gRNA flanked by a self-cleaving ribozyme at both the 5′ and 3′ ends, optionally wherein said scgRNA is expressed from an RNA polymerase II promoter.

16. (canceled)

17: The method of claim 14, wherein step a) comprises administering a vector comprising a nucleic acid molecule encoding the gRNA and a transgene to said cell or subject.

18: The method of claim 17, wherein said vector is a viral vector, optionally wherein said viral vector is an AAV vector.

19. (canceled)

20: The method of claim 17, wherein said vector is an integrating vector, optionally wherein said integrating vector lacks promoters.

21. (canceled)

22: The method of claim 1, wherein said protoxin is administered in an amount sufficient to result in mild hepatotoxicity in the subject, or
wherein said protoxin is administered in an amount sufficient to result in moderate hepatotoxicity in the subject.

23. (canceled)

24: The method of claim 1, wherein said protoxin is administered in an amount sufficient to elevate in the subject's levels of at least one liver enzyme selected from the group comprising alanine aminotransferase, aspartate transaminase, alkaline phosphatase, and gamma-glutamyl transpeptidase.

25: The method of claim 1, wherein said protoxin is selected from the group consisting of acetaminophen, retrorsine, cyclophosphamide, tamoxifen, ketoconazole, tramadol, tacrine, lasiocarpine, senkirkin, dacarbazine, methoxymorpholinyl doxorubicin (PNU 152243), ifosfamide, trofosfamide, pradefovir, MB07133, buparvaquone hydroxylamine, nabumetone hydroxyimine, DB289, furamidine, sibrafibin, ximelagatran, guanoxabenz, AQ4N, 4-ipomeanol, clopidogrel, V-PYRRO/NO, V-PROLI/NO, and tegafur.

26: The method of claim 1, wherein the protoxin is acetaminophen, optionally
wherein the acetaminophen is administered to the subject at a dose of 6 grams per day on consecutive days, or
wherein the acetaminophen is administered to the subject at a single dose of 10 grams.

27. (canceled)

28. (canceled)
